(19) United States
(12) Patent Application Publication
(10) Pub. No.: US 2025/0339134 A1
(43) Pub. Date: Nov. 6, 2025

(54) IMPLANT DELIVERY SYSTEMS

(71) Applicant: Teleflex Life Sciences LLC, Wilmington, DE (US)

(72) Inventors: Curtis Yarra, Oakland, CA (US); Alexander Gordon, San Carlos, CA (US); Ernest Cabreza, Dublin, CA (US); Erik Noel, San Ramon, CA (US); Brian Wilkins, Pleasanton, CA (US); Manoj Kumar Singh, Pleasanton, CA (US); David Carpenter, Jaffrey, NH (US)

(73) Assignee: Teleflex Life Sciences LLC, Wilmington, DE (US)

(21) Appl. No.: 19/197,707

(22) Filed: May 2, 2025

Related U.S. Application Data
(60) Provisional application No. 63/641,534, filed on May 2, 2024.
(60) Provisional application No. 63/641,570, filed on May 2, 2024.
(60) Provisional application No. 63/641,720, filed on May 2, 2024.
(60) Provisional application No. 63/641,550, filed on May 2, 2024.
(60) Provisional application No. 63/641,745, filed on May 2, 2024.
(60) Provisional application No. 63/641,778, filed on May 2, 2024.

(51) Int. Cl. A61B 17/00; A61B 17/04; A61B 17/34

(52) U.S. Cl. CPC A61B 17/00234; A61B 17/0401; A61B 17/34; A61B 2017/00274; A61B 2017/00353; A61B 2017/00407; A61B 2017/0409; A61B 2017/0464

(57) ABSTRACT

Implant delivery systems are configured to deploy multiple implants into targeted tissue within a patient without disengaging a delivery component of the systems from its insertion site in the patient. Various combinations of spooling assemblies, spring-loaded features, manifolds, indexing mechanisms, gearings, and manually engageable actuators operate to deploy the implants in a controlled, serial fashion. Implants or portions thereof are pre-loaded into the handle assemblies of the delivery systems or contained in removable cartridges configured for coupling to the handle assemblies.

### CROSS REFERENCE TO RELATED APPLICATIONS

This application claims priority to U.S. Provisional Patent Application No. 63/641,534, filed on May 2, 2024, titled “Implant Delivery System” and U.S. Provisional Patent Application No. 63/641,778, filed on May 2, 2024, titled “Implant Delivery System” and U.S. Provisional Patent Application No. 63/641,570, filed on May 2, 2024, titled “Implant Delivery System” and U.S. Provisional Patent Applicant No. 63/641,720, filed on May 2, 2024, titled “Implant Delivery System” and U.S. Provisional Patent Application No. 63/641,745, filed on May 2, 2024, titled “Implant Delivery System” and U.S. Provisional Patent Application No. 63/641,550, filed on May 2, 2024, titled “Implant Delivery System.” The contents of these applications are hereby incorporated by reference in their entireties herein.

### TECHNICAL FIELD

The subject matter of this patent document relates to the field of medical devices. More particularly, but not by way of limitation, the subject matter relates to medical devices, systems, and methods for relieving pressure on a prostatic urethra by compressing at least a portion of a prostate gland.

### BACKGROUND

Benign Prostatic Hyperplasia (“BPH”) is one of the most common medical conditions that affect men, especially elderly men. It has been reported that, in the United States, more than half of all men have histopathologic evidence of BPH by age 60 and, by age 85, approximately 9 out of 10 men suffer from the condition. Moreover, the incidence and prevalence of BPH are expected to increase as the average age of the population in developed countries increases.

The prostate gland enlarges throughout a man's life. In some men, the prostatic capsule around the prostate gland may prevent the prostate gland from enlarging further. This causes the inner end of the prostate gland to squeeze the urethra. This pressure on the urethra increases resistance to urine flow through the end of the urethra enclosed by the prostate. Thus, the urinary bladder has to exert more pressure to force urine through the increased resistance of the urethra. Chronic over-exertion causes the muscular walls of the urinary bladder to remodel and become stiffer. This combination of increased urethral resistance to urine flow and stiffness and hypertrophy of urinary bladder walls leads to a variety of lower urinary tract symptoms (LUTS) that may severely reduce the patient's quality of life. These symptoms include weak or intermittent urine flow while urinating, straining when urinating, hesitation before urine flow starts, feeling that the bladder has not emptied completely even after urination, dribbling at the end of urination or leakage afterward, increased frequency of urination particularly at night, urgent need to urinate etc.

In addition to patients with BPH, LUTS may also be present in patients with prostate cancer, prostate infections, and chronic use of certain medications (e.g. ephedrine, pseudoephedrine, phenylpropanolamine, antihistamines such as diphenhydramine, chlorpheniramine, etc.) that cause urinary retention especially in men with prostate enlargement.

Despite extensive efforts in both the medical device and pharmacotherapeutic fields, current treatments for BPH remain only partially effective and are burdened with significant side effects. Thus, there remains a need for the development of new devices, systems and methods for treating BPH as well as other conditions in which one tissue or anatomical structure impinges upon or compresses another tissue or anatomical structure.

### SUMMARY

Disclosed herein are devices, systems, and methods for compressing at least a portion of a prostate gland, thereby alleviating pressure on the prostatic urethra, by deploying one or more anchor assemblies or implants into the targeted prostatic tissue. Successful deployment of the implant(s) may effectively treat BPH, among other conditions, for example those in which retraction or compression of enlarged or inflamed tissue is desired.

In embodiments, the prostatic implants are configured to anchor simultaneously to the outer prostatic capsule, and also a urethral side, of the lobe of an enlarged prostate, such as a median or lateral lobe. Each implant may include a distal anchor portion (or capsular tab, “CT”) configured to anchor on the outside of the prostatic capsule. An elongate middle portion, such as a suture, may connect the distal anchor portion to a proximal anchor portion (or urethral endpiece, “UE”) configured to anchor to a urethral side of the lobe. Once the distal anchor portion is implanted, the elongate middle portion may be tensioned and the proximal anchor portion subsequently attached thereto. Attachment of the proximal anchor portion may lock the tensioned middle portion in place, compressing the prostatic tissue between the distal and proximal anchors and relieving constriction of the prostatic urethra.

Using preexisting delivery systems, each implant or component thereof is typically provided in a separate delivery device or cartridge configured to couple therewith. Where a cartridge containing an implant is used, the cartridge may be loaded into a delivery device, which is then activated to deploy the implant into the targeted tissue by transferring mechanical energy to the cartridge or internal subassembly. The delivery device, or at least its elongate, tubular shaft assembly, may then be then removed from the patient, and the spent cartridge replaced with a new cartridge containing a second implant. The delivery device (or more specifically, the shaft assembly) may then be re-inserted and the deployment process repeated. Multiple implants are often necessary to complete a single procedure, thus necessitating multiple cartridge exchanges and the associated removals/reinsertions of the shaft assembly.

Disclosed herein are delivery devices, assemblies, and components thereof configured to deploy multiple prostatic implants to targeted prostatic tissue without removing or disengaging the device(s) or component(s) thereof, e.g., the elongate shaft assembly, from the patient during the procedure. Embodiments of a delivery device are configured to be reloaded “on the fly” with one or more cartridges, each cartridge carrying one or more prostatic implants or components thereof, e.g., the distal anchor and suture, during a procedure. Also disclosed are embodiments of “multi-fire” delivery devices configured to hold multiple prostatic implants simultaneously for serial insertion into prostatic tissue during a procedure. Mechanisms for assembling each implant, for example by attaching a urethral endpiece to each of the serially deployed anchor assemblies, are also disclosed, as are systems configured to prevent device jamming in the event of a CT/suture pull-through.

Embodiments of the delivery devices include various subassemblies mobilized via one or more actuators or manually accessible structures, the operation of which is coordinated and synchronized to ensure accurate and precise implantation of each implant. One or more reloadable cartridge and/or multi-fire embodiments may include various actuators, spooling assemblies, spring-loaded features, indexing mechanisms, gearings, etc., which may be interchangeable or modifiable across certain embodiments. Embodiments may also include structure configured to receive a conventional remote viewing device (e.g., an endoscope) so that the steps being performed at the interventional site can be observed.

In some embodiments, a delivery device configured to deploy multiple implant assemblies to a prostate gland of a patient without removing an elongate delivery shaft of the delivery device from the patient includes an elongate delivery shaft attached to a handle assembly. The handle assembly may include a first spool member configured to couple with multiple implant assemblies simultaneously, each implant assembly comprising a distal anchor component and a suture attached thereto (“CT/suture”), the distal anchor component configured to anchor to a prostatic capsule of the prostate gland, and the suture configured to be placed within the prostate gland. The handle assembly may further include a second spool member configured to couple with a delivery needle configured to pierce the prostate gland. The handle assembly may further include one or more actuators configured to cause deployment of the implant assemblies in serial fashion.

In some embodiments, the one or more actuators are manually engageable. In some embodiments, at least one of the one or more actuators comprises a gearing portion inside the handle assembly. In some embodiments, at least one of the one or more actuators may be directly or operatively coupled to the first spool member, the second spool member, or both.

In some embodiments, the delivery device may further include multiple urethral endpieces attachable to the implant assemblies, where the one or more actuators are further configured to cause attachment of each of the implant assemblies to one of the urethral endpieces. In some embodiments, the one or more actuators includes a first actuator and a second actuator. In some embodiments, actuation of the first actuator drives deployment of each implant assembly. In some embodiments, actuation of the second actuator may index the urethral endpieces in the elongate delivery shaft. In some embodiments, the urethral endpieces may be aligned end-to-end within the elongate delivery shaft. In some embodiments, the first actuator comprises a trigger. In some embodiments, the second actuator comprises a lever member.

In some embodiments, the first spool member and the second spool member rotate at different speeds in response to actuation of at least one of the one or more actuators. In some embodiments, the second spool member is further configured to unwind and wind the delivery needle in response to actuation of at least one of the one or more actuators. In some embodiments, the one or more actuators are configured to cause deployment of the implant assemblies in serial fashion via a ratcheting mechanism. In some embodiments, the ratcheting mechanism comprises an assembly comprising a ratchet plate defining a suture deployment track configured to receive and accommodate stepwise movement of a movable component. In some embodiments, the first spool member may be configured to move relative to the second spool member. In some embodiments, the first spool member may define a plurality of circumferential tracks, each circumferential track configured to accommodate one implant assembly. In some embodiments, movement of the first spool member relative to the second spool member may cause the implant assemblies to unwind from the first spool member and feed into the delivery needle via an opening defined by the second spool member.

In some embodiments, a method for compressing prostate tissue involves advancing an elongate shaft assembly of a delivery device through a urethra until a distal end of the elongate shaft assembly is positioned adjacent to the prostate tissue, where the delivery device further includes a handle assembly attached to a proximal end of the elongate shaft assembly. The handle assembly may include a spooling assembly featuring a first spool member and a second spool member, the first spool member configured to couple with multiple prostatic implant assemblies simultaneously, and the second spool member configured to couple with a hollow delivery needle configured to receive the prostatic implant assemblies and pierce the prostate tissue. The handle assembly may also include one or more actuators configured to cause deployment of the prostatic implant assemblies into the prostate tissue in serial fashion. The method may also involve deploying the prostatic implant assemblies into the prostate tissue without removing the elongate shaft assembly from the urethra. In some embodiments, deploying the prostatic implant assemblies into the prostate tissue comprises actuating a manually engageable actuator two or more times.

These and other examples and objects of the present devices and related methods will be set forth in the following Detailed Description. This Overview is intended to provide non-limiting examples of the present subject matter. The Detailed Description below is included to provide further information about the present devices and related methods. Neither is intended to provide an exclusive or exhaustive explanation of the present devices and methods because this disclosure is written for those of ordinary skill in the art.

### BRIEF DESCRIPTION OF THE DRAWINGS

In the drawings, like numerals can be used to describe similar features and components throughout the several views. The drawings illustrate generally, by way of example but not by way of limitation, various embodiments discussed in the present patent document.

FIG. 1A illustrates a cross-sectional view of the anatomy surrounding a prostate in a human subject.

FIG. 1B illustrates an enlarged cross-sectional view of the anatomy surrounding a prostate.

FIG. 2 shows a coronal section through the lower abdomen of a male human suffering from BPH showing a hypertrophied prostate gland treated with an embodiment of the device of the present disclosure.

FIGS. 3A-3G show the various steps of a method of treating a prostate gland using the implant(s) and devices shown and described herein.

FIG. 4A shows a perspective view of an example of an implant in accordance with embodiments disclosed herein, for example as shown in FIGS. 2-3G.

FIG. 4B shows a side view of an example of an implant delivery device configured to deliver the implant shown in FIG. 4A.

FIG. 5A shows a partially transparent right-side view of a portion of an implant delivery device in accordance with embodiments disclosed herein.

FIG. 5B shows a partially transparent left-side view of the portion of the implant delivery device shown in FIG. 5A in accordance with embodiments disclosed herein.

FIG. 6A shows a removable cartridge compatible with the implant delivery device shown in FIGS. 5A and 5B in accordance with embodiments disclosed herein.

FIG. 6B shows another view of a portion of the removable cartridge shown in FIG. 6A in accordance with embodiments disclosed herein.

FIG. 6C shows another view of a portion of the removable cartridge shown in FIG. 6A in accordance with embodiments disclosed herein.

FIG. 6D shows another view of a portion of the removable cartridge shown in FIG. 6A in accordance with embodiments disclosed herein.

FIG. 7A shows another partially transparent view of a portion of the delivery device and removable cartridge shown in FIGS. 5A-6D in accordance with embodiments disclosed herein.

FIG. 7B shows another partially transparent view of a portion of the delivery device and removable cartridge shown in FIGS. 5A-6D in accordance with embodiments disclosed herein.

FIG. 7C shows another view of a portion of the delivery device and removable cartridge shown in FIGS. 5A-6D in accordance with embodiments disclosed herein.

FIG. 7D shows another view of a portion of the delivery device and removable cartridge shown in FIGS. 5A-6D in accordance with embodiments disclosed herein.

FIG. 7E shows another view of a portion of the delivery device and removable cartridge shown in FIGS. 5A-6D in accordance with embodiments disclosed herein

FIG. 7F shows another view of a portion of the delivery device and removable cartridge shown in FIGS. 5A-6D in accordance with embodiments disclosed herein

FIG. 8A shows a partially transparent right-side view of a portion of an implant delivery device in accordance with embodiments disclosed herein.

FIG. 8B shows another view of a portion of the implant delivery device shown in FIG. 8A in accordance with embodiments disclosed herein.

FIG. 9 shows an exploded view of components of the implant delivery device shown in FIGS. 8A and 8B in accordance with embodiments disclosed herein.

FIG. 10 shows an exploded view of components of the implant delivery device shown in FIGS. 8A-9 in accordance with embodiments disclosed herein.

FIG. 11 shows a perspective view of a component of the implant delivery device shown in FIGS. 8A-10 in accordance with embodiments disclosed herein.

FIG. 12 shows a view of a component of the implant delivery device shown in FIGS. 8A-11 in accordance with embodiments disclosed herein.

FIG. 13 shows a view of a component of the implant delivery device shown in FIGS. 8A-12 in accordance with embodiments disclosed herein.

FIG. 14A shows a partially transparent side view of the implant delivery device shown in FIGS. 8A-13 during an implant deployment process implemented in accordance with embodiments disclosed herein.

FIG. 14B shows another partially transparent side view of the implant delivery device shown in FIGS. 8A-14A during an implant deployment process implemented in accordance with embodiments disclosed herein.

FIG. 14C shows another partially transparent side view of the implant delivery device shown in FIGS. 8A-14B during an implant deployment process implemented in accordance with embodiments disclosed herein.

FIG. 14D shows another partially transparent side view of the implant delivery device shown in FIGS. 8A-14C during an implant deployment process implemented in accordance with embodiments disclosed herein.

FIG. 15A shows an exploded view of components of an implant delivery device in accordance with embodiments disclosed herein.

FIG. 15B shows a partially transparent view of components of the implant delivery device shown in FIG. 15A in accordance with embodiments disclosed herein.

FIG. 15C shows a perspective view of components of the implant delivery device shown in FIG. 15A in accordance with embodiments disclosed herein.

FIG. 15D shows a plan view of components of the implant delivery device shown in FIG. 15A in accordance with embodiments disclosed herein.

FIG. 15E shows a perspective view of components of the implant delivery device shown in FIG. 15A in accordance with embodiments disclosed herein.

FIG. 15F shows a perspective view of two components of the implant delivery device shown in FIG. 15A in accordance with embodiments disclosed herein.

FIG. 16 shows an exploded view of components of an implant delivery device in accordance with embodiments disclosed herein.

FIG. 17A shows a partially transparent side view of a portion of the implant delivery device shown in FIG. 16 in accordance with embodiments disclosed herein.

FIG. 17B shows a close-up side view of a portion of the implant delivery device shown in FIG. 16 in accordance with embodiments disclosed herein.

FIG. 18 shows an exploded view of components of an implant delivery device in accordance with embodiments disclosed herein.

FIG. 19 shows a perspective view of one of the components shown in FIG. 18 in accordance with embodiments disclosed herein.

FIG. 20 shows a perspective view of another one of the components shown in FIG. 18 in accordance with embodiments disclosed herein.

FIG. 21A shows a perspective view of another one of the components shown in FIG. 18 in accordance with embodiments disclosed herein.

FIG. 21B shows another perspective view of the component shown in FIG. 21A in accordance with embodiments disclosed herein.

FIG. 22 shows a view of another one of the components shown in FIG. 18 in accordance with embodiments disclosed herein.

FIG. 23 shows a side view of a portion of an implant delivery device including the components shown in FIGS. 18-22 in accordance with embodiments disclosed herein.

FIG. 24A shows a side view of another component of the implant delivery device shown in FIG. 23 in accordance with embodiments disclosed herein.

FIG. 24B shows a perspective view of another component of the implant delivery device shown in FIG. 23 in accordance with embodiments disclosed herein.

FIG. 25 shows a view of another component of the implant delivery device shown in FIG. 23 in accordance with embodiments disclosed herein.

FIG. 26A shows perspective views of components of an implant delivery device in accordance with embodiments disclosed herein.

FIG. 26B shows a side view of a component shown in FIG. 26A in accordance with embodiments disclosed herein.

FIG. 27A shows a snapshot of the operation of the components shown in FIG. 26A during an implant deployment process implemented in accordance with embodiments disclosed herein.

FIG. 27B shows another snapshot of the operation of the components shown in FIG. 26A during an implant deployment process implemented in accordance with embodiments disclosed herein.

FIG. 27C shows another snapshot of the operation of the components shown in FIG. 26A during an implant deployment process implemented in accordance with embodiments disclosed herein.

FIG. 27D shows a close-up view of components shown in FIG. 26A in accordance with embodiments disclosed herein.

FIG. 28 shows a view of a retainer or implant in accordance with embodiments disclosed herein.

FIG. 29A shows a view of a portion of an implant delivery device configured to deploy the implant shown in FIG. 28 in accordance with embodiments disclosed herein.

FIG. 29B shows a view of the portion of a delivery device shown in FIG. 29A during the deployment of the implant shown in FIG. 28 in accordance with embodiments disclosed herein.

FIG. 30 shows a perspective view of a portion of an implant delivery device in accordance with embodiments disclosed herein.

FIG. 31 shows a side view of a portion of an implant delivery device in accordance with embodiments disclosed herein.

FIG. 32A shows a view of components of an implant delivery device in accordance with embodiments disclosed herein.

FIG. 32B shows a view of components of the implant delivery device shown in FIG. 32A in accordance with embodiments disclosed herein.

FIG. 33 shows a view of a portion of the implant delivery device shown in FIG. 32B in accordance with embodiments disclosed herein.

FIG. 34A shows a partially transparent side view of the implant delivery device shown in FIG. 33 during an implant deployment process implemented in accordance with embodiments disclosed herein.

FIG. 34B shows another partially transparent side view of the implant delivery device shown in FIG. 33 during an implant deployment process implemented in accordance with embodiments disclosed herein.

FIG. 34C shows another partially transparent side view of the implant delivery device shown in FIG. 33 during an implant deployment process implemented in accordance with embodiments disclosed herein.

FIG. 34D shows another partially transparent side view of the implant delivery device shown in FIG. 33 during an implant deployment process implemented in accordance with embodiments disclosed herein.

FIG. 34E shows another partially transparent side view of the implant delivery device shown in FIG. 33 during an implant deployment process implemented in accordance with embodiments disclosed herein.

FIG. 34F shows another partially transparent side view of the implant delivery device shown in FIG. 33 during an implant deployment process implemented in accordance with embodiments disclosed herein.

FIG. 34G shows another partially transparent side view of the implant delivery device shown in FIG. 33 during an implant deployment process implemented in accordance with embodiments disclosed herein.

FIG. 34H shows another partially transparent side view of the implant delivery device shown in FIG. 33 during an implant deployment process implemented in accordance with embodiments disclosed herein.

FIG. 35 shows a perspective view and an exploded view of components of an implant delivery device in accordance with embodiments disclosed herein.

FIG. 36 shows a perspective view of components shown in FIG. 35 in accordance with embodiments disclosed herein.

FIG. 37 shows a perspective view of components shown in FIG. 35 in accordance with embodiments disclosed herein.

FIG. 38 shows a perspective view of a distal portion of an implant delivery device in accordance with embodiments disclosed herein.

FIG. 39 shows an exploded view of components of the implant delivery device shown in FIG. 38 in accordance with embodiments disclosed herein.

FIG. 40 shows a partially transparent view of components of the implant delivery device shown in FIG. 38 in accordance with embodiments disclosed herein.

FIG. 41 shows a partially transparent view of components of the implant delivery device shown in FIG. 38 in accordance with additional embodiments disclosed herein.

FIG. 42A shows a side view of the implant delivery device shown in FIG. 38 during an implant deployment process implemented in accordance with embodiments disclosed herein.

FIG. 42B shows another side view of the implant delivery device shown in FIG. 38 during an implant deployment process implemented in accordance with embodiments disclosed herein.

FIG. 42C shows another side view of the implant delivery device shown in FIG. 38 during an implant deployment process implemented in accordance with embodiments disclosed herein.

FIG. 42D shows another side view of the implant delivery device shown in FIG. 38 during an implant deployment process implemented in accordance with embodiments disclosed herein.

FIG. 43A shows a perspective view of a distal portion of a delivery device during an implant deployment process implemented in accordance with embodiments disclosed herein.

FIG. 43B shows another perspective view of a distal portion of the delivery device shown in FIG. 43A during an implant deployment process implemented in accordance with embodiments disclosed herein.

FIG. 43C shows another perspective view of a distal portion of the delivery device shown in FIG. 43A during an implant deployment process implemented in accordance with embodiments disclosed herein.

FIG. 43D shows another perspective view of a distal portion of the delivery device shown in FIG. 43A during an implant deployment process in accordance with embodiments disclosed herein.

FIG. 44A shows a perspective view of an implant delivery device during an implant deployment process implemented in accordance with embodiments disclosed herein.

FIG. 44B shows another perspective view of the delivery device shown in FIG. 44A during an implant deployment process implemented in accordance with embodiments disclosed herein.

FIG. 44C shows another perspective view of the delivery device shown in FIG. 44A during an implant deployment process implemented in accordance with embodiments disclosed herein.

FIG. 44D shows another perspective view of the delivery device shown in FIG. 44A during an implant deployment process implemented in accordance with embodiments disclosed herein.

FIG. 44E shows another perspective view of various components of the delivery device shown in FIG. 44A during an implant deployment process implemented in accordance with embodiments disclosed herein.

FIG. 44F shows another perspective view of various components of the delivery device shown in FIG. 44A during an implant deployment process implemented in accordance with embodiments disclosed herein.

FIG. 44G shows another perspective view of various components of the delivery device shown in FIG. 44A during an implant deployment process implemented in accordance with embodiments disclosed herein.

FIG. 44H shows another perspective view of various components of the delivery device shown in FIG. 44A during an implant deployment process implemented in accordance with embodiments disclosed herein.

FIG. 44I shows another perspective view of various components of the delivery device shown in FIG. 44A during an implant deployment process implemented in accordance with embodiments disclosed herein.

FIG. 45 shows an exploded view of components of an implant delivery device in accordance with embodiments disclosed herein.

FIG. 46 shows a cross-sectional side view of the assembled components shown in FIG. 45 in accordance with embodiments disclosed herein.

FIG. 47A shows perspective views of the delivery device components shown in FIGS. 45-46 during an implant deployment process implemented in accordance with embodiments disclosed herein.

FIG. 47B shows additional perspective views of the delivery device components shown in FIGS. 45-46 during an implant deployment process implemented in accordance with embodiments disclosed herein.

FIG. 47C shows additional perspective views of the delivery device components shown in FIGS. 45-46 during an implant deployment process implemented in accordance with embodiments disclosed herein.

FIG. 48A shows an exploded view and partially transparent side view of components of an implant delivery device in accordance with embodiments disclosed herein.

FIG. 48B shows a side view of assembled components of the implant delivery device shown in FIG. 48A in accordance with embodiments disclosed herein.

FIG. 48C shows a spool member included in the implant delivery device shown in FIG. 48A.

FIG. 48D shows an exploded view of a subset of components of the implant delivery device shown in FIG. 48A.

FIG. 48E shows a partially exploded and assembled view of the components shown in FIG. 48D.

FIG. 48F shows plan, side, and assembled views of components shown in FIG. 48D.

FIG. 48G shows plan and side views of a ratchet plate included in the implant delivery device shown in FIG. 48A.

FIG. 48H shows plan and side views of a return plate included in the implant delivery device shown in FIG. 48A.

FIG. 48I shows a close-up view of a drive gear member and surrounding components within the implant delivery device shown in FIG. 48A.

FIG. 48J shows a close-up view of components of the implant delivery device shown in FIG. 48A.

FIG. 48K shows a partially transparent perspective view of the handle assembly of the implant device shown in FIG. 48A.

FIG. 48L shows a front view of the components shown in FIG. 48K.

FIG. 48M shows a partially transparent side view of two spool members included in the assembly shown in FIG. 48K.

FIG. 48N shows a perspective view of components of the handle assembly shown in FIG. 48A.

FIG. 48O shows front and side views of the components shown in FIG. 48N in a first configuration.

FIG. 48P shows a perspective view of the components shown in FIG. 48O in a second configuration.

FIG. 48Q shows a partially transparent left-side view of a CT ratcheting assembly included in the implant delivery system shown in FIG. 48A.

FIG. 48R shows a partially transparent right-side view of the CT ratcheting assembly shown in FIG. 48Q.

FIG. 49 shows a schematic representation of an automated implant delivery system and associated features in accordance with embodiments disclosed herein.

The drawing figures are not necessarily to scale. Certain features and components may be shown exaggerated in scale or in schematic form and some details may not be shown in the interest of clarity and conciseness.

### DETAILED DESCRIPTION

The present devices and associated methods provide clinicians with means to treat an enlarged prostate, which may be a symptom of BPH, to alleviate its impingement on the adjacent prostatic urethra. Implants disclosed herein can be placed using a method for compressing a prostate gland or portion thereof according to the following description.

As used herein, the terms “prostatic implant” and “implant” and “anchor assembly” and “retainer” may be used interchangeably. Each implant, once fully assembled, may include a distal anchor or capsular tab (“CT”), a connector member, portion or suture (“suture”), and a proximal anchor or urethral endpiece (“UE”). The CT and suture may be provided together as a unitary component or assembly, with the CT attached, fixed, integrally formed with, or coupled to one end of the suture. Accordingly, the CT and suture may be referred to as a “CT/suture” or “CT/suture assembly” herein. The UE may be provided as a separate component that is attached to the CT/suture after deploying the CT/suture to the targeted tissue, for instance after the CT has emerged from the distal end of the delivery needle, at or beyond the outer surface of the prostatic capsule, and the suture has been implanted within the prostatic tissue. Embodiments include attaching the UE to the suture of a CT/suture assembly after tightening the CT against the outer capsular surface and tensioning the suture through the targeted tissue. In various examples, the terms “capsular tab” and “CT” and “distal anchor” may be used interchangeably. In various examples, the terms “urethral endpiece” and “UE” and “proximal anchor” may be used interchangeably.

The terms “actuator” and “actuator member” may be used interchangeably in some embodiments disclosed herein. In some examples, an “actuator” or “actuator member” may include or be synonymous with a manually engageable portion, member, part, or component (such as a trigger, button, lever, dial, switch, toggle, or knob) configured to be actuated, for example via manually induced movement (such as a trigger pull, button press, or lever sweep). Accordingly, an “actuator” may include or be used interchangeably with a “trigger” or “trigger assembly” in some embodiments.

As used herein, the term “suture” may be used to represent a connector, connector member, connector portion, or elongate middle portion or member of an implant extending between a distal anchor and a urethral endpiece, through the prostatic tissue.

The term “member” may be used herein to represent a subcomponent or subassembly of a larger component or assembly, or the term may represent the larger component or assembly itself. For instance, the terms “spool” and “spool member” may be used interchangeably. The terms “element” and “member” and “component” may also be used interchangeably herein.

The terms “elongate member” and “shaft” and “shaft assembly” may refer to the same or similar components and may be used interchangeably herein. Embodiments of a shaft assembly may include components configured to access a treatment site within a prostatic urethra, deploy one or more implants, and/or visualize the procedure from within the patient. Examples of the shaft assembly may include a scope tube configured to accommodate an endoscopic instrument within a lumen of the scope tube. Examples of a shaft assembly may include one or more components of a needle assembly, a suture assembly, and/or a cutter assembly, non-limiting examples of which are disclosed in U.S. Pat. No. 11,298,115 and U.S. Patent Application Publication No. 2021/0378658, the entire contents of each of which are incorporated by reference herein.

The term “sheath” may be used interchangeably with “sheath device” and/or “introducer sheath.” Embodiments of the sheaths disclosed herein can be configured to receive an elongate portion, e.g., shaft assembly, of the delivery devices disclosed herein.

The term “procedure” may refer to a medical treatment method used to compress at least a portion of an anatomical structure or tissue, including tissue of the prostate gland, which may be enlarged relative to a corresponding healthy tissue.

The term “user” may refer to a clinician, doctor, nurse, or medical professional performing a procedure described herein, which may involve the implantation of one or more implants within a targeted portion of prostatic tissue, which may be enlarged relative to normal prostatic tissue. In some examples, the term “user” may refer to more than one person, including two or more medical professionals working together to perform a procedure.

The terms “deploy” and “deliver” and “implant” may be used interchangeably herein, referring to the release and/or ejection of a disclosed implant or subassembly from a delivery device into a tissue being targeted. Accordingly, for instance, an implant may be fully deployed, delivered, or implanted when the distal anchor of an implant is positioned at an outer surface of the prostatic capsule, the suture has been advanced through and tensioned within the targeted prostate lobe, and the UE has been attached to the tensioned suture at the urethral side of the lobe.

The term “fire” may be used herein to describe the deployment, insertion into tissue, and/or distal advancement of one or more components of the delivery devices disclosed herein, such as a needle defining an elongate inner lumen containing at least a portion of a CT/suture assembly, and/or the CT/suture assembly itself. A delivery device configured to deploy multiple implants during a given procedure may thus be referred to as a “multi-fire” device.

The terms “serial” and “successive” may both refer to the one-by-one deployment of multiple implants and/or assemblies thereof, for example during a single procedure, and may thus be used interchangeably herein.

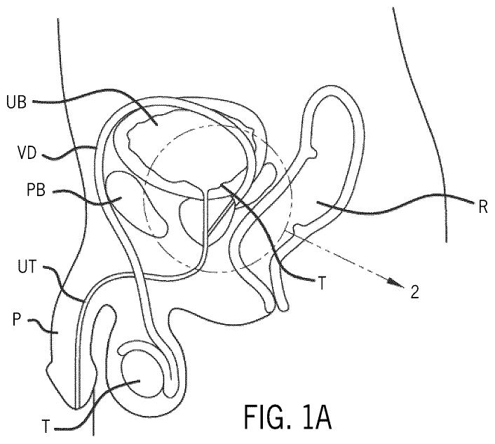

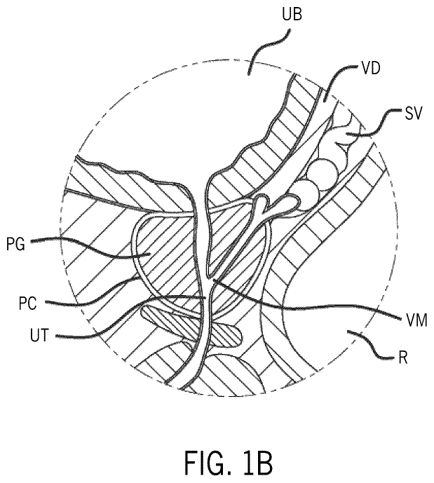

FIGS. 1A and 1B illustrate various features of the urological anatomy of a human subject. The prostate gland PG is a walnut-sized muscular gland located adjacent the urinary bladder UB. The urethra UT runs through the prostate gland PG. The prostate gland PG secretes fluid that protects and nourishes sperm. The prostate also contracts during sperm ejaculation to expel semen and provide a valve to keep urine out of the semen. A firm prostatic capsule PC surrounds the prostate gland PG.

The urinary bladder UB holds urine. The vas deferentia VD define ducts through which semen is carried, and the seminal vesicles SV secrete seminal fluid. The rectum R is the end segment of the large intestine through which waste is dispelled. The urethra UT carries both urine and semen out of the body. Thus, the urethra is connected to the urinary bladder UB and provides a passageway to the vas deferentia VD and seminal vesicles SV.

The trigone T is a smooth triangular end of the bladder. It is sensitive to expansion and signals the brain when the urinary bladder UB is full. The verumontanum VM is a crest in the wall of the urethra UT where the seminal ducts enter. The prostatic urethra is the section of the urethra UT that extends through the prostate.

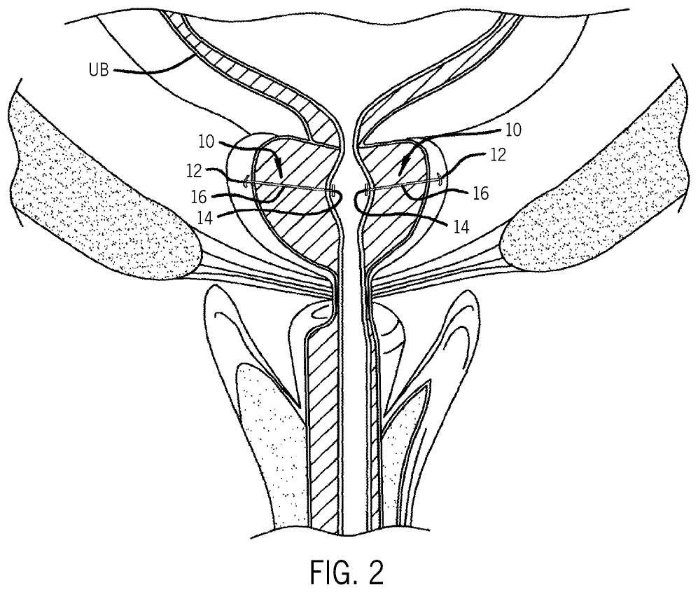

FIG. 2 shows a coronal section through the lower abdomen of a male human suffering from BPH, showing a hypertrophied prostate gland treated with embodiments of the devices of the present invention. It has been discovered that the enlarged prostate gland is compressible and can be retracted so as to relieve the pressure from the urethra. In accordance with one embodiment of the present invention, a retaining device can be placed through the prostate gland in order to relieve the pressure on the urethra. In FIG. 2, a retainer or implant 10 is implanted in the prostate gland. Implant 10 comprises a distal anchor 12 and a proximal anchor 14. After assembly of the implant 10, the distal anchor 12 and proximal anchor 14 are connected by a middle portion or connector 16, which may comprise, include, or resemble a suture. The radial distance from the urethra to distal anchor 12 is greater than the radial distance from the urethra to the proximal anchor 14. The distance or tension between the anchors is sufficient to compress, displace or change the orientation of an anatomical region between distal anchor 12 and proximal anchor 14. The connector 16 may be substantially inelastic so as to maintain a constant force or distance between the proximal and distal anchors, or it may be elastic to facilitate drawing the proximal and distal anchors closer together. In the embodiment shown in FIG. 2, distal anchor 12 is located on the outer surface of the capsule of prostate gland CP and acts as a capsular anchor. Alternatively, distal anchor 12 may be embedded inside the tissue of prostate gland PG, or in the surrounding structures around the prostate, such as periosteum of the pelvic bones, within the bones themselves, pelvic fascia, muscles traversing the pelvis or bladder wall. Also, in the embodiment shown in FIG. 2, proximal anchor 14 may be located on the inner wall of the urethra UT, where it acts as a urethral anchor. Alternatively, proximal anchor 14 may be embedded inside the tissue of the prostate gland PG or surrounding structures as outlined above. Distal anchor 12 and proximal anchor 14 are implanted in the anatomy such that a desired distance or tension is created in the connector 16. This causes the distal anchor 12 and the proximal anchor 14 to retract or compress a region of the prostate gland PG to relieve the urethral constriction. In FIG. 2, two implants 10 are implanted in a lateral lobe (side lobe) of prostate gland PG. The various methods, systems, and devices disclosed herein may be used to treat a single lobe or multiple lobes, including one or more lateral lobes and the median lobe, of the prostate gland or other anatomical structures, by deploying one or more implants to the targeted tissue.

The implants may be deployed at particular angles relative to the axis of the urethra to target one or more lateral lobes and/or median lobe of the prostate gland. For example, implant 10 may be deployed between the 1 o'clock and 3 o'clock position relative to the axis of the urethra to target the left lateral lobe of the prostate gland. In another example, implant 10 may be deployed between the 9 o'clock and 11 o'clock position relative to the axis of the urethra to target the right lateral lobe of the prostate gland. In another example, implant 10 may be deployed between the 4 o'clock and 8 o'clock position relative to the axis of the urethra to target the middle lobe of the prostate gland.

Examples of the implantation process may generally involve advancing the distal end of the tubular elongate member containing or configured to receive a coaxial delivery needle distally through the urethra of a subject toward the urinary bladder until the distal end reaches the prostatic urethra, adjacent one or more lobes of the enlarged prostate gland targeted for compression. In some examples, the targeted lobe of the prostate gland may be chosen while the device extends through at least a portion of the prostatic urethra. In other embodiments, the targeted lobe is identified prior to the procedure, for example via ultrasound imaging. A distal portion of the elongate member may, in some examples, be advanced into the bladder, where it may be positioned and/or rotated as needed to deploy the implant as desired within the targeted lobe upon retracting the elongate member until its distal end returns to the prostatic urethra.

The distal end of the needle may be advanced through and beyond the distal end of the elongate member, for example through the sidewall opening defined near the distal end of the elongate member. The needle may be made of nitinol in some examples, and its inner lumen may extend the entire length of the needle, from its proximal end to its distal end. The needle may curve and extend substantially orthogonally or perpendicularly away from the longitudinal axis of the elongate delivery member, for example in the manner shown and described in U.S. Pat. No. 11,298,115, the entire contents of which are incorporated by reference herein. Additional embodiments may involve advancing the needle through an opening defined by the distal end of the elongate member. Such embodiments may or may not define a sidewall opening. After exiting the elongate member, the clinician continues to advance the needle distally until its distal tip pierces through the urethral wall, targeted prostatic lobe, and outer prostatic capsule. Advancement of the needle may be driven by manual activation of an actuator, for example via trigger pull.

After the distal anchor member contained within the delivery needle is positioned distally beyond the outer surface of the prostatic capsule, the needle may be retracted proximally toward the urethra, where the elongate member remains, while the distal anchor member and middle portion are not retracted. As the needle is retracted, the distal anchor member and middle portion are unsheathed in a distal-to-proximal direction, such that the distal anchor member of the implant is unsheathed first, outside the prostatic capsule. Free from the constraints of the inner lumen of the delivery needle, the distal anchor member of the implant may assume an unconstrained anchoring configuration, which may comprise an orientation transverse to the longitudinal axis of the needle and middle portion, thereby anchoring the implant to the capsular side of the lobe.

Continued retraction of the needle unsheathes the middle portion of the implant within the lobe. The needle is further retracted until its distal tip is retracted through the urethral side of the lobe. Once the needle is fully retracted from the lobe and tension is applied to the middle portion implanted therein, the proximal anchor member or UE of the implant may be attached to the middle portion at the urethral side of the lobe.

In some embodiments, once the distal anchor member and attached middle portion have been deployed, with the needle retracted and middle portion tensioned, the anchor member may be pushed or seated by one or more components of the delivery device such that it captures the middle portion transverse to the anchor axis. The middle portion may then be cut just proximal to the anchor member to allow removal of the excess middle portion not positioned within the prostatic lobe.

Prior to insertion of any components of the delivery system, the implant recipient may undergo a regimen of antibiotics. Local anesthesia can be employed for the interventional procedure. A combination of an oral analgesic with a sedative or hypnotic component can be ingested by the subject. A topical anesthesia such as lidocaine liquids or gel can be applied to the bladder and urethra.

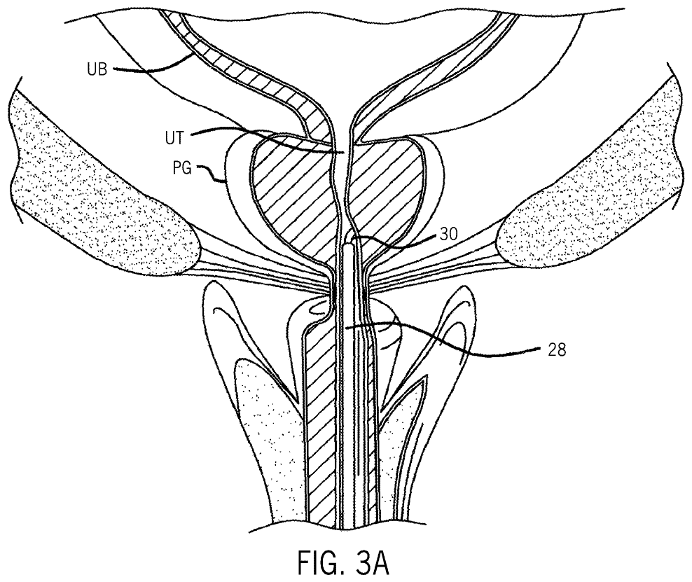

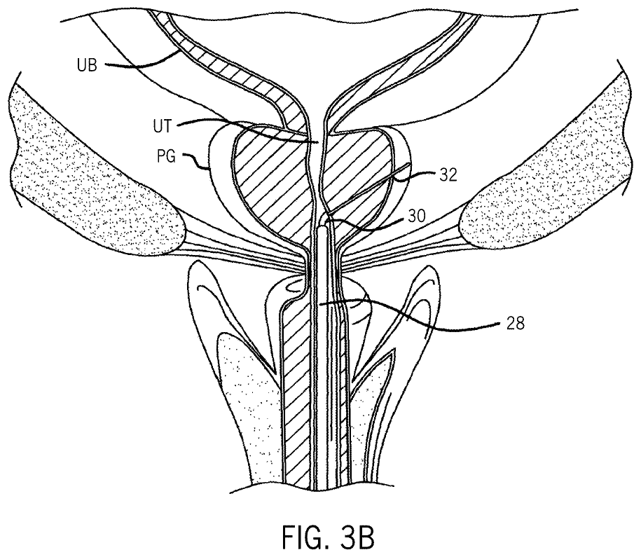

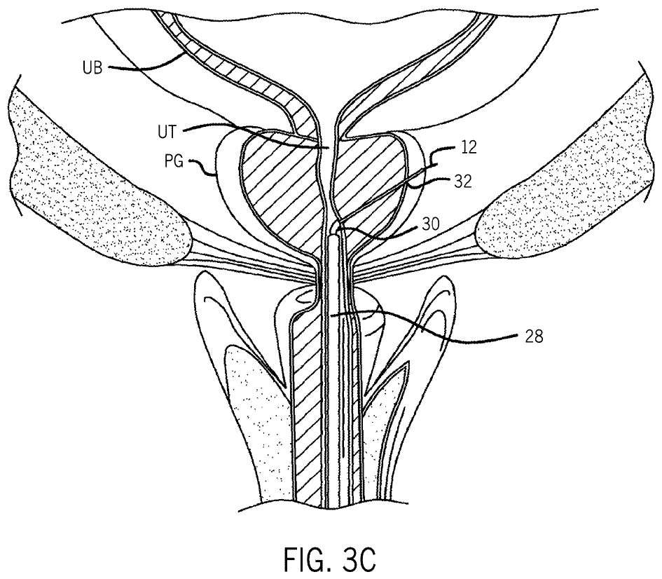

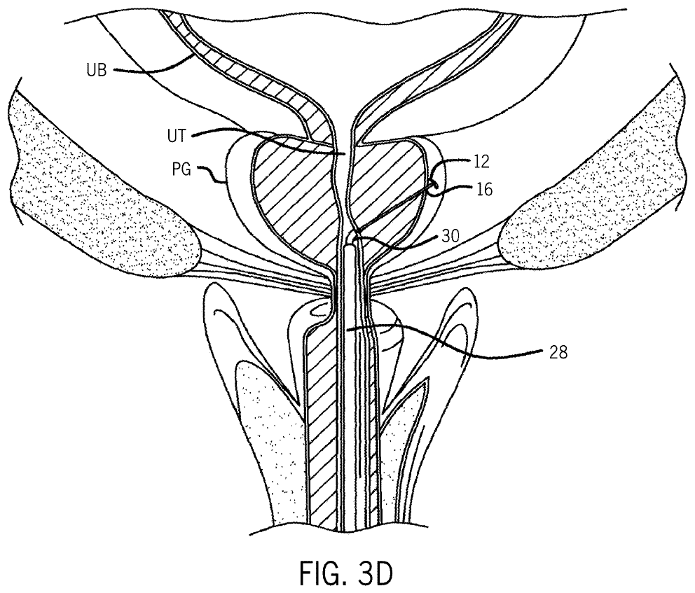

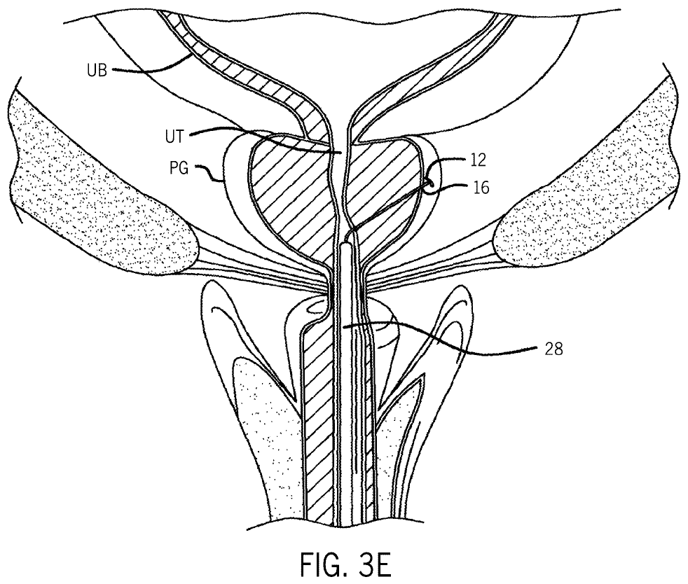

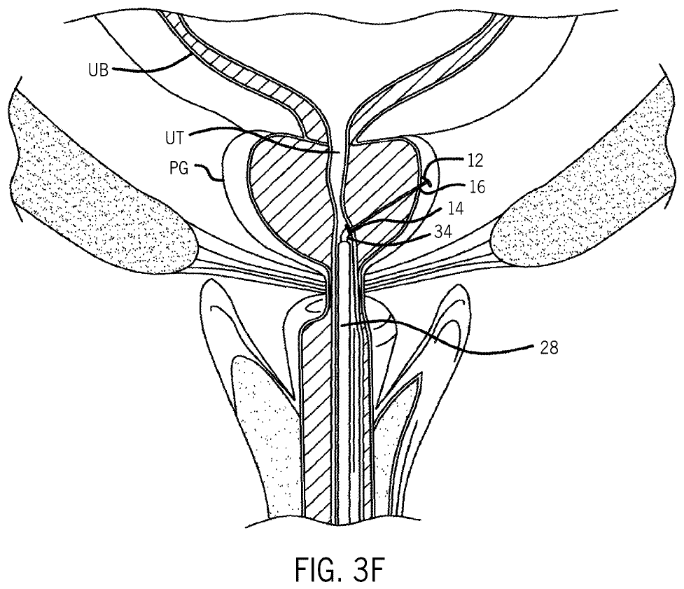

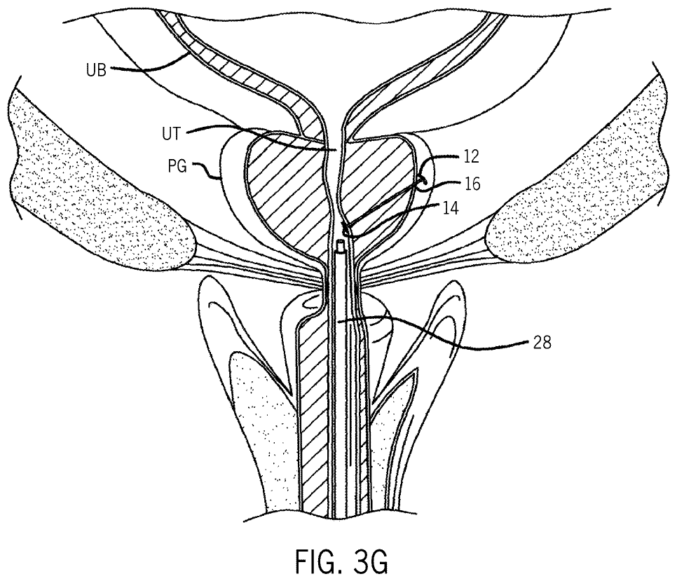

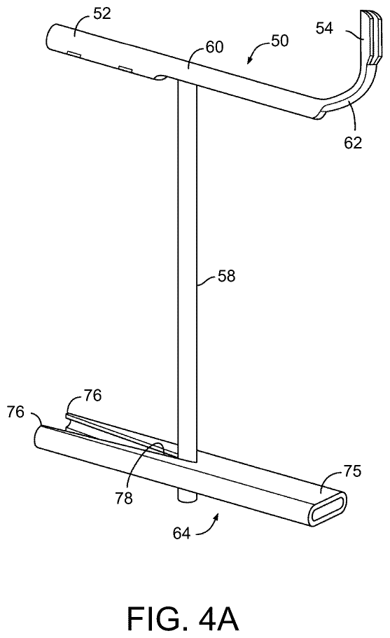

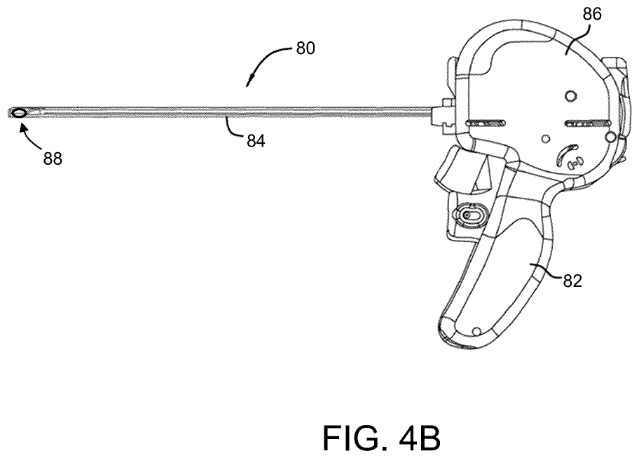

FIGS. 3A-3G show various steps of a specific, non-limiting example of a method of treating a prostate gland using one or more of the implants disclosed herein (see e.g., FIG. 4). One or more of the illustrated steps may be modified or excluded in accordance with certain embodiments disclosed herein, for example those not requiring a sheath. One or more of the depicted steps, shown for illustrative purposes, may be performed in a different manner depending on the particular delivery device(s) used, embodiments of which are disclosed herein. Similar methods may also be implemented to deploy retainers, implants, or compression devices in other anatomical structures.

In the step shown in FIG. 3A, a sheath 28, e.g., introducer sheath, which may comprise a standard resectoscope sheath, may be introduced into the urethra (trans-urethrally). The sheath 28 may be advanced through the urethra UT such that the distal end of the sheath 28 is positioned near a region of the urethra UT that is obstructed by a hypertrophied prostate gland PG. A distal anchor delivery device 30 featuring an elongate member or shaft assembly (the terms “elongate member” and “shaft assembly” used interchangeably herein) may then be introduced and advanced through an elongate inner lumen of the sheath 28. The distal anchor delivery device 30 may be placed in the sheath 28 after the distal end of the sheath 28 is positioned near the region of the urethra UT that is obstructed, or the distal anchor delivery device 30 may be pre-loaded in the sheath 28 before positioning of the sheath 28. Distal anchor delivery device 30 may be advanced through the sheath 28 such that the distal end of distal anchor delivery device 30 emerges out of the distal end of the sheath 28. The distal anchor delivery device 30 may be oriented such that a working channel opening of the distal anchor delivery device 30 points toward a lateral lobe of the prostate gland PG.

In the step shown in FIG. 3B, a penetrating member or needle 32 may be introduced through the distal anchor delivery device 30. The needle 32 may be placed in the distal anchor delivery device 30 after the distal anchor delivery device 30 is advanced through the sheath 28, or, alternatively, the needle 32 can be pre-loaded in the distal anchor delivery device 30. In one non-limiting embodiment, the needle 32 is a 20-gauge needle. The needle 32 is advanced through the distal anchor delivery device 30 such that it emerges through the working channel opening. The needle 32 may be further advanced until it penetrates through the tissue of prostate gland PG and the distal end of the needle 32 emerges out of the capsule of the prostate gland CP.

In the step shown in FIG. 3C, the distal anchor 12 connected to the connector 16 may be deployed at, or just beyond, the outer surface of the prostatic capsule. In some embodiments, the distal anchor 12 may be deployed by being extended beyond the distal end of the needle 32. In other embodiments, the distal anchor 12 may be deployed by being held in place by a pusher or connector while the needle 32 is retracted, thus exposing the distal anchor and unsheathing the distal anchor 12 and connector 16 upon continued needle retraction. The distal anchor 12 can be pre-loaded within the needle 32, or it can be loaded into the needle 32 after the needle 32 has been advanced through the distal anchor delivery device 30.

In the step shown in FIG. 3D, the needle 32 may be removed from the distal anchor delivery device 30 by pulling the needle 32 in the proximal direction. In the step shown in FIG. 3E, the distal anchor delivery device 30 may be removed from the sheath 28 by pulling the distal anchor delivery device 30 in the proximal direction. Also, the connector 16 may be pulled to orient the distal anchor 12 approximately perpendicularly or otherwise transverse to the connector 16, against the outer surface of the prostatic capsule.

In the step shown in FIG. 3F, the connector 16 may be passed through the proximal anchor 14 located on a proximal anchor delivery device 34. The proximal anchor delivery device 34 may be advanced through the sheath 28 such that the distal end of the proximal anchor delivery device 34 emerges out of the distal end of sheath 28. A desired tension may be introduced into the connector 16 such that distal anchor 12 is pulled by the connector 16 with a desired force to compress the prostate tissue. The proximal anchor 14 can be visualized through an endoscope or under fluoroscopy and advanced along the connector 16 until the desired retraction of the tissue is achieved. In other embodiments, the proximal anchor 14 may be a v-shaped or clothespin-shaped piece that may be forced, in some cases at high speed, onto the connector 16 to fixedly engage the connector.

In the step shown in FIG. 3G, the connector 16 is attached to the proximal anchor 14 and the surplus or residual connector extending proximally from the proximal anchor 14 is cut. The proximal anchor 14 is also released from the proximal anchor delivery device 34, thus deploying proximal anchor 14 and the implant 10 as a whole in the targeted anatomy. The anchor delivery device 34 and the sheath 28 may then be removed from the anatomy, retracting proximally through the urethra.

This method, and variations thereof, may be used to retract, lift, support, reposition or compress multiple regions or lobes of the prostate gland PG. In the particular example shown in FIGS. 3A-3G, the distal anchor 12 is deployed on the outer surface of the prostatic capsule. Thus, the distal anchor 12 acts as a capsular anchor. Alternatively, distal anchor 12 may be deployed inside the tissue of prostate gland PG or beyond the prostate as outlined previously. Similarly, in the method shown in FIGS. 3A-3G, the proximal anchor 14 may be deployed on the inner wall of urethra UT, acting as a urethral anchor. Alternatively, proximal anchor 14 may be deployed inside the tissue of prostate gland PG.

Embodiments disclosed herein may involve repeating the implant deployment process (or one or more steps thereof) without having to remove the elongate member or elongate shaft assembly from the urethra, as embodiments of the disclosed implant delivery devices may be reloaded with cartridges containing one or more implants (without an elongate member), and other embodiments may include one or more subassemblies or mechanisms configured to simultaneously store and serially deploy multiple implants. Accordingly, after insertion of the elongate shaft assembly into a urethra of a patient, multiple implants may be deployed simply by activating one or more manually engageable actuators one or more times until the necessary number of implants have been deployed. In embodiments featuring removable cartridges, serial implant deployment may simply involve exchanging the necessary number of cartridges and activating one or more manually engageable actuators until the necessary number of implants have been deployed.

FIG. 4A provides a perspective view of a non-limiting example of an anchor assembly or implant deployed using the systems, devices, and assemblies herein. In an unconstrained configuration, the distal anchor component 50 (or “CT”) may include a head portion 52, which may be generally orthogonally oriented with respect to a tail portion 54. While housed in the distal portion of a delivery needle and prior to deployment at a target area, the distal anchor component 50 may be constrained to a generally straight configuration relative to the longitudinal axis of the needle, only subsequently assuming the unconstrained (i.e., orthogonally oriented) configuration upon deployment from the needle assembly.

In certain embodiments, the distal anchor component 50 may be formed from a nitinol base stock that is generally tubular and can be shape-set to include the orthogonally oriented configuration of the head portion 52 with respect to the tail portion 54. A suture 58 may be attached to the distal anchor component 50. In one embodiment, a polyethylene terephthalate (PET) suture portion 58 is thermoformed onto locking features in the distal anchor component 50. The distal anchor component 50 may be locally heated to re-flow the suture onto the end of the distal anchor component 50 and into cutouts on the distal anchor component 50. The distal anchor component 50 may be attached to the suture portion 58 through any of several known techniques for bonding a PET material to a nitinol material.

In one embodiment, a mid-section 60 of the distal anchor component 50 provides a structural transition from the head portion 52 to the tail portion 54 and has a portion of a side wall removed in the area of mid-section 60. A further portion of the side wall is removed to define a connector section 62 of the tail portion 54 which extends from the mid-section 60. In one embodiment, this connector section 62 may include a bend that creates the orthogonally oriented configuration. Thus, in its pre-implanted form, the anchor assembly can include a distal anchor component 50 whose initial engagement with a suture portion 58 is generally coaxial.

Still referring to FIG. 4A, in one embodiment the proximal anchor component 64 (or “UE”) includes prongs 76 that grip the suture portion 58. The interior structure of the prongs 76 may function to disrupt the surface of the suture portion 58, both pressing into the suture portion 58 and compressing the suture portion 58 therebetween. A tab 78 may be included, extending from one or more of the prongs 76 to help create secure engagement between the proximal anchor component 64 and the suture portion 58. In some examples, the proximal anchor component 64 may comprise stainless steel. In some embodiments, the implant in its entirety may comprise a polymer composition, for example resembling a “T” in whole or in part.

In certain embodiments, the proximal anchor component 64 may be present in the shaft assembly of a delivery device in a configuration that is separate and disconnected from the distal anchor component 50 and the suture portion 58, which may be coupled, attached, or otherwise engaged with each other and contained within the needle assembly. After the distal anchor component 50 and the suture portion 58 have been placed within the targeted tissue, the proximal anchor component 64 may be securely engaged with the suture portion 58 to form the fully assembled anchor assembly or implant. To facilitate engagement of the proximal anchor component 64 with the suture portion 58, the proximal anchor component 64 may include, in some examples, a rigid, generally cylindrical back end 75. This rigid, generally cylindrical back end 75 can be used to push the proximal anchor component 64 into engagement with the suture 58 via transfer of the mechanical energy in the handle assembly of the associated delivery device.

The tissue approximation anchors disclosed herein and shown in FIG. 4A may be designed to be useable in a physician's clinical office environment (in contrast to requiring a hospital environment) with a delivery tool. The delivery tool may be used with a 19 F or 20 F sheath in some examples. Additionally, the material selection and construction of the tissue approximation anchor still allows for a subsequent TURP procedure to be performed, if necessary, on the prostate. In this suture-based, tissue approximation technique, a needle delivery mechanism may be used to implant an anchor assembly.

Implants disclosed herein may be delivered to a targeted lobe of a prostate gland using a delivery system that further includes a delivery device comprising a tubular elongate member (or shaft assembly) and at least one hollow delivery needle configured to be advanced therethrough. The needle may have a sharp distal tip configured to pierce the prostate gland, including the outer capsule, along with an inner lumen configured to receive and house a distal anchor member (or CT) and a suture (or other middle portion or connector of an implant).

Examples of a delivery device may generally include a handle assembly supporting an elongate portion comprising a tubular elongate member or shaft assembly. The elongate member may be substantially rigid or flexible and defines a low profile suited to navigate body anatomy to reach an interventional site. Examples of the elongate member may define two or more inner lumens, with one lumen configured to accommodate extension of the distal anchor member and middle portion therethrough. Substructure may be provided to maintain a longitudinal profile of the elongate member so that an interventional procedure can progress as intended. Embodiments of the delivery device may also include an endoscope, providing the ability to view the interventional procedure, which may be positioned within an elongate scope tube within the elongate member. The elongate member may be sized to fit within a cystoscopic sheath for patient tolerance during a procedure in which the subject is awake rather than under general anesthesia. Non-limiting examples of the sheath may be 19 F or 20 F. Using the disclosed systems, insertion of one or more implants in a prostate gland may be performed in an outpatient setting.

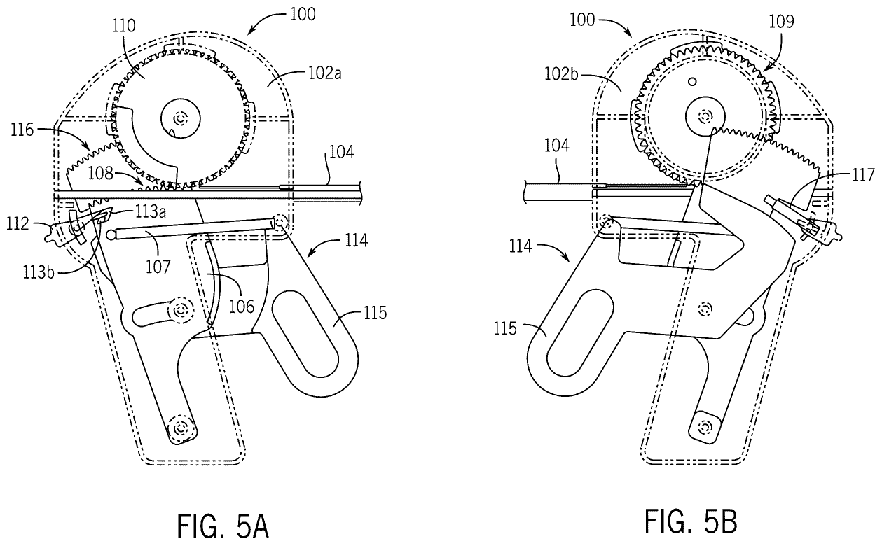

FIG. 4B illustrates one non-limiting example of a delivery device 80 having structure configured to gain access to an interventional site and deploy a prostatic implant, such as implant 10. As shown, the delivery device 80 may include a handle assembly 82 connected to an elongate shaft assembly 84, which may surround an elongate delivery needle defining an inner lumen and arranged coaxially to the elongate shaft assembly 84. Various delivery device embodiments disclosed herein may include a handle assembly and elongate shaft assembly, with different configurations of each. For example, delivery devices disclosed herein may have different means of actuation than the delivery device 80 shown in FIG. 4B. Examples may include at least one trigger, as shown for example in FIGS. 5A and 5B. One or more additional features may be included, or omitted, relative to the example of delivery device 80, which is shown for illustrative purposes.

Delivery devices disclosed herein, such as delivery device 80, may further include a number of subassemblies configured to deliver and employ one or more implants at a target site. A handle case assembly 86, including handle parts that form part of the handle assembly 82, is also included. The handle assembly 82 is sized and shaped to fit comfortably within an operator's hand and can be formed from conventional materials. Windows can be formed in the handle case assembly 86 to provide access to internal mechanisms of the device so that a manual override is available to the operator in the event the interventional procedure needs to be abandoned.

In some examples, the elongate shaft assembly 84 may define at least one inner lumen sized and configured to accommodate longitudinal insertion of at least the hollow delivery needle and prostatic implant therethrough, with the distal anchor member or CT of the implant nestled within the delivery needle, and the proximal anchor component or UE included in the elongate shaft assembly 84 in a delivery configuration, in some embodiments. In some examples, the CT and middle portion or suture may be enclosed within a first lumen of the elongate shaft assembly 84, and the UE may be enclosed within a second lumen of the elongate shaft assembly 84. The elongate shaft assembly 84 may have a shape and/or flexibility configuring it to navigate through a urethra without kinking or puncturing the urethral wall. In some examples, the elongate shaft assembly 84 may be substantially rigid, such that it maintains an approximately straight configuration during its insertion through the urethra. According to such examples, the distal portion of the elongate shaft assembly 84 may be angled toward or away from various anatomical features surrounding the urethra, e.g., one or more lobes of the prostate gland, by adjusting the angular orientation of the proximal end of the elongate shaft assembly 84 outside the body. The distal end of the elongate shaft assembly 84 may comprise smooth, blunt, and/or beveled surfaces to avoid puncturing the urethral wall. Embodiments may include a sidewall opening 88 (or exit port) defined by a distal portion of the elongate shaft assembly 84. The sidewall opening 88 may be sized and configured to accommodate passage of the hollow delivery needle therethrough.

In some examples, the handle assembly 82 may simultaneously contain two or more implants, e.g., two, three, four, five, six, seven, eight, nine, ten implants, or more. Such embodiments may include internal subassemblies configured to deploy the implants in serial fashion. In some examples, a delivery device may include a handle assembly and a removable cartridge configured to couple therewith. According to such examples, each removable cartridge may contain one implant or portion thereof, e.g., CT and/or suture.

Forming one or more devices, device components, and device assemblies disclosed herein may involve one or more molding processes, metal-working, and/or multi-part assembly. Plastic components of the various devices can be injection molded in some embodiments. Metal components may be formed via stamping processes. Laser welding may be employed to fix metal components to each other. A variety of connector components and tools, e.g., snaps, screws, etc., may be used to assemble a given device.

Some embodiments of a delivery device disclosed herein may include at least one internal spooling mechanism configured to store and deliver one or more CT/suture assemblies to a target site in response to manual actuation of the delivery device, for example via trigger pull. Examples of the delivery device may include a handle assembly and a removable cartridge, the two components configured to interact via one or more subcomponents or assemblies, such as gears, to translate manual actuation of the delivery device (e.g., via trigger pull) into the insertion of a delivery needle through a targeted tissue and the subsequent implantation of each CT/suture assembly. Embodiments of the cartridge may be loaded into the side or rear of the delivery device during a procedure without removing the elongate shaft assembly (or elongate member) of the delivery device from the subject, such that multiple CT/suture assemblies can be implanted during the procedure by simply exchanging cartridges and activating the delivery device as many times as necessary to sufficiently retract the enlarged prostatic tissue causing urethral impingement. Additional embodiments of a delivery device may include one or more spooling mechanisms fixed or seated within a handle assembly, in lieu of a cartridge. Examples of such devices may be configured to simultaneously contain multiple implants delivered successively during a single procedure. Successive or serial deployment of multiple implants that are simultaneously contained in a delivery device may further involve one or more ratcheting and/or indexing mechanisms implemented to drive and control the deployment of each implant. As shown and described in connection with the following embodiments, various components of the delivery devices may be directly, indirectly, and/or operatively coupled to deploy the implants.

FIG. 5A provides a partially transparent view of the right side of a portion of a handle assembly 100 included in an embodiment of a delivery device disclosed herein. FIG. 5B provides a partially transparent left side view of the same device, which may be configured to deploy multiple implants to a targeted tissue during a treatment procedure. The device may include or be coupled with one or more internal spooling assemblies and associated mechanisms together configured to deploy the implants. As shown, the handle assembly 100 may include a right handle case component 102a attached to a left handle case component 102b (both shown in dashed lines to indicate transparency in the illustrated representations), together defining an internal cavity. An elongate shaft assembly 104 extends longitudinally from the handle assembly 100. The shaft assembly 104 may comprise a tubular member defining a low profile (e.g., having a cross-sectional width or diameter suitable for insertion into the patient's urethra) and an inner lumen through which one or more implants and a needle used to deliver them may be advanced during a procedure. The shaft assembly 104 may also include an elongate scope tube configured to receive a viewing device, e.g., endoscope. Embodiments of the shaft assembly 104 may include components or subassemblies configured to deliver and seat a UE on an implanted suture. The shaft assembly 104 may thus comprise a multi-component assembly in some embodiments. During a treatment procedure, the shaft assembly 104 may be advanced distally through the urethra until its distal end reaches the prostatic urethra, adjacent one or more targeted lobes (lateral or medial) of the prostate gland. In some examples, the distal end of the shaft assembly 104 may be extended to, and within, the bladder, after which it may be retracted proximally into the prostatic urethra, for example as or after the shaft assembly 104 is rotated or otherwise repositioned as necessary to deliver the implant(s).

As further shown, the handle assembly 100 may also include a needle actuator 106, which may comprise a trigger or lever, coupled with an internal needle spring 107 or spring structure or other biasing component or mechanism. The spring-loaded needle actuator 106 may include or define needle actuator gear teeth 108 configured to engage complementary gear teeth 109 included or defined by a needle spool member 110 coupled with a needle used to pierce through prostatic tissue with a CT/suture assembly in tow. A spring-loaded lever 112 (e.g., a safety) may protrude from the rear of the handle assembly 100, holding the needle actuator 106 in an energized state until being released via disengagement of an internal latch 113a with a raised portion 113b of the needle actuator 106 in response to manual engagement with the externally protruding portion of the lever 112. A suture actuator 114, which may comprise a trigger or lever defining a manually engageable portion 115 and internally positioned suture trigger gear teeth 116, is also included. The suture actuator 114 may be spring-loaded via an internal suture spring 117 or spring structure. In response to manual activation, the components of the handle assembly 100 are configured to transfer mechanical energy to a removable cartridge carrying one or more prostatic implants to deploy the implant(s) into target tissue.

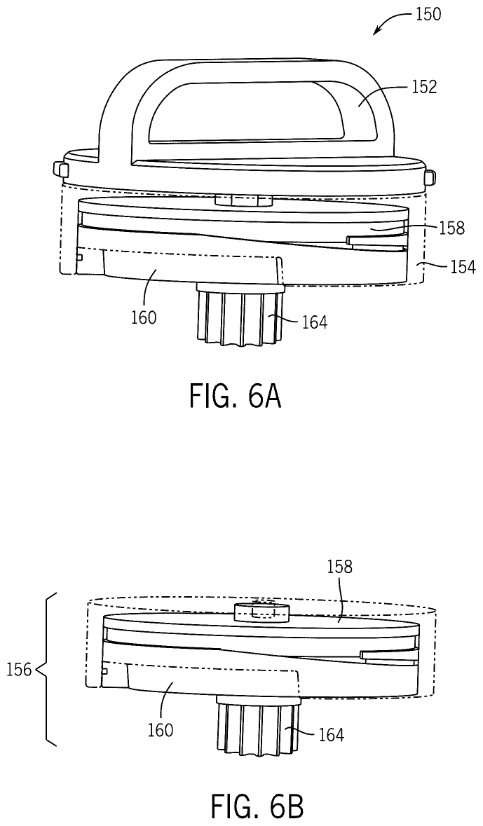

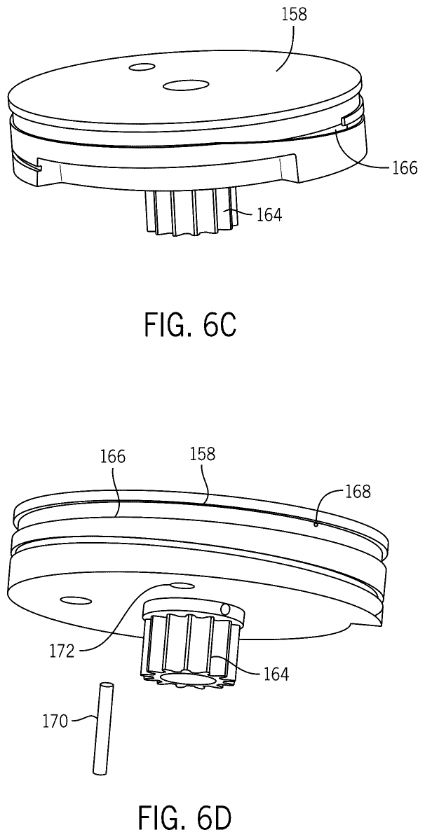

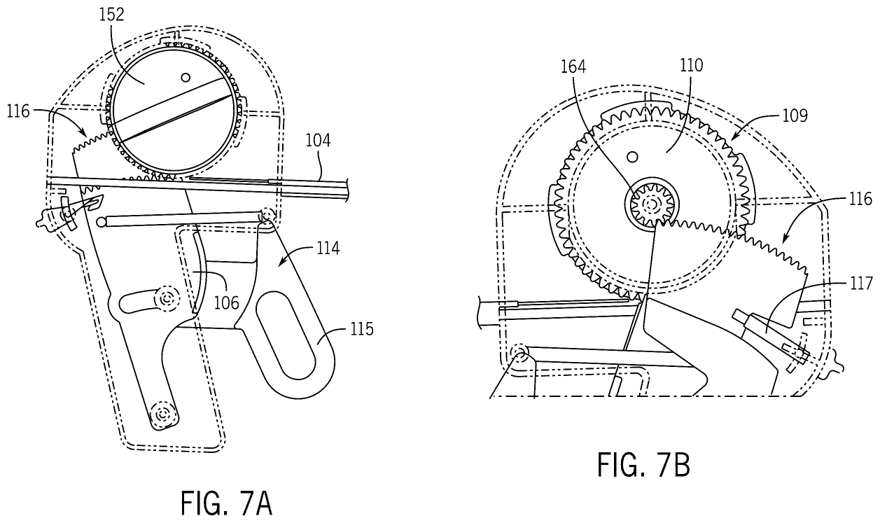

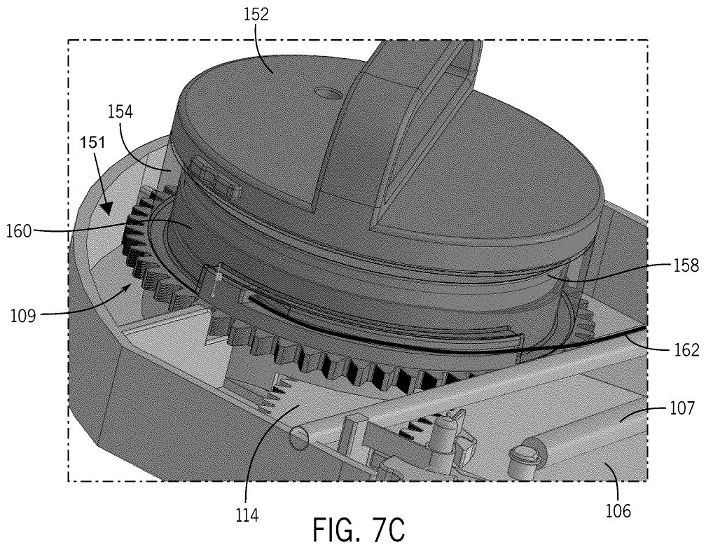

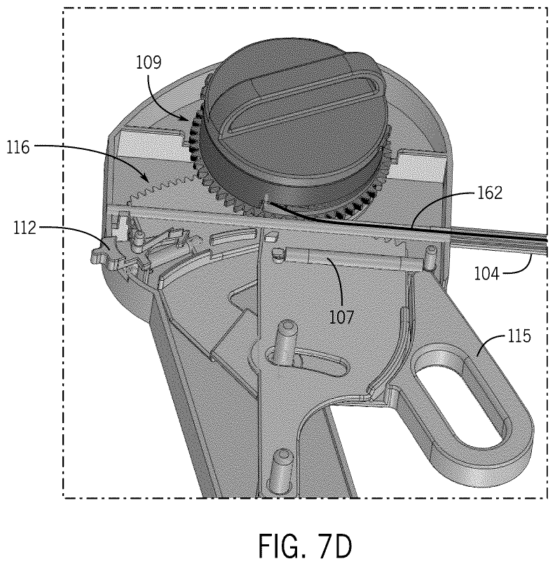

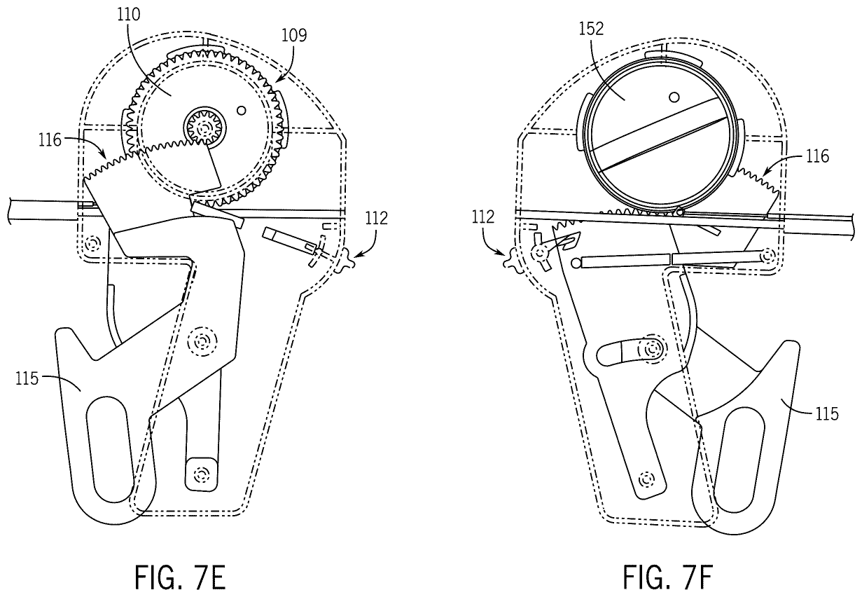

FIGS. 6A-6D illustrate an example of a removable cartridge 150 configured to couple with the handle assembly 100, and FIGS. 7A-7F show the removable cartridge 150 coupled to the handle assembly 100. In the example shown, the cartridge 150 includes or is coupled with a knob 152 configured to lock the cartridge 150 to the handle assembly 100. The knob 152 may be coupled with a housing 154 (shown in transparency) configured to constrain a CT/suture assembly wrapped around a suture spool assembly 156, which may include a suture spool member 158 coupled to a suture track member 160. The suture track member 160 may be keyed to an elongate tissue penetrating member defining an inner lumen, such as a needle (see, e.g., needle 162 in FIGS. 7C and 7D). The suture spool member 158 may include or define a spool gearing portion 164, and the suture track member 160 may define a circumferential track 166 configured to guide the CT (and suture attached thereto) into the needle 162 upon rotation of the suture spool assembly 156.

In embodiments, a suture may wrap around the suture spool member 158 and then into the track 166 defined by the suture track member 160. The suture spool member 158 may be configured to rotate relative to the suture track member 160, such that rotating the suture spool member 158 unspools the suture through the track 166 of the suture track member 160 and into the needle 162. The suture may be connected to the spool member 158 at a connection point 168, from which it may wrap around the spool member 158 one or more times, e.g., approximately twice, before advancing into the track 166, with the housing 154 preventing the suture from unraveling away from the surface of the spool assembly 156. A coupling member 170, such as a pin, may extend through a complementary receiving slot 172 that extends through at least a portion of the spool member 158 and track member 160, thereby keeping the two components from separating.

Before or during a procedure, the cartridge 150 may be coupled with the handle assembly 100, for example by inserting the cartridge 150 into a cartridge bay or cavity 151 defined by the handle assembly 100, where it may then be locked into place by a user, for example by turning the knob 152 from a first, unlocked configuration to a second, locked configuration. Before manual activation of the delivery device to deploy an implant, the suture actuator 114 may be arranged in its non-energized state, with the manually engageable portion 115 away from the handle body, and the needle actuator 106 may be held back in its energized state via the lever 112. The suture may be spooled around the suture spool assembly 156, with the CT positioned toward the end of the track 166, furthest from the connection point 168 defined by the suture spool member 158.

Upon coupling and locking the cartridge 150 with the handle assembly 100, the spool gearing portion 164 may engage with the suture actuator gear teeth 116, such that manual activation of the suture actuator 114 (e.g., squeezing) translates linear movement of the suture actuator gear teeth 116 into rotational movement of the suture spool member 158 relative to the suture track member 160, thereby unwinding the spooled CT/suture. The suture track member 160 and housing 154 may key into the needle spool member 110, such that the two components also move together upon activation. After coupling the cartridge 150 and handle assembly 100, the track 166 may lead directly into the lumen of the delivery needle 162, such that unwinding of the CT/suture in response to activation of the suture actuator 114 causes the CT/suture to advance into the proximal end of the needle 162.

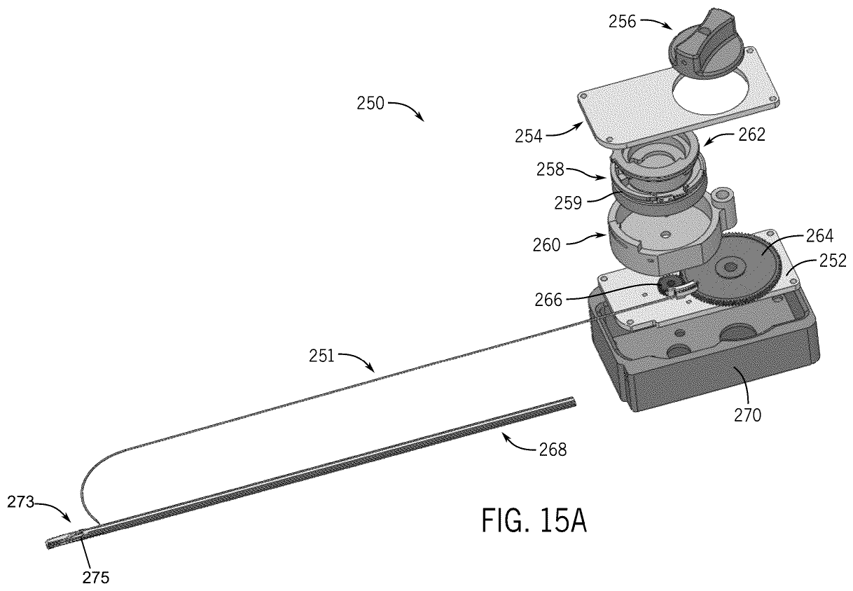

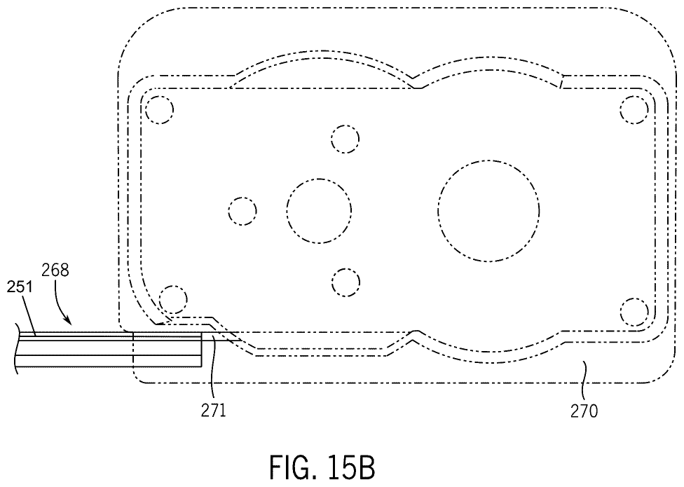

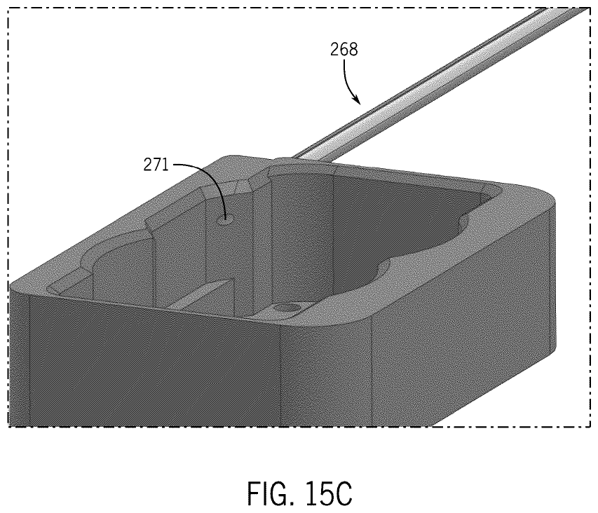

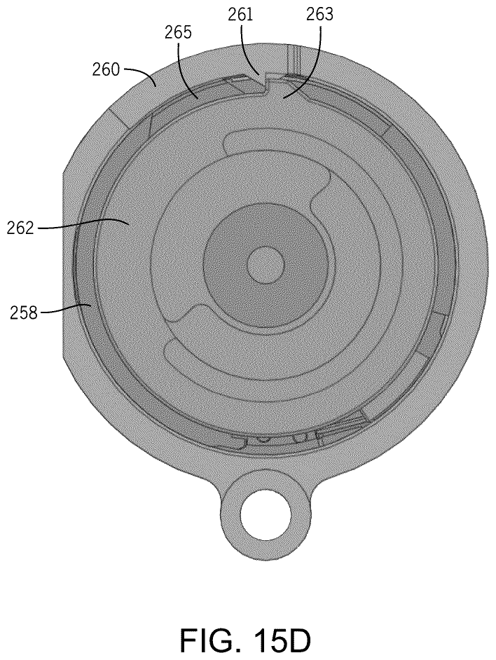

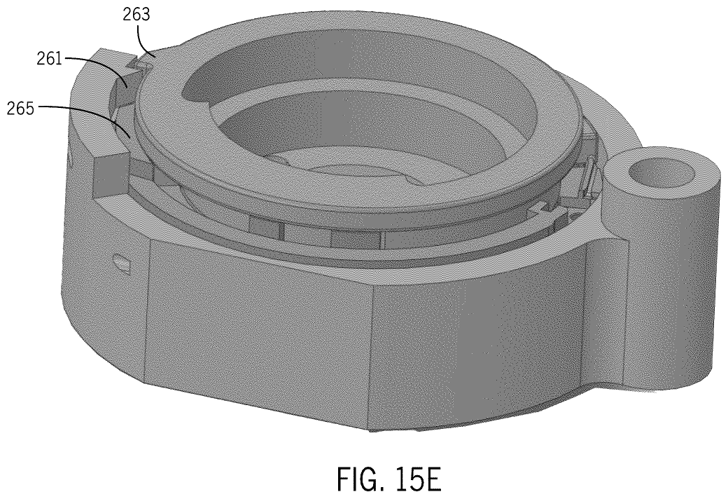

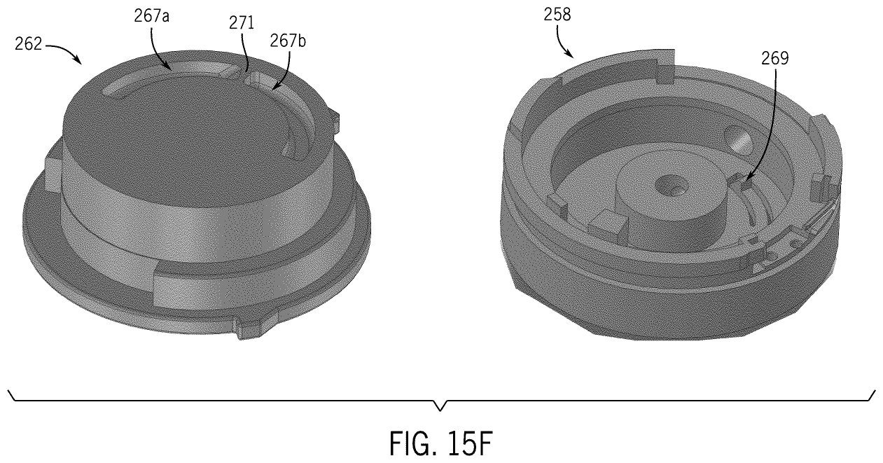

In operation, a user may first engage (e.g., press) the lever 112 at the back of the handle assembly 100, disengaging the internal latch 113a from the raised portion 113b and releasing the spring-loaded needle actuator 106, which then fires forward, toward the shaft assembly 104. Forward motion of the needle actuator 106, and thus the needle actuator gearing 108, drives rotation of the needle spool member 110, advancing the needle 162 through the shaft assembly 104 until the needle 162 extends through and beyond the distal end of the shaft assembly 104, for example via an exit port defined by the assembly (see e.g., exit port 275 in FIG. 15A), which is positioned within the prostatic urethra. The needle 162, a portion of which may have a defined radius of curvature, may then penetrate and advance through the targeted lobe until its distal tip pierces through the prostatic capsule. Accordingly, the needle 162 may be deployed or fired distally via manual engagement with a component of the device, such as lever 112, which transitions the needle drive mechanism, featuring needle spring 107, from a loaded configuration to an unloaded configuration.

To deliver the CT/suture assembly distally through the needle 162, the user may engage or manipulate (e.g., squeeze) the manually engageable portion 115 of the suture actuator 114, thereby rotating the spool gearing 164 via movement of the suture actuator gear teeth 116 engaged therewith. This gearing mechanism may rotate the spool member 158 approximately two revolutions, unspooling the CT/suture through the track 166, into and through the needle 162 previously advanced through the shaft assembly 104, and toward the prostatic urethra. The CT/suture may thus be fired or deployed via a single activation (e.g., trigger pull) of the suture actuator 114. In this regard, the implant deployment process and associated delivery device may be considered a “single trigger pull” system.

In some examples, the CT/suture may be unsheathed at the implantation site by retracting the needle 162 in the proximal direction, back toward the distal end of the shaft assembly 104 positioned within the urethra. According to such embodiments, the CT may come to a stop just before reaching the distal bevel of the needle 162, which may be positioned beyond the outer surface of the prostatic capsule. In other examples, where the CT is pushed beyond the distal bevel of the needle 162 at the outer surface of the prostatic capsule, the CT may come to a stop after it has been fully advanced through the distal needle tip. During either of these deployment processes, the user may hold the suture actuator 114 in a second position, e.g., squeezed, relative to the initial resting position, as shown for instance in FIG. 5A.

While continuing to hold or squeeze the suture actuator 114, the user may engage the needle actuator 106 until the raised portion 113b is re-engaged by the internal latch 113a of the lever 112. Squeezing the needle actuator 106 rotates the needle spool member 110 in the opposite direction relative to the deployment step, which retracts the needle 162 back into the shaft assembly 104 and, in some examples, unsheathes the CT/suture. After retraction of the needle 162 into the shaft assembly 104, the CT/suture assembly may be fully implanted, but before attaching the UE to the suture at the urethral side of the lobe, the CT/suture must be tensioned in some examples. For this, the user may gently release the suture actuator 114, thereby pulling the CT and suture in the proximal direction until the suture is tensioned through the prostatic tissue and the CT is substantially flush with the outer surface of the prostatic capsule, transverse to the longitudinal axis of the suture. The force of tensioning may be determined by the properties of the suture spring 117.

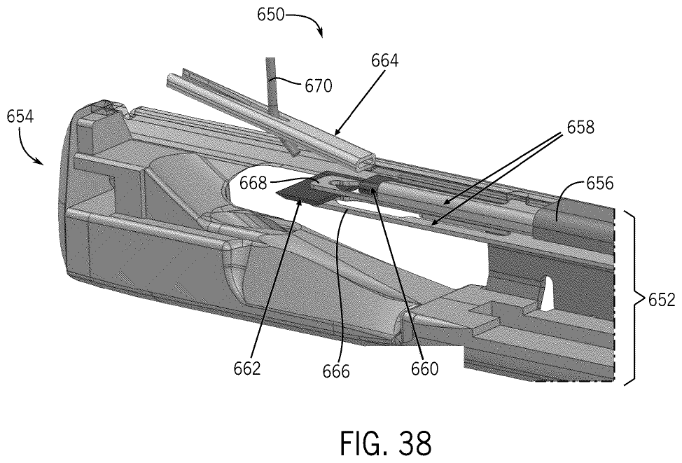

The user may then activate the mechanisms through which the UE is seated on the suture and the surplus suture cut. Non-limiting examples of such mechanisms are described in U.S. Pat. No. 11,298,115, the entire contents of which are incorporated by reference herein, along with the additional mechanisms described below, for example in connection with FIGS. 38-48R.

When the suture is cut, the suture spring 117 may pop or spring back to its original resting position (i.e., first, unloaded configuration), which may pull the remaining length of surplus suture back into the handle assembly 100, around the spool assembly 156. The cartridge 150 may then be unlocked via the knob 152 and removed from the handle assembly 100. At this point, the needle actuator 106 and suture actuator 114 have been reset to their original position, re-energized and ready to transfer mechanical energy to another cartridge in response to manual activation, thereby deploying another CT/suture assembly after coupling the handle assembly 100 with a new cartridge 150. The cartridge exchange process may be completed without removing the shaft assembly 104 from the urethra, such that the delivery device as a whole may remain stationary through multiple cartridge exchanges until completion of the procedure, which may involve deploying one or more implants, including two, three, four, five, six, seven, eight, nine, ten implants, or more. Serial deployment of multiple implants without removing the shaft assembly 104 from the urethra may significantly improve patient comfort, reduce or prevent unwanted tissue manipulation, decrease the procedure time, and allow for visualization to be maintained at all points during the procedure.

In some examples, the ability to leave the device in the subject throughout the procedure may also eliminate the need for an introducer sheath (e.g., sheath 28) that would otherwise be inserted through the urethra to provide a passageway for successive removal/reinsertion of the shaft assembly 104. This may reduce the effective diameter of the shaft assembly 104 necessary to perform the procedure, further improving patient comfort.

The components and mechanical interactions of the handle assembly 100 and cartridge 150 may also facilitate troubleshooting efforts, as one or more actuators (e.g., triggers, levers, knobs, switches, etc.) may be directly connected to the needle 162 and/or suture. For example, if the needle 162 needs to be withdrawn during a procedure for some reason, e.g., bone strike, it can be withdrawn by simply engaging the needle actuator 106. In a similar fashion, via engagement with the suture actuator 114, each CT/suture assembly can be directly controlled in response to various factors or complications that may arise during a given procedure, such as improper placement or slippage of the distal end of the shaft assembly 104.

As shown and described above, embodiments of the cartridge 150 may advantageously lack an elongate shaft assembly, needle, and/or one or more springs or actuators, one or more of which, including all, may be included in, or attached or fixed with the handle assembly 100 instead. This reduces the number of discrete parts present within the cartridge 150 relative to preexisting devices, along with the associated complexity of the delivery device as a whole. These benefits may not only reduce the cost of the cartridges and procedures in their entirety, but may increase the reliability of the device and procedure by minimizing the likelihood of malfunctions that could otherwise occur during the coordinated action of a variety of movable parts. One or more components of the device, including the handle assembly, case components, cartridge, and components thereof, may be formed via a molding process, e.g., injection molding. One or more components of the delivery device may be formed separately and subsequently coupled, attached, or fixed.

As shown in FIGS. 5A-7F and described above, delivery devices and systems disclosed herein can deploy multiple implants to a prostate gland of a human subject without removing the elongate shaft assembly of the delivery device from the subject. Embodiments of such devices/systems may include a handle assembly and a removable cartridge. The removable cartridge may be configured to contain all or a portion of at least one distal anchor component (CT) and a connector portion (e.g., suture) attached thereto. The distal anchor component may be configured for attachment to an outer surface of the prostatic capsule, and the suture may be configured to be placed within, and extend through, prostate tissue. The handle assembly may include or be attached to at least one actuator, a needle configured to receive and house the CT/suture and pierce through the prostate gland, and an elongate delivery shaft assembly configured to be advanced longitudinally through the urethra of the subject and accommodate advancement of the needle therethrough. The removable cartridge can be detached, decoupled, disengaged, or otherwise removed or separated from the handle assembly after insertion of the shaft assembly within the urethra and without removing the shaft assembly from the urethra. Embodiments of the delivery device can include at least one spooling mechanism, which may include multiple spools or spool members. For example, the spooling mechanism may include one or more spool members in the handle assembly and one or more spool members in the removable cartridge. The spool member(s) included in the handle assembly may comprise a needle spool, i.e., a spool member configured to couple with the needle of the delivery device. The spool member(s) included in the cartridge may include a suture spool, i.e., a spool configured to couple with the suture of a prostatic implant. The spool members may therefore couple with one or more components of a prostatic implant and/or delivery device configured to deliver the implant to a treatment site. A spooling mechanism can include one or more gear portions or features configured to translate engagement or activation of the actuator(s) into rotation of one or more spool members, such as the needle spool and suture spool, thereby driving deployment of the needle and CT/suture positioned therein. Actuators included on or in the delivery device can include an actuator configured to move the needle, i.e., a needle actuator, as well as an actuator configured to unwind, deliver and/or retract (e.g., tighten) the suture, i.e., a suture actuator. One or both of the needle actuator and suture actuator may be attached to a spring or other biasing component. In some examples, the needle actuator and suture actuator are attached to separate springs or biasing components. One or both of the needle actuator and suture actuator may include or define gearing portions or features, e.g., gear teeth. For instance, the needle actuator can include gear teeth configured to engage complementary gear teeth included on the needle spool member. Examples may further include a movable lever or other manually engageable component configured to hold or retain the needle actuator in an energized state until released via manual engagement (e.g., squeezing, pushing, or depressing) or disengagement (e.g., release). Disengagement of the lever or other manually engageable component of the needle actuator may cause the needle to unwind from the needle spool and advance distally through the shaft assembly, toward and through the targeted prostate tissue. Examples of the suture actuator may also include or define gearing portions or features, e.g., gear teeth. For instance, the suture actuator can include gear teeth configured to engage the suture spool member, such that activation or manual engagement with the suture actuator drives rotation of the suture spool member, thereby unwinding the suture coupled with the suture spool member. The suture can be advanced through the needle and prostate tissue upon activation of the suture actuator.

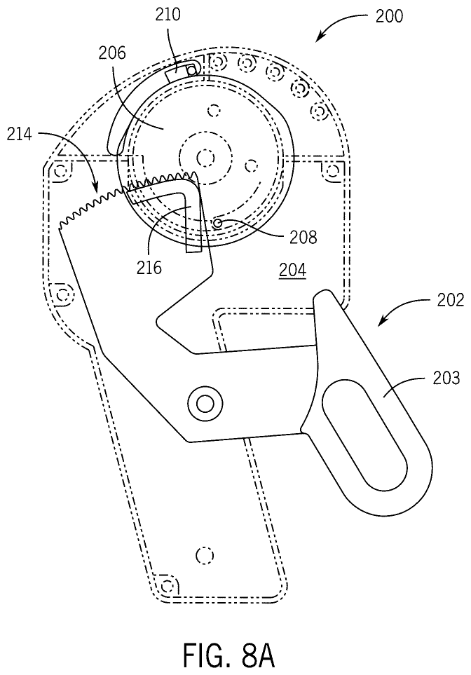

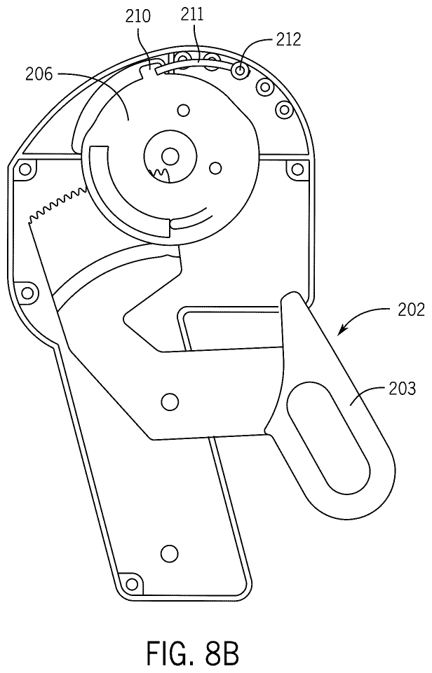

One or more of the aforementioned components, assemblies, subassemblies, or mechanisms may be modified, reconfigured, removed, replaced, and/or combined with additional embodiments of a reloadable and/or multi-fire delivery device disclosed herein. For example, FIG. 8A provides a transparent view of a handle assembly 200 of another reloadable delivery device (minus the shaft assembly, which is included with, and extends longitudinally from, the handle, as in FIGS. 5A and 5B) configured to couple with a removable cartridge. The handle assembly 200 may include an actuator 202 defining a manually engageable portion 203 (e.g., trigger) and an internal portion within a handle case, positioned between a left side of a handle case 204 and a second, complementary right side of the handle case (both shown in transparency for ease of illustration and to show the internal components of the handle assembly). As described in greater detail below and further shown in FIG. 8B, the handle assembly 200 may also include a needle spool member 206 having a raised or protruding portion, e.g., post portion 208 (shown e.g., on FIG. 8A), and a tab 210. The tab 210 may be attached to a spring 211 connected at its opposite end to a spring attachment post 212. The actuator 202 may define or otherwise include internal gear teeth 214 and a raised rail portion 216. Although not depicted again in FIG. 8A or 8B, the handle assembly 200 may also include one or more of the components described above in connection with handle assembly 100, such as the shaft assembly 104 included or coupled therewith.

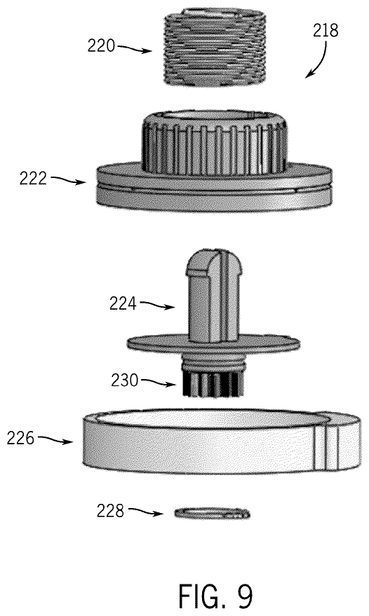

FIG. 9 provides an exploded view of the components included in a removable cartridge configured to couple with handle assembly 200. As shown, the cartridge may include a suture spool assembly 218 featuring a spring 220, a suture spool member 222, a tension spool member 224, a housing 226, and a retaining ring 228. The tension spool member 224 may include or define a gearing portion 230 configured to engage the gear teeth 214 of the actuator 202. The tension spool member 224 and suture spool member 222 may be coupled via the spring 220 such that upon activation of the actuator 202, the suture spool member 222 and tension spool member 224 may rotate together if there is no tension on the suture. Once the suture is tensioned after its deployment in the targeted prostatic tissue, the suture spool member 224 may stop rotating. The tension spool member 224, by contrast, may continue rotating until the maximum force is reached by the spring 220.

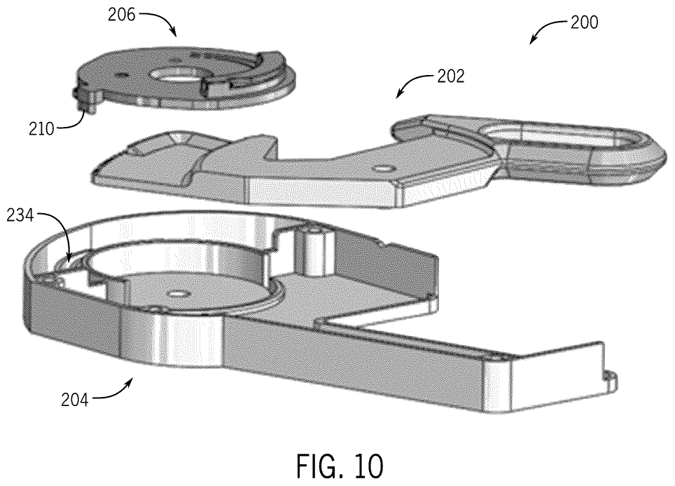

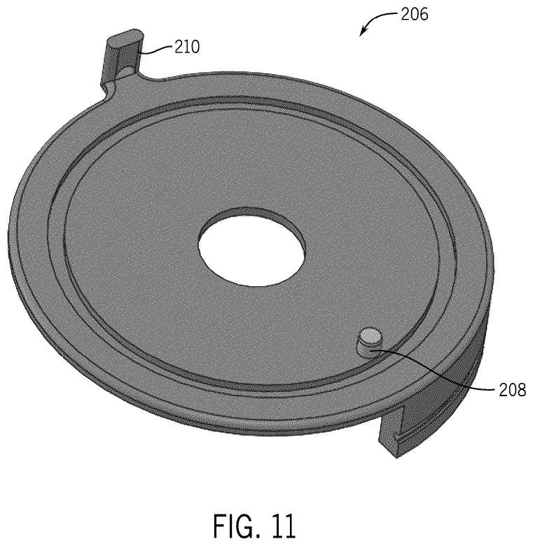

An exploded view of the handle assembly 200 is provided in FIG. 10, showing the needle spool member 206, actuator 202, and bottom or left side of the handle case 204. FIG. 11 provides an enlarged view of the underside of the needle spool member 206 (relative to its depiction in FIG. 10), showing the post portion 208 and the tab 210. In operation, the post portion 208 is contacted and moved by the rail portion 216 of the actuator 202 as the actuator 202 is engaged (e.g., squeezed) by a user, causing a delivery needle (e.g., delivery needle 162) to extend beyond the distal end of a shaft assembly (e.g., shaft assembly 104) and into the targeted prostatic lobe. Accordingly, the needle spool member 206 of this embodiment may not be driven by a gearing mechanism, thus lacking the gear teeth included on needle spool member 110, for example. A tension spring 211 may be attached to the tab 210, biasing the needle spool member 206 toward a first position, within the handle assembly 200.

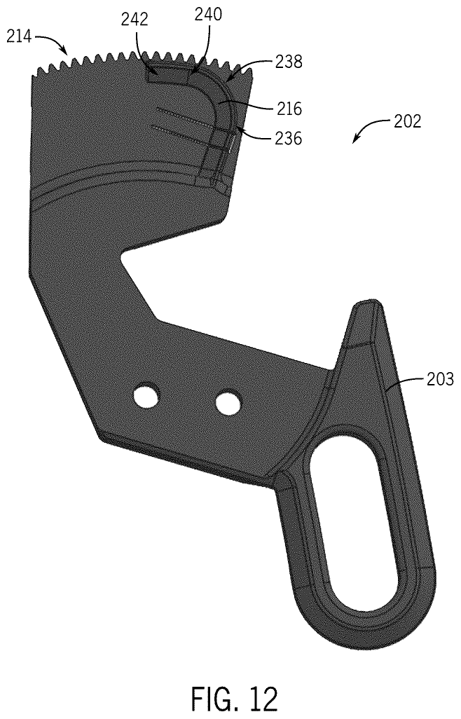

FIG. 12 shows the actuator 202, including the actuation gear teeth 214 and rail portion 216. The gear teeth 214 may engage complementary gear teeth defined by one or more components of the spool assembly 218, such as gearing portion 230. Activation of the spool assembly 218, including the resulting distal advancement of a CT/suture assembly down the shaft assembly and into a targeted prostatic lobe, may be achieved in a manner that is substantially the same or similar to that described above in connection with suture actuator 114 and suture spool assembly 156. For instance, pulling or squeezing the actuator 202 may cause rotation of one or more components of spool assembly 218, causing the CT/suture assembly to unwind and extend through the needle.

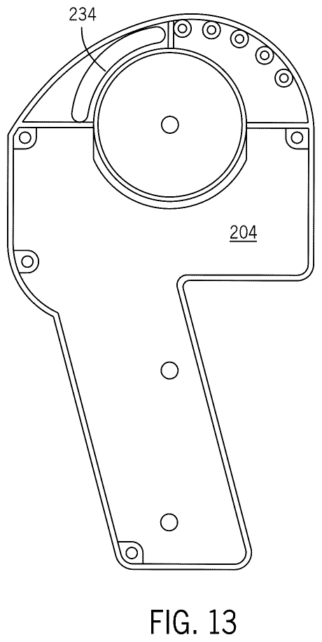

As shown in FIG. 13, the bottom or left side of the handle case 204 may also include a track 234. The track 234 may be configured to accommodate and guide movement of the tab 210, such that during operation, the tab 210 may ride on or in the track 234 in a controlled fashion.

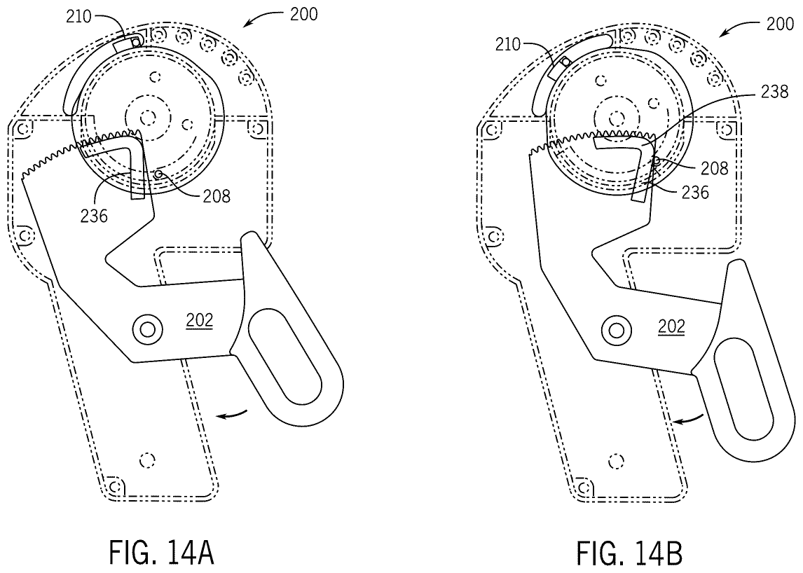

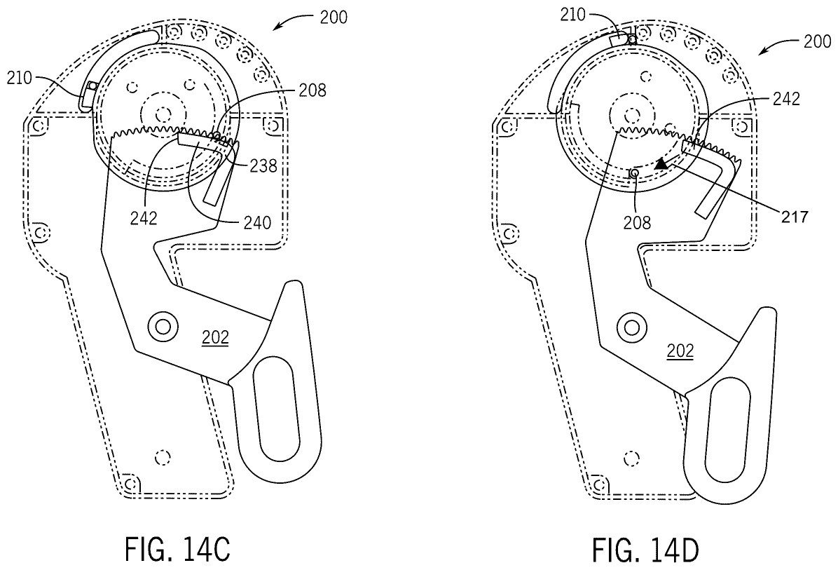

FIGS. 14A-14D illustrate snapshots of the handle assembly 200 during activation of the delivery device to advance a needle and a CT/suture assembly through a shaft assembly, into the targeted prostatic tissue. FIG. 14A depicts the handle assembly 200 in a resting configuration, which may be considered a first position in which the actuator 202 has not been squeezed and the rail portion 216 remains separated from the post portion 208. Upon squeezing the actuator 202 toward the body of the handle assembly 200 (in the direction of the arrow), a first segment or portion 236 of the rail portion 216 may contact and push the post portion 208 (counterclockwise to the right, from the vantage point shown in FIG. 14B), causing the needle spool member 206 to rotate, such that the tab 210 slides along the track 234 and the spring attached at one end to the tab 210 and at the opposite end to the spring post 212 is transitioned from an unloaded to a loaded configuration, in which the spring is stretched in this particular example. Rotation of the needle spool member 206 causes the needle coupled therewith to unwind and advance distally through the shaft assembly, eventually into and through the targeted tissue until its distal tip pierces the prostatic capsule. Rotation of the needle spool member 206 may continue with additional squeezing of the actuator 202 until the post portion 208 reaches an arcuate corner portion 238 of the rail portion 216. From there, continued squeezing of the actuator 202 may cause the post portion 208 to slide around the corner portion 238, onto a second portion 240 of the rail portion 216 (FIG. 14C). During this time, the gear teeth 214 of the actuator 202 may continue rotating the complementary gearing portion 230 of the tension spool member 224 to advance the CT/suture through the now fully extended needle. The post portion 208 may slide along the second portion 240 of the rail portion 216 until reaching the end 242 of the rail portion 216, after which the post portion 208 may disengage (i.e., slide off) the rail portion 216 and return (see arrow 217 in FIG. 14D) to its starting position in response to the biasing force applied by the tension spring, thereby rotating the needle spool member 206 back to its initial resting configuration and retracting the needle back into the shaft assembly of the delivery device, thereby unsheathing the implanted CT/suture. The actuator 202 may then be gently released to pull the CT against the outer surface of the prostate capsule and tension the suture within the targeted lobe. The UE may then be attached to the suture at the urethral side of the lobe, and the suture cut just proximal to the UE, as shown and described herein.

Accordingly, the embodiment shown in FIGS. 8A-14D may be configured to deliver and tension a CT/suture to a target site via one manual actuation, e.g., trigger pull, which unspools a needle included in the handle assembly and a CT/suture included in the cartridge. Like the embodiment shown in FIGS. 5A-7F, the device components represented in FIGS. 8A-14D enable the delivery of multiple implants via repeated cartridge replacement in a treatment procedure without removing the elongate shaft assembly from the patient.

As shown in FIGS. 8A-14D, delivery devices and systems disclosed herein can deploy multiple implants to a prostate gland of a human subject without removing the elongate shaft assembly of the delivery device from the subject. Embodiments of such devices/systems may include a handle assembly and a removable cartridge. The removable cartridge may be configured to contain all or a portion of at least one distal anchor component (CT) and a connector portion (e.g., suture) attached thereto. The distal anchor component may be configured for attachment to an outer surface of the prostatic capsule, and the suture may be configured to be placed within, and extend through, the prostate tissue. The handle assembly may include or be attached to at least one actuator, a needle configured to receive and house the CT/suture and pierce through the prostate gland, and an elongate delivery shaft assembly configured to be advanced longitudinally through the urethra of the subject while accommodating advancement of the needle therethrough. The removable cartridge can be detached, decoupled, disengaged, or otherwise removed or separated from the handle assembly after insertion of the shaft assembly within the urethra and without removing the shaft assembly from the urethra. One or more of the actuator(s) of the delivery device can include a gearing portion or feature, e.g., gear teeth, and an engagement feature or protrusion, e.g., a raised rail portion. Embodiments of the delivery device can include at least one spooling mechanism, which may include multiple spools or spool members. For example, the spooling mechanism may include one or more spool members in the handle assembly and one or more spool members in the removable cartridge. The spool member(s) included in the handle assembly may comprise a needle spool member, i.e., a spool member configured to couple with the needle of the delivery device. Embodiments of the needle spool member may lack a gearing feature or portion, e.g., gear teeth. Embodiments of the needle spool member may also include a protrusion or post portion, along with a tab portion or member. The tab member can be attached to a spring member, and the post portion can be configured to contact and be moved by the raised rail portion of the actuator in response to manual engagement (e.g., squeezing or moving) of the actuator, which can cause the needle to unwind from the needle spool member and advance distally through the elongate shaft assembly. In some embodiments, the post portion can slide along the raised rail portion as the actuator continues to move until reaching an end of the raised rail portion, at which point the post portion can disengage the raised rail portion and retract the needle in the proximal direction. In some embodiments, movement or engagement of the actuator can advance the CT/suture distally through the needle.

One or more of the aforementioned components, assemblies, subassemblies, or mechanisms may be modified, reconfigured, removed, replaced, and/or combined with additional embodiments of a reloadable and/or multi-fire delivery device disclosed herein. For example, FIG. 15A provides an exploded view of the components included in a removable cartridge loaded with both a CT/suture assembly and a delivery needle (e.g., needle 162). The removable cartridge 250 may be coupled with a handle assembly before or during a procedure. The handle assembly may share one or more components, or be otherwise similar, to handle assembly 100 or 200, featuring or attached to two handle halves or case components, an elongate shaft assembly, a manually engageable actuator, along with the subassemblies necessary to place the UE and cut the suture.

As shown, components of the cartridge 250 may include an elongate needle 251, which may be biased to curve at its distal end and configured to house and deliver a CT/suture assembly after piercing and extending through the targeted prostatic tissue. The cartridge 250 may also include a cartridge base 252 configured to contain components of the cartridge, a cartridge cover 254, and a knob 256 configured to lock the cartridge assembly into a compatible handle. The cartridge 250 may further include a needle spool member 258, a needle spool cover 260 or housing, a suture spool member 262, a first gear 264, and a second gear 266. The knob 256 may be connected to the first gear 264.

During cartridge installation, manual rotation of the knob 256 may lock the cartridge 250 to its handle assembly and rotate the first gear 264, which in turn may rotate the needle spool member 258 via rotation of the second gear 266 connected thereto, feeding the needle 251 through the shaft assembly 268 to the prostatic urethra, where the distal end 273 of the shaft assembly, which may define an exit port 275 for passage of the needle, is positioned. The needle spool member 258 may be connected to the needle 251, providing a track for the needle 251 to wrap around one or more times. The needle is long enough to feed through the shaft assembly 268 (after extending through a needle opening defined by the shaft assembly holder 270) and the targeted prostatic lobe.

The needle spool member 258 may include or define a flexure on its underside (relative to the illustrated view) configured to latch, grab onto, or otherwise capture the suture after the CT has been unsheathed at the outer surface of the prostatic capsule. This mechanism may allow the suture spool member 262 to rotate at the same speed as the needle spool member 258 and apply gentle tensioning to the CT/suture after its implantation.

The needle spool cover 260 houses the needle spool member 258 and keeps the needle 251 constrained within a needle track 259 defined by the needle spool member 258. As further shown in FIGS. 15B and 15C, a pass-through hole 271 included in the shaft assembly holder 270 accommodates passage of the needle 251, enabling it to travel from the needle spool member 258 into the proximal end of the shaft assembly 268. The needle spool cover 260 may also include or define a flexure 261 configured to engage with a complementary protrusion or catch portion 263 included in the suture spool member 262 (see FIGS. 15D-E) to catch the suture spool member 262 during needle retraction to unsheathe the CT, after which the flexure 261 may be released by a protrusion 265 on the needle spool member 258. This allows for the suture spool member 262 to be caught by the needle spool member 258 in the manner described above.

The CT/suture assembly may connect to and be housed within the needle spool 258. As shown in FIG. 15F, there may also be two cutouts 267a, 267b or slots at the bottom of the suture spool member 262, one cutout configured to allow movement of the needle spool member 258 during CT unsheathing, and the second cutout configured to allow for the connection between the needle spool member 258 and the suture spool member 262 during gentle tensioning of the CT/suture. A flexure 269 may be included in the needle spool member 258, the flexure 269 configured to engage a wall portion 271 defined between the two cutouts 267a,b.

The second gear 266 may interact with both the knob 256 (via first gear 264) and the actuator (e.g., actuator 202, or something similar) of the handle assembly (e.g., handle assembly 100 or 200, or something similar). When the actuator is pulled or squeezed, the second gear 266 may rotate to deploy the needle through the prostatic tissue and deploy the implant. Release of the actuator may then retract the needle 251 and activate suture tensioning.

The cartridge exchange process may be completed without removing the shaft assembly 268 from the patient, such that the delivery device as a whole may remain stationary through multiple exchanges until a given procedure has been completed. This may reduce the number of exchanges necessary, improve patient comfort, reduce or prevent unwanted tissue manipulation, reduce the procedure time, and allow for visualization to be maintained at all points during the procedure.

As shown in FIGS. 15A-15F, embodiments of a delivery device are configured to deploy multiple implants to a prostate gland of a patient without removing an elongate delivery shaft of the delivery device from the patient. Embodiments of the delivery device can include a handle assembly and a removable cartridge (e.g., cartridge 250). The removable cartridge can be configured to contain at least one distal anchor component and suture attached thereto, along with a needle configured to pierce through the prostate gland (e.g., needle 251). The handle assembly can include an actuator, and the elongate delivery shaft can be advanced longitudinally through the urethra of a patient and accommodate advancement of the needle through the delivery shaft. The removable cartridge containing or coupled with the needle (or needle assembly) can be loaded into the side of the handle assembly in some examples. A locking component, such as a knob, switch, turnkey or other rotatable, slidable, or movable component, may be included, for example on or as part of the cartridge. The locking component (e.g., knob 256) can be configured to lock the cartridge assembly into the compatible handle assembly, thereby loading the resulting device with the CT/suture assembly and needle included in the cartridge. The cartridge can further include a needle spool member (e.g., needle spool member 258), i.e., a spool member configured to couple with the needle of the device. Also included in the cartridge is a suture spool member (e.g., suture spool member 262), i.e., a spool member configured to couple with the suture of the device. One or more gearing features, such as a first gear member (e.g., first gear 264) and a second gear member (e.g., second gear member 266), can also be included in the cartridge and may be rotatably coupled. The locking component can be connected to at least one of the gear members. For example, the locking component may be connected to the first gear member, such that rotation of the locking component may rotate the first gear, which in turn may rotate the needle spool member via rotation of the second gear member. This gearing interaction can feed the needle of the cartridge through the shaft assembly en route to the prostatic urethra. Accordingly, the first gear member can be configured to engage with the locking component on the cartridge during cartridge installation, such that locking the cartridge to the handle assembly via rotation of the locking component drives rotation of the needle spool member and advancement of the needle into and through the elongate delivery shaft.

In some examples, the ability to leave the device in the subject throughout the procedure may also eliminate the need for a sheath that would otherwise be inserted through the urethra to provide a passageway for successive removal/reinsertion of a shaft assembly. This may reduce the effective diameter of the elongate shaft assembly 268 necessary to perform the procedure, further improving patient comfort. Additionally, because the needle 251 is included with the cartridge 250 in this embodiment, the handle assembly may be preserved in the event of damage to the needle 251. If the needle 251 is damaged, the cartridge 250 can simply be replaced, again without removing the shaft assembly from the subject.

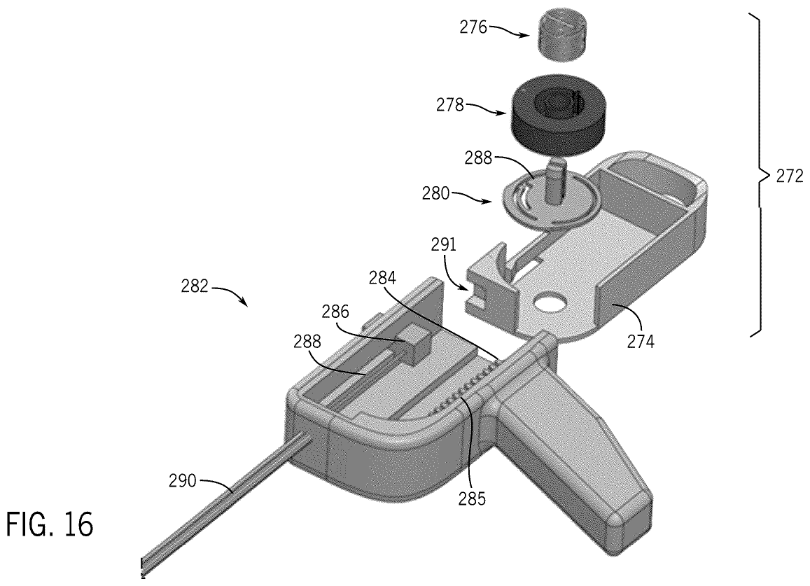

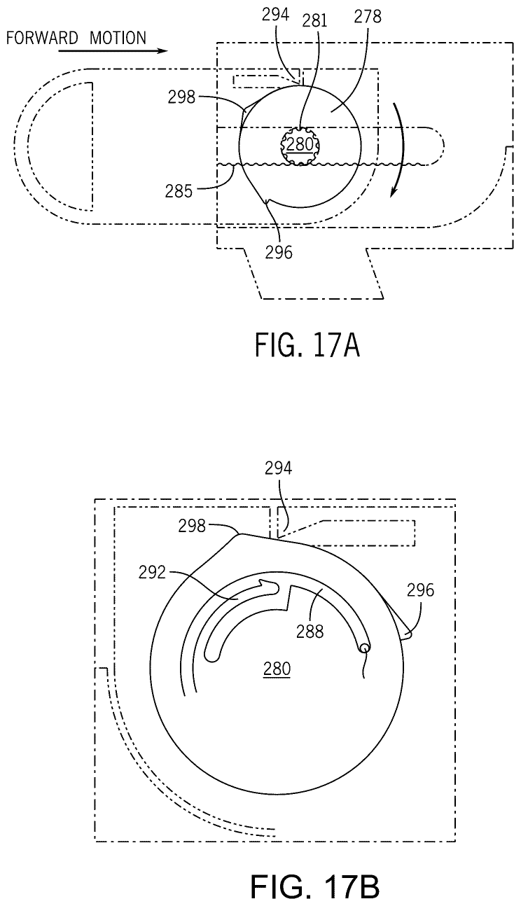

In some embodiments, a removable cartridge may be loaded from the rear of the handle assembly, instead of the side. In accordance with such embodiments, forward motion of the cartridge into the handle assembly when coupling the two components may simultaneously unspool the needle and/or the CT/suture into the handle pursuant to the implant deployment process. FIGS. 16, 17A and 17B illustrate an example of such an embodiment, showing the components of a reloadable or removable cartridge 272, including a cartridge housing or base 274 configured to contain a tension spring 276, a suture spool member 278, and a needle spool member 280 having a geared portion 281.

The associated handle assembly 282 may include or be attached to, among other things, a handle member 284 with a gear rack 285, a slider member 286, a guide tube 288, and an elongate shaft assembly 290. A slot 291 may be included in the cartridge base 274, configured to mate with the slider member 286 and align the cartridge components with the corresponding components of the handle assembly 282 after coupling. A snapshot of the associated cartridge loading process is shown in FIG. 17A, and a snapshot of the spool firing mechanism is shown in FIG. 17B.

Via its interaction with the geared portion 281 of the needle spool member 280, the gear rack 285 of the handle may translate the linear forward motion of the rear-loading cartridge 272 into rotary motion of the needle spool member 280, thereby unspooling the needle and CT from the cartridge 272. The slider member 286 may mate with the cartridge 272 during the loading process to funnel the needle and CT directly into the guide tube 288, which is configured to bridge the gap between the proximal end of the shaft assembly 290 and the cartridge 272 during the cartridge loading process. The needle may thus be fed through the guide tube 288 and into the shaft assembly 290 immediately upon coupling the cartridge with the handle. A geared actuator (e.g., actuator 202, or something similar), may mechanically drive the suture spool member 278 and needle spool member 280 to fire the needle and CT, subsequently retract the needle, unsheathe the CT, and tension the suture, all in one complete actuation or trigger cycle.

The tension spring 276 may be configured to allow tensioning of the suture at a specific force when the cartridge spools are spooled back in the device. The suture spool member 278 may connect to the CT/suture assembly and mate with the needle spool member 280. The suture spool member 278 may include or define a protrusion or post portion 287 configured to move within a curved slot 288 defined by the needle spool member 280, tying the motion of the needle spool member 280 to the suture spool member 278 as the post portion 287 moves from its position on the back wall of the curved slot 288 via gear-driven rotation of the needle spool member 280. The needle spool member 280 may also include a flexure 292 configured to engage or catch post portion 287 as the needle spool member is retracted, thereby tying the motion of the suture spool member 278 with the needle spool member 280, which allows for a moderated gentle tensioning of the suture. Once the CT has anchored on the prostatic capsule and the suture is tensioned, the suture may be left behind and post portion 287 may separate from flexure 292. A cartridge base flexure 294 may also be included, configured to contact a protruded portion 296 of the suture spool member 278, thus providing a stop for the suture spool member 278 during suture retraction/tensioning, while allowing the needle to continue retracting via rotation of the needle spool member 280. The cartridge base flexure 294 may be released after contacting a needle spool protrusion or bump 298 that, upon continued rotation of the needle spool member 280, temporarily urges the flexure 298 radially outward, away from the needle spool 280.

As shown in FIGS. 16-17B, embodiments of a delivery device are configured to deploy multiple implants to a prostate gland of a patient without removing an elongate delivery shaft of the delivery device from the patient. Embodiments of the delivery device can include a handle assembly and a removable cartridge (e.g., cartridge 272), the removable cartridge configured to be loaded into the back or rear of the handle assembly. The removable cartridge can include a spooling mechanism that includes one or more spool members, including a suture spool member (e.g., suture spool member 278) and a needle spool member (e.g., needle spool member 280). One or both of the spool members may include a gear feature, surface or portion (e.g., geared portion 281) configured to interact with a complementary gear feature, surface or portion of the handle assembly (e.g., gear rack 285). The gear portion of the handle assembly may be linear, straight, or flat, resembling a geared track, and the gear portion of the spool member(s) can be arranged in a circular manner, e.g., wrapped circumferentially around the spool member(s). Insertion of the cartridge into the handle assembly, for example via sliding of the cartridge into a rear portion of the handle assembly, can cause the gear feature of the spool member(s) to interact with the complementary gear feature of the handle, such that lateral or linear insertion of the cartridge into the handle assembly causes rotation of the spool member(s) and unwinding of the component(s) coupled thereto, such as a needle or CT/suture. Rotation of the spool members, e.g., needle spool member and suture spool member, can be tied together, for example via one or more intermediary components or features of the device (e.g., post portion 287 and/or flexure 292). One or manually engageable actuators (e.g., actuator 202) operatively coupled with additional subassemblies of the device can drive the complete deployment of each implant assembly.

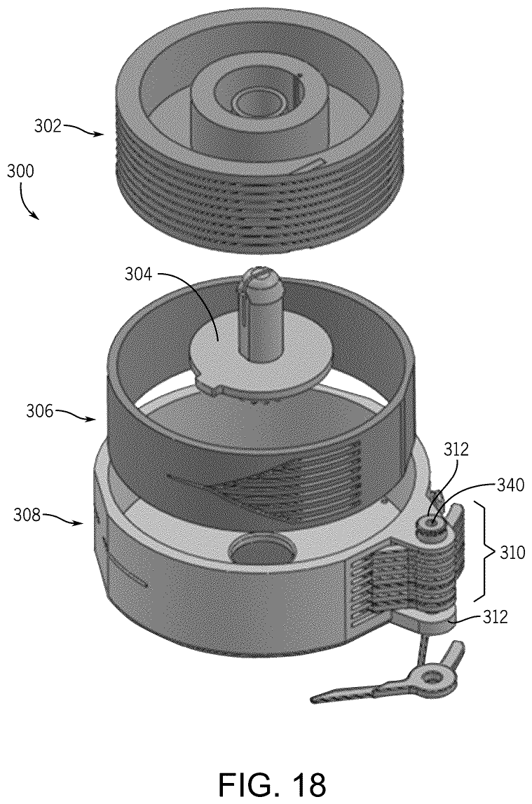

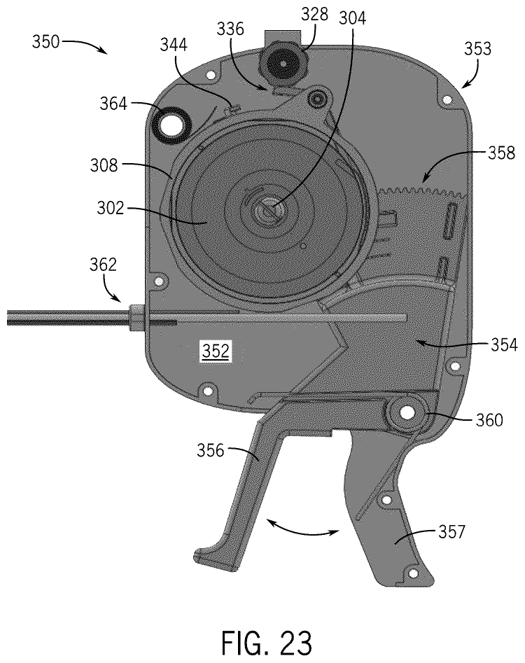

In some embodiments, a delivery device may be configured to simultaneously hold and serially deploy multiple implants without removing an elongate shaft assembly of the device from a patient during a procedure. Such devices may be referred to as “multi-fire” devices, which may lack a reloadable or removable cartridge. Examples of such a device may include a manifold component coupled or integrated with an internal spooling mechanism. As shown in FIG. 18, for instance, a multi-fire delivery device may include a manifold spool assembly 300 configured to be included, attached to, or coupled with a handle assembly. The manifold spool assembly 300 may include a suture spool member 302, a suture spool gear 304, a cylindrical manifold member 306, a needle spool member 308, and a plurality of pivotable members or flexure plugs 310 (resembling music box fingers) coupled with the needle spool member 308 via a holder portion 312. The manifold spool assembly 300 may be configured to be seated, inserted, or otherwise coupled with a handle assembly that includes an actuator, an elongate shaft assembly, a needle retraction spring, and/or a cam indexer, as further shown in FIGS. 23-25. In response to manual actuation of the delivery device, these components may interact to funnel pre-loaded prostatic implants into the lumen of a delivery needle, one-by-one, and advance them to the distal tip of the needle from which they are then ejected or deployed into a targeted prostatic lobe.

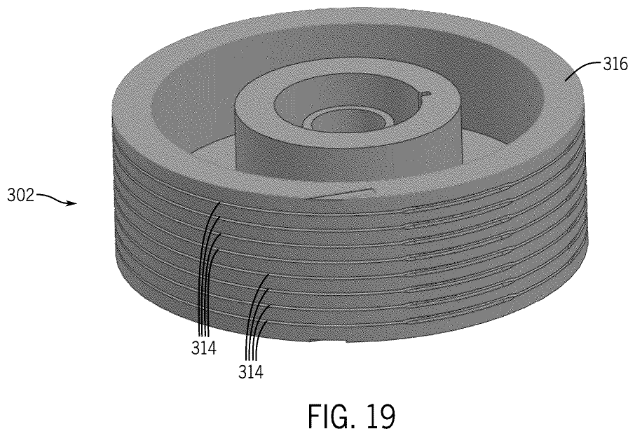

As shown in FIG. 19, the suture spool member 302 may include a plurality of CT/suture tracks 314 around which the CT/suture assemblies may be simultaneously wound within the device. Each track 314 may hold or accommodate one CT/suture assembly, such that the CT/suture capacity of the manifold spool assembly 300, and multi-fire device as a whole, matches the number of CT/suture tracks 314. The CT/suture tracks 314 may be parallel or substantially parallel, wrapping circumferentially around the cylindrical body 316 of the suture spool member 302. Embodiments of the suture spool member 302 may include various numbers of tracks, including one, two, three, four, five, six, seven, eight, nine, ten tracks, or more, depending on the number of implants necessary for a given procedure. The manifold spool assembly 300 and delivery device as a whole may thus be selected based in part on its CT/suture capacity defined by the number of CT/suture tracks 314. The delivery device may be loaded with the number of implants necessary for a given procedure, up to the maximum CT/suture count of the device. If eight implants are necessary to complete a procedure, for example, a suture spool member 302 featuring at least eight CT/suture tracks 314 could be used, each track having a CT/suture assembly wrapped around the body of suture spool member 302.

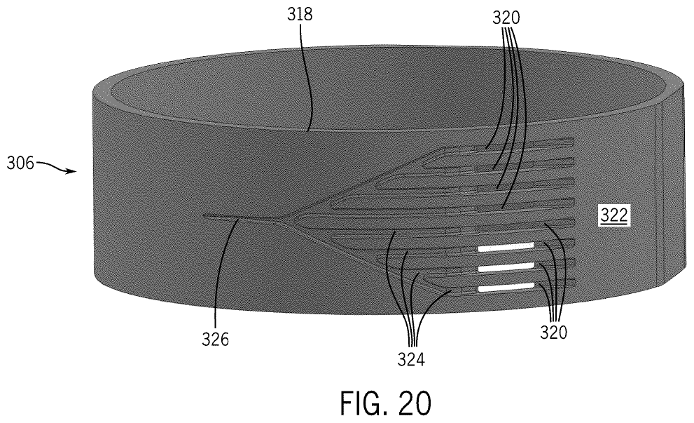

The manifold member 306 shown in FIG. 20 includes a manifold 318 comprising a plurality of openings 320 each configured to accommodate the passage of one CT/suture assembly therethrough from a corresponding track 314 of the suture spool member 302. The openings 320 transition into a series of channels 324 defined by the outer surface 322 of the manifold member 306. The channels 324 ultimately converge into a single needle delivery track 326 that may feed directly into the lumen of a delivery needle (e.g., needle 162) coupled with the needle spool member 308. The needle spool member 308 may house both the suture spool member 302 and the manifold member 306. In operation, rotation of the needle spool member 308 may operate to deploy the needle.

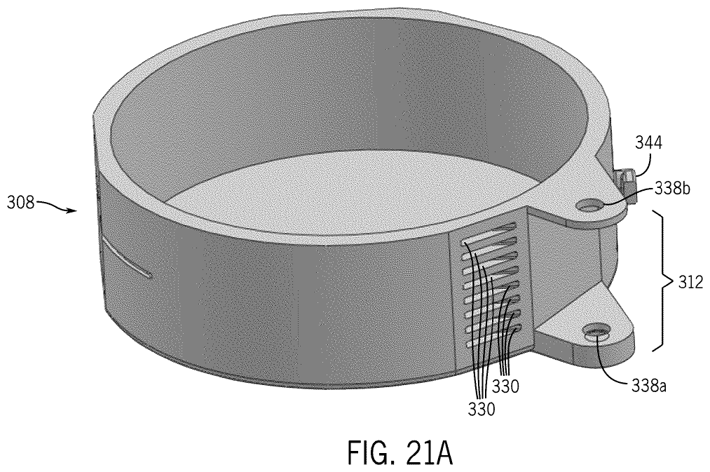

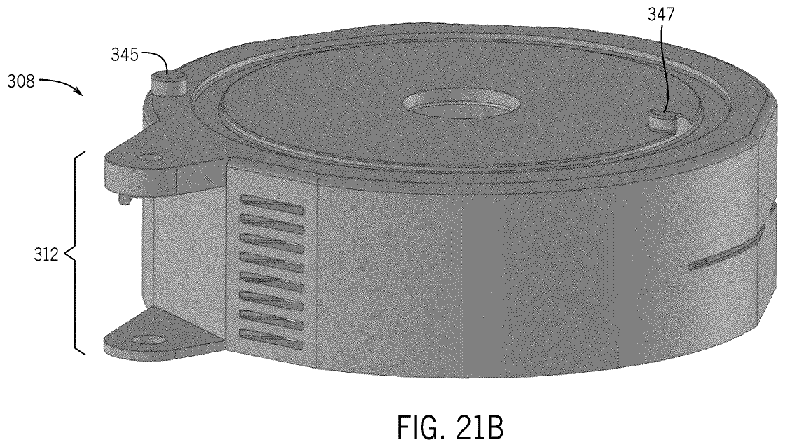

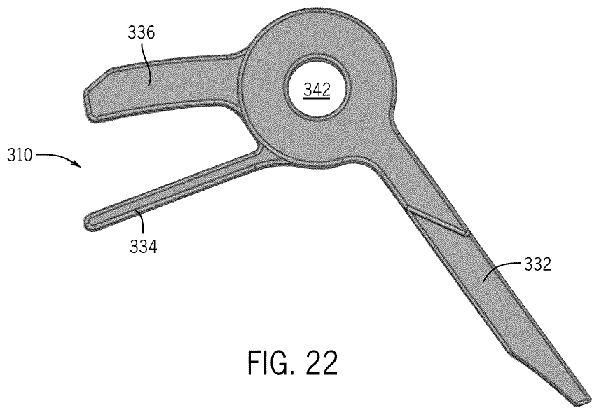

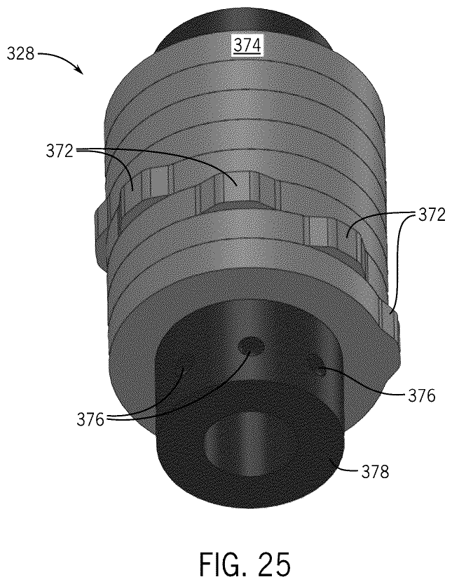

To ensure that only one CT/suture assembly is extended into the needle at a time, the openings 320 of the manifold may be selectively uncovered by one or more other components of the device, which may be movable or pivotable, such as the flexure plugs 310, in response to rotation of a cam indexer 328 (see FIGS. 23 and 25) included in the handle assembly of the delivery device. As further shown in FIGS. 21A, 21B and 22, the needle spool member 308 may include a plurality of needle spool openings 330, each opening configured to accommodate the insertion of a single protrusion, arm, extension or component of a flexure plug 310, such as finger portion 332. The flexure plug 310 may include a number of additional features, components, or portions, such as a flexure portion 334 and an arm portion 336. The holder portion 312 for the flexure plugs 310 may include two apertures 338a, 338b through which a coupling pin 340 may be inserted (see FIG. 18). Each flexure plug 310 may define a corresponding aperture 342 also configured to accommodate insertion of the coupling pin 340. When coupled to the needle spool member 308 via the holder portion 312, the flexure plugs 310 may be configured to block the manifold openings 320 and prevent the CTs (and sutures attached thereto) from exiting the suture spool member 302 in a controlled fashion. The flexure portion 334 may bias each flexure plug 310 to the closed position, covering its respective manifold opening 320, and the arm portion 336 may interact with the cam indexer 328 to open and close each respective needle spool opening 330 upon rotation of the cam indexer 328. The needle spool member 308 may also include a hook member 344 configured for attachment to a needle retraction feature, e.g., spring, in the handle. The needle spool member 308 may further include a needle post portion 345 and a boss portion 347, as shown in the view provided by FIG. 21B.

FIG. 23 shows a portion of a delivery device 350 containing the manifold spool assembly 300 seated within a bottom or right-hand side of the handle case 352 of a handle assembly 353, which also includes a top or left-hand side of the handle case not shown in the illustration solely to reveal the inner components of the delivery device 350. The delivery device 350 includes an actuator 354, e.g., a trigger or lever, defining a manually engageable portion 356 protruding from the handle assembly 353, spaced from a manually engageable portion 357 of the handle case 352. The actuator 354 includes internally positioned actuator gear teeth 358, an actuator spring 360 coupled with the actuator 354, a shaft assembly 362, the cam indexer 328, and a needle retraction spring 364. The suture spool member 302, needle spool member 308, suture spool gear 304, and arm portion 336 of a flexure plug 310 are also shown. Additional embodiments may utilize alternatives to the flexure plugs, non-limiting examples of which may include rotational fobs or other rotatable, slidable, or otherwise movable components, which may pivot about an axis or a hinged feature.

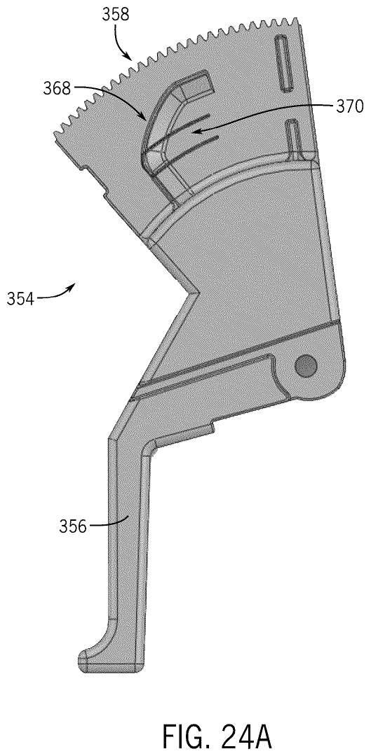

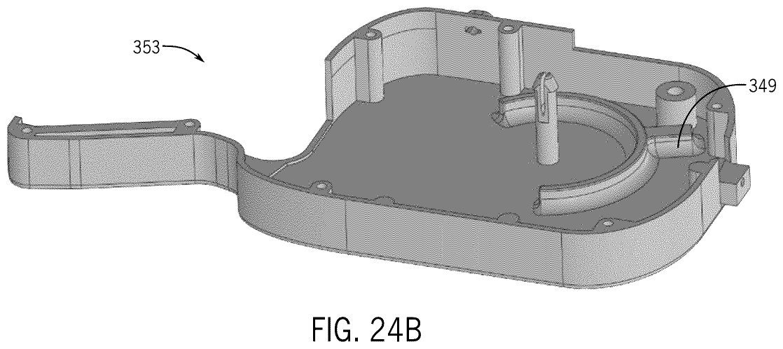

FIG. 24A provides an illustration of the actuator 354 or trigger, including the manually engageable portion 356 and the gear teeth 358. As further shown, the actuator 354 may include or define a protruding wall portion 368 and a flexure 370, both of which may be configured to interact with the boss portion 347 to rotate the needle spool member 308 and deploy the needle. The needle post portion 345 may interact with a case wall portion 349 included in the handle case 352 (see FIG. 24B) to prevent further back-rotation of the needle spool member 308 relative to the starting position of the needle spool member 308. In operation, the gear teeth 358 of the actuator 354 may interact with the suture spool gear 304, such that squeezing the manually engageable portion 356 of the actuator 354 toward the manually engagement portion 357 of the handle case causes rotation of the suture spool gear 304 and suture spool member 302. The wall portion 368 of the actuator 354 simultaneously rotates the needle spool member 308 by interacting with the protruding boss portion 347 on the needle spool member 308. The needle retraction spring 364 may be attached to a spring post on the handle case 352 and the hook member 344 of the needle spool member 308. Once the needle spool member 308 fully rotates, thereby advancing the needle distally through the shaft assembly 362, the needle spool post may disengage (e.g., fall or slide off) the wall portion 368 and be reset to its initial starting position. The flexure 370 is configured to deflect the needle spool post such that the post bypasses the actuator 354 when it resets to its starting position, i.e., when the manually engageable portion 356 moves back away from the manually engageable portion 357 of the handle case in response to the needle retraction spring 358.

FIG. 25 provides an illustration of the cam indexer 328, including the plurality of protruding nose portions or lobes 372 protruding radially outward from an outer body portion 374, as well as a plurality of detents 376 extending radially inward from the outer surface of an inner body portion 378. As shown, the lobes 372 may be circumferentially spaced around the outer body portion 374. The number of lobes 372 may match the number of CT/suture assemblies to be delivered. The detents 376 may be configured to stop the cam indexer 328 at a position where a lobe 372 acts on an arm portion 336 of a flexure plug 310 by pressing the arm portion 336 radially inward, toward the body of the needle spool member 308, causing the associated finger portion 332 to extend away from the body of the spool member 308, out of its corresponding needle spool opening 330. Uncovering the needle spool opening 330 may allow a CT/suture to be fed through its respective manifold opening 320, into its respective channel 324, down the needle delivery track 326, and into the lumen of a needle.

Unspooling and ultimately delivering each CT/suture may be driven via manual engagement (e.g., squeezing) of the manually engageable portion 356 of the actuator 354, which causes rotation of the suture spool member 302 via the interaction between the suture spool gear 304 and complementary gear teeth 358. The suture spool member 302 may be rotated first to pass the CT/suture through the manifold 318 and into the needle. Then, the actuator 354 may engage the needle spool post and rotate the needle spool member 308. As the needle spool member 308 rotates, the cam indexer 328 loses contact with the arm portion 336 of the flexure plug 310, allowing the finger portion 332 to close on the suture, but remain biased open. As the needle is retracted, the cam indexer 328 will act on the arm portion 336 again, re-opening the finger portion 332. Once implantation of the CT/suture is complete and a UE is coupled to the suture thereby completing an anchor assembly, the cam indexer 328 rotates until the next adjacent lobe 372 contacts and depresses the next adjacent arm portion 336 of the next flexure plug 310, thereby opening the associated finger portion 332 and uncovering the needle spool opening to allow passage of the next loaded CT/suture through the manifold 318 and into the lumen of the needle, now reset to its original position. The delivery process may be repeated until all CT/sutures wound around the suture spool member 302 are deployed.

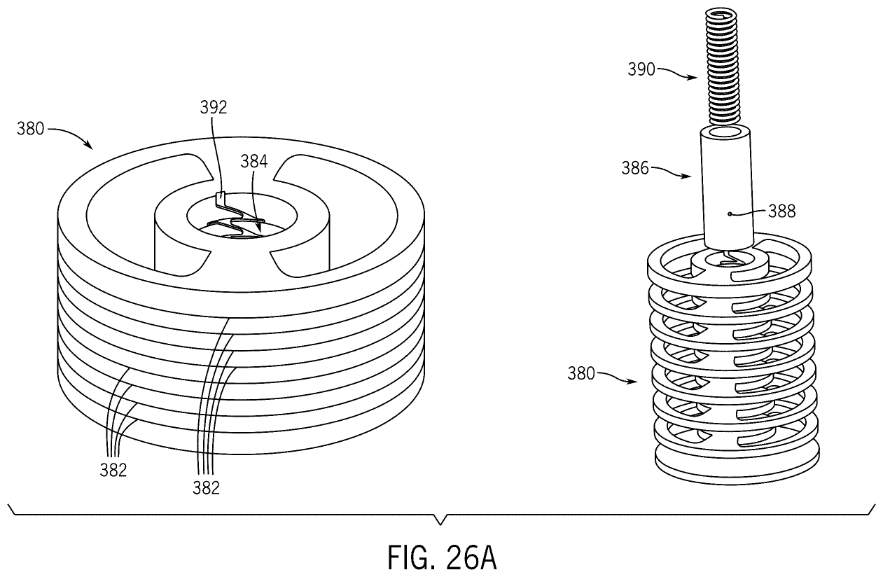

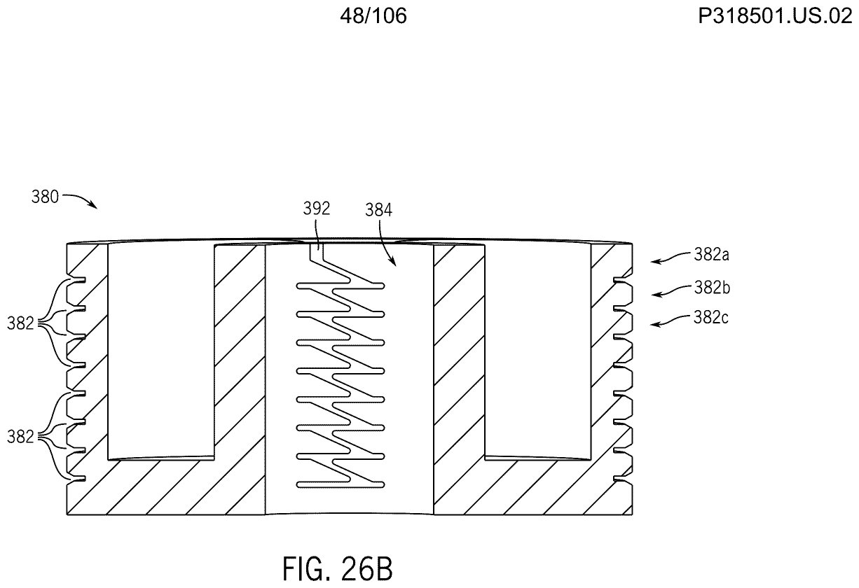

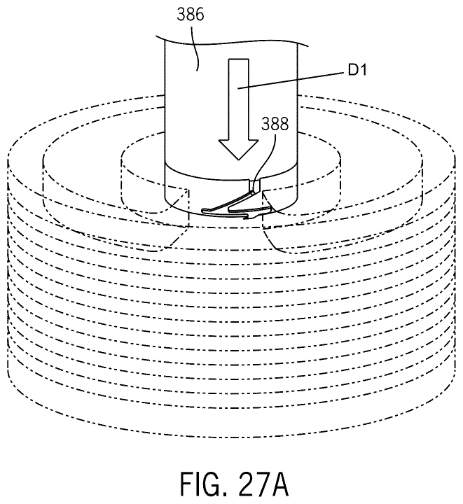

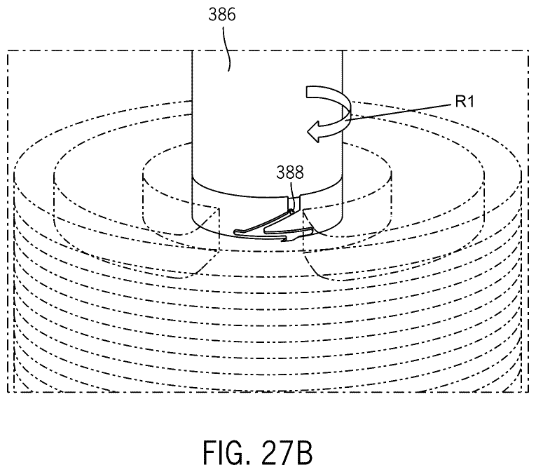

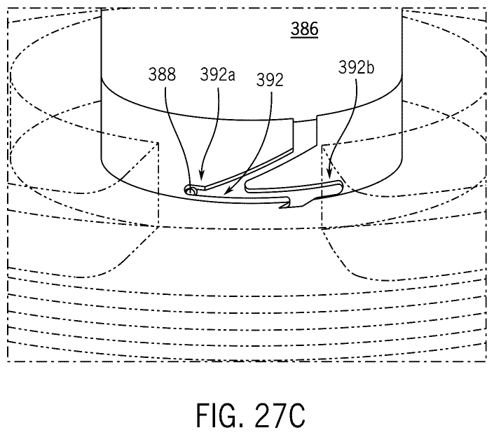

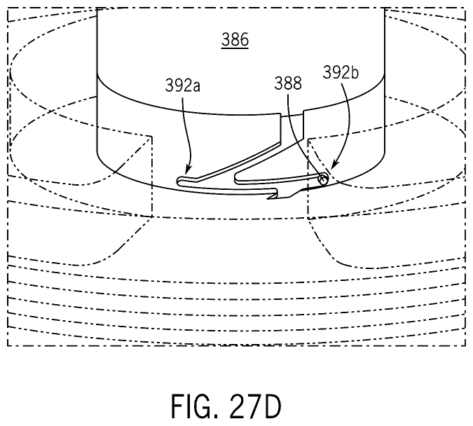

Additional multi-fire devices may include a manifold to funnel each CT/suture into a needle, but instead of all implants rotating in unison around the same spool member, a spool member featuring individually rotatable spools or spool components may be used. For example, FIGS. 26A and 26B show a spool member 380 that includes eight independently rotatable spools 382 (or spool components, portions or members) and a central cam pathway 384. As shown on the exploded (right) view of FIG. 26A, a spring-loaded rod 386 including a laterally protruding bore pin member 388 and coupled with a spring 390 may be configured to act on the spool 380 and may be connected to a gear that rotates the suture spool. As the gear rotates, the spring 390 may push down on the rod 386 (see arrow D1 in FIG. 27A) and guide the pin member 388 into a groove or track 392 defined by each spool 382 and connected along the length of the spool member 380. The rod 386 may be rotated (see arrow R1 in FIG. 27B) until the pin member 388 hits the first end 392a of the portion of the track 392 defined by the first spool 382a (FIG. 27C), which begins to rotate the first spool 382a, as the other spools 382 remain stationary, and deploys the first CT. The rotation of the rod 386 may then be reversed until the bore pin member 388 reaches the opposite end 392b of the track 392 (FIG. 27D), after which point continued rotation of the rod 386 may catch the suture spool and retract the suture, completing the implant deployment process. During the next trigger pull, the central bore may pick up the next spool 382b and repeat the process. Because this design only moves one CT at a time during the deployment process, the risk of a CT getting caught when not being deployed is advantageously reduced. This configuration may also lack openings and complementary flexure plugs (and similar components). The number of independently rotatable spools or spool portions may vary. Examples may include one rotatable spool or two or more spools, including three, four, five, six, seven, eight, nine, ten spools, or more.

As shown in FIGS. 18-27D, delivery devices and systems disclosed herein can deploy multiple implants from a single delivery device pre-loaded with the implants. Embodiments can include a handle assembly that houses a spooling mechanism. The handle assembly can include a first spool member (e.g., suture spool member 302) configured to couple with multiple implant assemblies simultaneously, e.g., multiple CT/suture assemblies. The handle assembly can also include a manifold member (e.g., manifold member 306) or assembly (e.g., manifold spool assembly 300). The manifold member can be coupled with the first spool member and configured to receive the CT/suture assemblies in serial fashion, i.e., one-by-one. The manifold may guide or direct the CT/suture assemblies toward and into the lumen of a needle configured to pierce a prostate gland, the needle coupled to a second spool member (e.g., needle spool member 308) configured to deploy the needle. The second spool member may be attached to or coupled with a plurality of movable (e.g., pivotable, slidable, rotatable) components, such as flexure plugs (e.g., flexure plugs 310), which may be biased toward a closed position in which the CT/suture assemblies are prevented from unwinding from the first spool member, through the manifold, and into the needle. Accordingly, the implants or components thereof can be detached (e.g., unwound) from the internal spool member(s) of the handle assembly in a controlled fashion at the direction of a user via engagement with an actuator (e.g., actuator 354). Related embodiments may include a rotatable cam component or indexer (e.g., cam indexer 328) configured to engage the movable components (e.g., flexure plugs 310), again in serial fashion, such that rotation of the cam indexer causes the movable components to transition to an open position in serial fashion, thereby allowing passage of the CT/suture assemblies into the needle one-by-one. The manually engageable actuator, e.g., trigger, configured to initiate the unspooling and deployment of the implants and components thereof may be biased (e.g., spring-loaded), and may be coupled directly or indirectly to one or more of the internal spool members, for example via one or more gearing features within the handle.

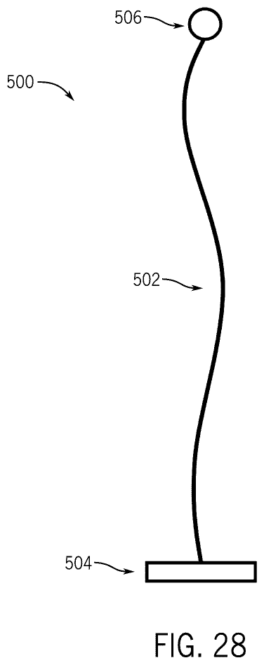

One or more of the aforementioned components, assemblies, subassemblies, or mechanisms may be modified, reconfigured, removed, replaced, and/or combined with additional embodiments of a reloadable and/or multi-fire delivery device disclosed herein. For example, FIGS. 28-34H depict a multi-fire device configured to simultaneously hold and serially deploy multiple implants using an auto-indexing magazine assembly in conjunction with a variety of additional components.

FIG. 28 depicts an example of a CT/suture assembly 500, which may be compatible with one or more of the delivery devices and systems disclosed herein, including a delivery device featuring an auto-indexing mechanism, e.g., an auto-indexing magazine assembly. As shown, the CT/suture assembly 500 (or implant) may include an elongate connector portion or suture 502 connected at one end to a CT 504 and at the opposite end to an enlarged portion 506, non-limiting examples of which may include or define a bulbous tip or ball member or something similar configured to be grabbed, grasped, ensnared, or otherwise engaged by another component of the delivery device. In some embodiments, the enlarged portion 506 provides a UE. The CT 504 may be configured in a variety of sizes and shapes, for example resembling a T-bar. The enlarged portion 506 is positioned at the proximal end of the assembly 500 when loaded into a delivery device, where it can be grabbed by a component of the delivery device and pulled proximally to tension the suture 502 after implantation. The CT/suture assembly 500 may be a single, unitary component comprised of a variety of materials, e.g., a molded polymer. Alternatively, the CT/suture assembly 500 may be comprised of two or more discrete components, for instance such that the CT 504 is distinct from the enlarged portion 506. The CT/suture assembly 500 may be advanced distally, through the lumen of a delivery needle and ultimately beyond the distal tip of the needle by a longitudinal push member, examples of which are described below, which is then retracted and the ball member pulled proximally to achieve tensioning. Due to the positioning and manner in which the CT/suture assembly 500 is indexed and delivered in this embodiment, each suture 502 may be referred to as a “suture tail.”

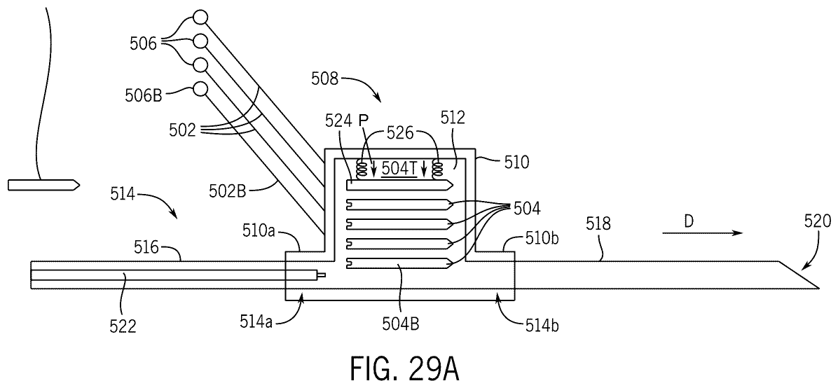

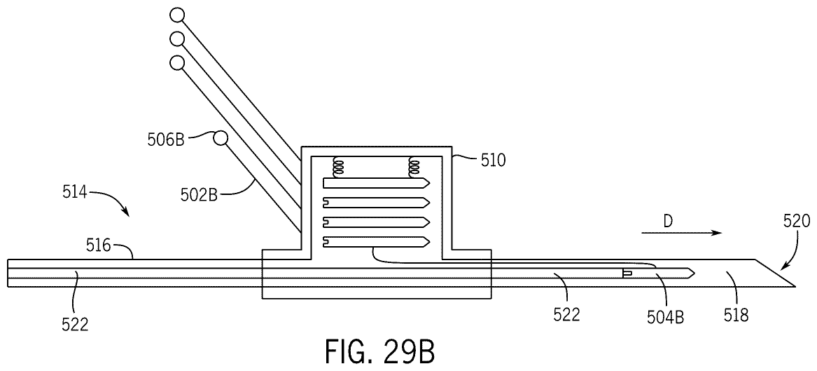

FIGS. 29A and 29B provide cross-sectional views of a needle assembly 508 configured to simultaneously hold and index multiple implants during a treatment procedure. As shown, the needle assembly 508 may include an indexing magazine assembly 510 defining an internal cavity 512 configured to contain multiple CTs 504, each formed integrally with or attached to a suture tail 502. The needle assembly 508 may include a multi-part needle, such as a two-part needle 514 comprising a first, proximal needle portion 516 and a second, distal needle portion 518, which may include a beveled distal tip 520. The needle assembly 508 may be coupled with an elongate push member 522 that extends through the lumen defined by the two-part needle 514. The push member 522 may move independently of the needle assembly 508 as the push member 522 pushes the auto-indexed CTs 504, in serial fashion, in the distal direction D during a multi-implant deployment process. The suture tails 502 are pulled along via their attachment (or formation with) the CTs 504 being pushed by the push member 522.

As further shown, the proximal end 510a of the magazine assembly 510 may be attached to the distal end 514a of the first, proximal needle member 516, and the distal end 510b of the magazine assembly 510 may be attached to the proximal end 514b of the second, distal needle member 518, with a portion of the cavity 512 defined therebetween. The CTs 504 may be stacked within the cavity 512, one on top of the other, with the suture tails 502 extending through a slot, opening, gap, or cutout portion in the rear (proximal) side of the magazine assembly 510. The cavity 512 may further include a spring-loaded feature or plunger member 524 biased to push downward, in the direction of the arrows in FIG. 29A, against the top CT 504T. One or more springs 526 or other biasing members may drive this downward pushing force.

The lowest or bottom CT 504B may be longitudinally aligned with the two-part needle 514, positioned between the proximal needle member 516 and the distal needle member 518. To advance the bottom CT 504B through the two-part needle 514, toward a targeted prostatic lobe, the push member 522 is advanced distally, pushing the bottom CT 504B from its proximal end. Via the push member 522, the bottom CT 504B is pushed into the second, distal needle portion 518, pulling the bottom suture therewith, as shown in FIG. 29B. The bottom CT 504B may be pushed beyond the distal tip 520 of the distal needle portion 518 and into the targeted lobe of the prostate, after which the push member 522 may be retracted to the position shown in FIG. 29A, ready to push the next CT. The bottom-most enlarged portion 506B may be grasped and retracted proximally, thereby tensioning the suture 504B attached thereto. A UE may by attached to the tensioned suture 504B at the urethral side of the lobe and the surplus suture cut and removed.

With the push member 522 fully retracted to a position proximal to the magazine assembly 510 and the excess suture removed, the spring-loaded plunger 524 may automatically push the next CT downward until it is longitudinally aligned with the first, proximal needle portion 516 and the second, distal needle portion 518. The CT/suture delivery process may then be repeated. Accordingly, the needle assembly 508 may be configured to automatically index successive implants for serial deployment into the tissue targeted during a treatment procedure.

Because the magazine assembly 510 may be in-line with and integral to the two-part needle 514 in this embodiment, the magazine assembly 510 may move with the needle 514 during its distal advancement and proximal retraction. This integration of the magazine with the needle may simplify the system as a whole by eliminating the need to monitor, track, or otherwise account for the various positions of the needle during the deployment sequence.

The number of CT/suture assemblies simultaneously included in the magazine assembly 510 of the needle assembly 508 may vary. The example shown includes four CTs 504 in the magazine cavity 512, but embodiments can include fewer or more than four CTs, including one, two, three, five, six, seven, eight, nine, ten CTs, or more.

The needle assembly 508 may be integrated, attached, formed with, or otherwise coupled with a compatible handle assembly configured to transfer mechanical energy to the needle assembly 508 via manual activation by a user. The handle assembly may share one or more components or assemblies with other handle assemblies disclosed herein.

As shown in the example illustrated in FIGS. 29A and 29B, embodiments of an implant delivery device configured to simultaneously store and serially deploy multiple implants may include an indexing mechanism. The indexing mechanism can index one or more components of an implant, including the CT, suture, and/or UE, one or all of which may be integrally formed or attached in a single, unitary implant (e.g., implant 500). Examples of an indexing mechanism may include a magazine assembly (e.g., magazine assembly 510) configured to simultaneously hold multiple CTs (e.g., CTs 504), each of which is attached to or formed with a suture (e.g., suture 502). A needle assembly (e.g., needle assembly 508) can be attached or coupled with the magazine assembly. The associated handle assembly can include an actuator (e.g., trigger) and an elongate push member (e.g., push member 522), which may comprise a slidable bar component and which may move independently of the magazine assembly. The push member can advance longitudinally through the needle assembly in response to user-activation of the actuator, for example via trigger pull. Examples of the magazine assembly can include a spring-loaded plunger element (e.g., plunger member 524) or other biased feature that urges or moves each CT, one-by-one, into longitudinal alignment with the long axis of the needle assembly, for example between a first, proximal segment of the needle assembly (e.g., proximal needle member 516) and a second, distal segment of the needle assembly (e.g., distal needle member 518), thereby indexing the implants during serial deployment thereof. Embodiments may thus include segmented or multi-part needles, which may be configured to receive CT/suture assemblies in the gap(s) between the segments. The elongate push member advances each implant through the needle assembly, one-by-one, through direct or indirect contact with each implant or portion thereof, e.g., CT. The needle assembly and magazine assembly coupled therewith may move in unison, while the elongate push member moves independently in response to user activation of the actuator, e.g., trigger pull.

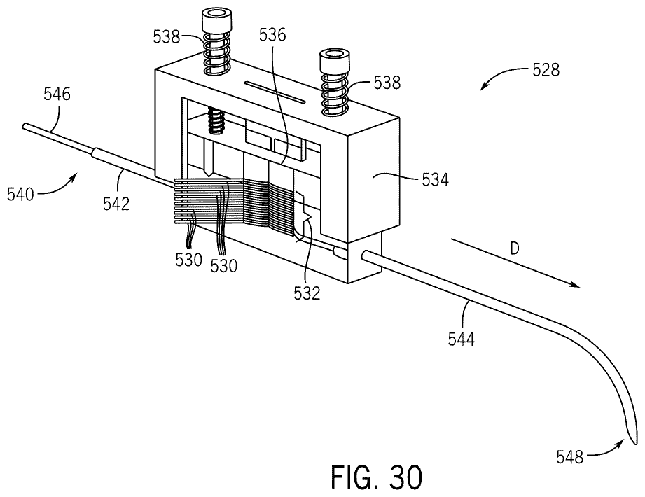

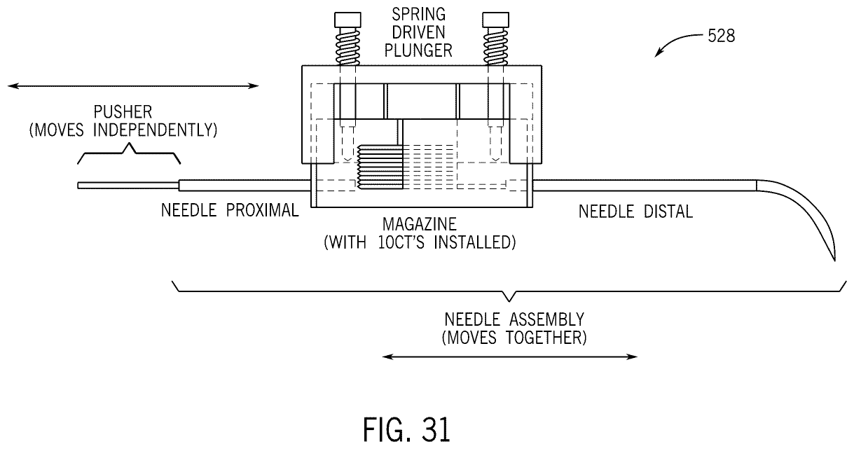

FIGS. 30 and 31 provide a perspective and transparent side view, respectively, of a needle assembly 528 holding ten CT/suture assemblies 530, the CTs 532 of which may be stacked vertically within a magazine assembly 534. The CTs 532 may be urged downward within the magazine assembly 534 via a spring-loaded plunger element 536, for example, which is attached to one or more biasing elements, e.g., springs 538, in the particular embodiment shown. The bottom-most CT may be aligned, via the plunger element 536, with a multi-part needle 540, which may include a first, proximal needle portion 542 and second, distal needle portion 544. An elongate push member 546 may be configured to extend through the inner lumen defined by the multi-part needle 540. The number of CT/suture assemblies 530 may vary. In the embodiment shown, the needle includes two needle portions or segments, and may thus be referred to as a two-part needle.

In operation, the needle assembly 528 may be deployed forward, in the distal direction D in response to user engagement with an actuator, such that a distal needle portion 548 approaches the prostatic urethra. The bottom-most CT, longitudinally aligned with the two-part needle 540, may be pushed distally by the push member 546 to advance the CT/suture assembly through the two-part needle 540 toward the needle's distal tip 548, where the CT/suture assembly can be pushed through and beyond the distal tip 548 after the tip pierces the prostatic capsule, or alternatively the CT/suture assembly can be unsheathed by the distal needle portion 544 upon retraction of the needle 540. Suture tensioning, UE seating, and suture cutting may then be performed via one or more mechanisms disclosed or incorporated by reference herein, which may involve the activation of one or more components of the handle assembly. The push member 546 may then be retracted proximally, and the next CT indexed downward by the spring-loaded plunger element 536.

Needle assemblies 508 and 528 are configured to provide a long-suture multi-fire mechanism by providing a means to compactly store multiple CT/suture assemblies and index those CT/suture assemblies into place within the delivery needle. The compact size and the integration of the magazine into the needle allow the indexing mechanism to move with the needle, thus eliminating or reducing challenges associated with aligning an indexer to the needle when the needle needs to move back and forth. This long-suture multi-fire mechanism enables the loading and alignment of multiple CT/suture assemblies into one delivery device simultaneously. By using a pushing element to advance each CT (and drag the suture behind it), needle assemblies 508/528 may depart from the preexisting devices configured to advance a CT by pushing the suture, thus eliminating concerns or constraints about the column strength of the suture itself. By employing a long-tailed suture, the disclosed embodiments also allow the pushing (advancing down the needle, distally) to be separate from the tensioning (pulling backward, proximally). This eliminates the need for the pusher to connect to or hold onto the CT. Tensioning can be achieved separately by simply pulling on the back end of the long suture.

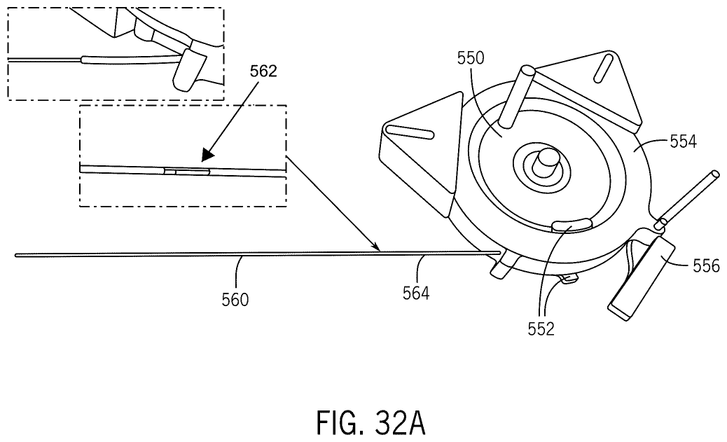

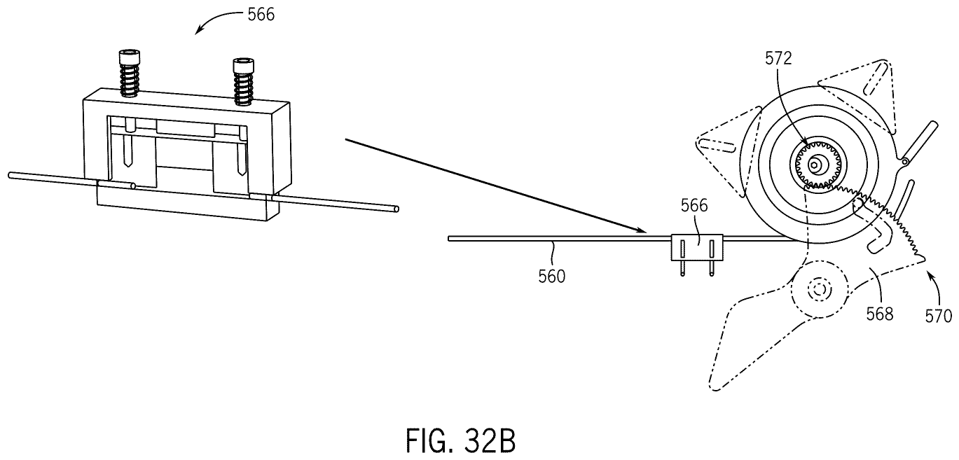

The auto-indexing mechanism implemented via the magazine assembly shown in FIGS. 29A-31 may be incorporated into, coupled with, or otherwise compatible with a variety of handle assemblies or components thereof, including embodiments configured to implement a rotary based system. FIG. 32A shows an example of certain components that may be included in a rotary based system configured to transfer mechanical energy to an auto-indexing needle assembly featuring a magazine component pursuant to a multi-implant deployment process. As shown, the handle assembly of the system may include one or more spool members, including three spools, which may be concentrically arranged, e.g., in a nested configuration, in certain non-limiting examples. The illustrated embodiment includes a pusher spool member 550, a needle spool member 552, and a tensioning spool member 554, which may be included together in a handle assembly with a tensioning pawl 556 and a suture engagement member (suture grabber 558) configured to capture, ensnare, or otherwise grab a suture. An example of a needle 560 utilized in this embodiment may include a slot 562 configured to receive the CTs. A pusher member 564 extends longitudinally from the body of the handle assembly into the needle 560.

FIG. 32B shows the handle assembly components, along with an indexing magazine assembly 566 mounted on the needle 560. Magazine assembly 566 may be the same or similar to magazine assemblies 510 and 534. An actuator 568 (i.e., a trigger in this embodiment) having gear teeth 570 configured to interact with a gearing portion 572 of the pusher spool member 550 may also be included in the handle assembly. Activation of the actuator 568 (e.g., trigger pull) may rotate the pusher spool member 550, advancing the pusher member 564 and implant down the elongate shaft assembly.

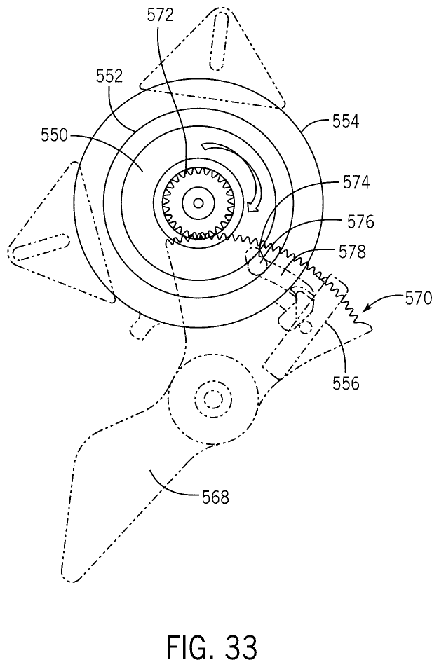

FIG. 33 provides another illustration of components of the handle assembly, further showing a trigger/needle spool cam path/boss interaction 574, featuring a boss member 576 and a cam pathway 578. This trigger-driven embodiment utilizes a geared trigger 568 to interact with the pusher spool member 550. The gearing may be configured such that a reasonable trigger stroke for a hand-held surgical device results in enough spool rotation to drive the implant from the delivery handle through the elongate shaft assembly and out of the distal end of the needle 560. The trigger 568 may also interact with the needle spool member 552 such that the needle 560 is fully extended prior to the CT being unsheathed or pushed out of the needle 560. The needle spool member 552 may be driven by the boss 576 on the spool which interacts with the cam pathway 578 on the trigger 568. The out-sweep of the trigger 568 may then retract the needle 560 and the pusher spool member 550 simultaneously. At the end of the out-sweep, the trigger 568 may interact with the tensioning pawl 556, allowing the tensioning spool member 554 to apply tension to the suture. This interaction may be indirect, as the trigger 568 drives the needle spool member 552 back to a position where the needle spool member 552 un-latches the tensioning pawl 556. After the trigger out-sweep, the tensioning spool member 554 may then need to be re-set, for example via manual intervention, or via coupling with a resetting action implemented during the urethral endpiece delivery process.

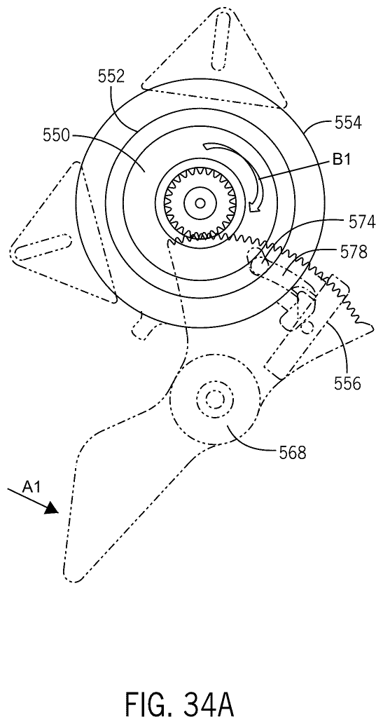

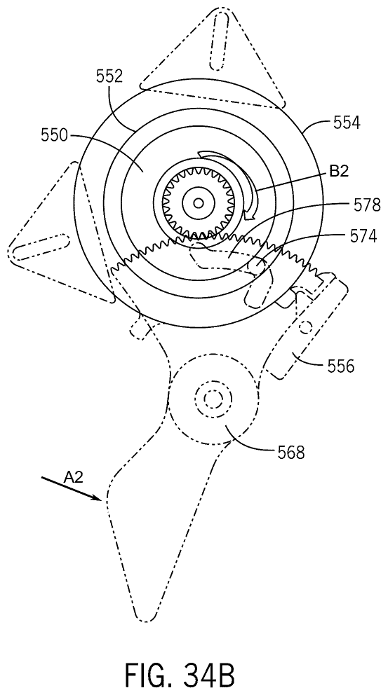

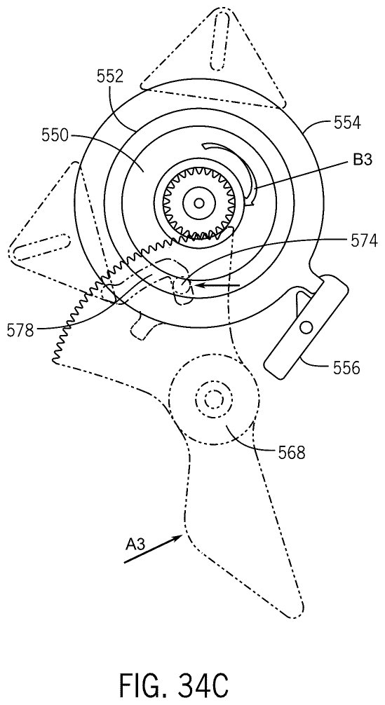

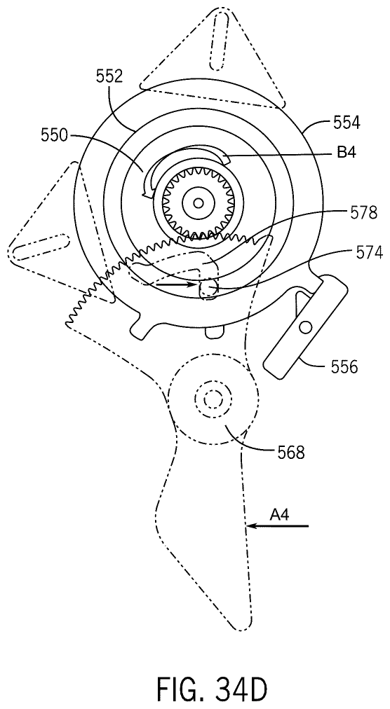

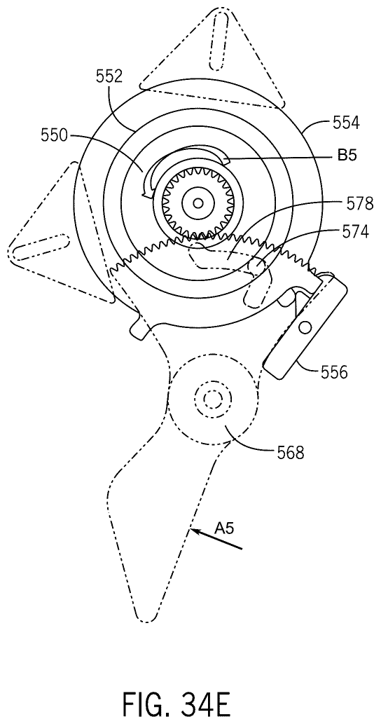

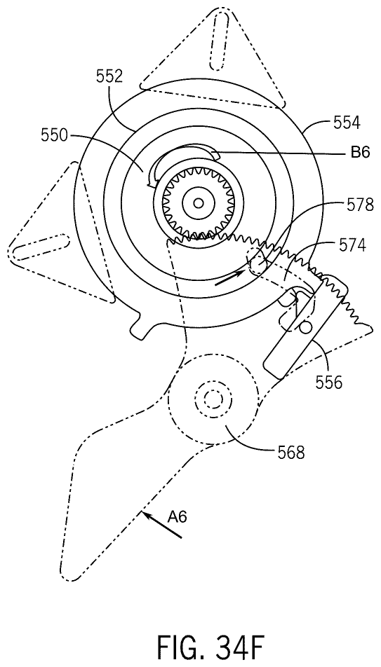

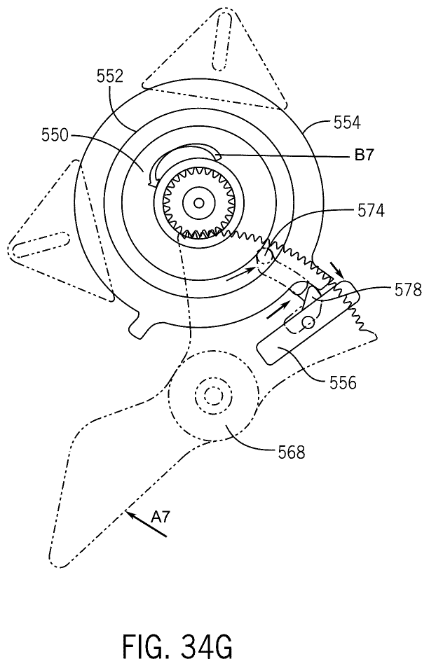

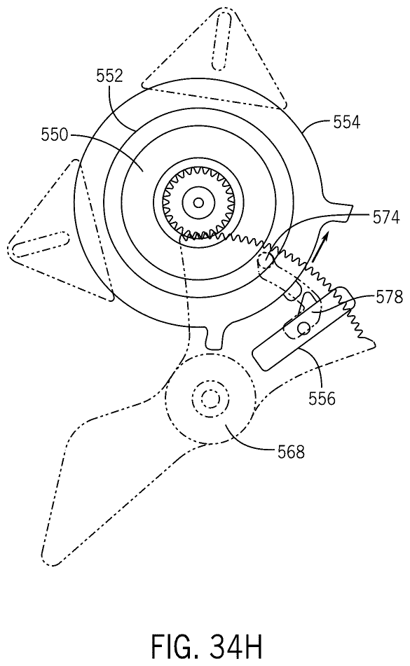

FIGS. 34A-34H illustrate snapshots of operatively coupled components of the handle assembly configured for use together with the magazine assembly 566 during CT/suture deployment. As shown in FIG. 34A, as the trigger 568 is moved (e.g., as shown by arrow A1 in FIG. 34A) a trigger pull may rotate the pusher spool member 550 (e.g. in the direction indicated by arrow B1 in FIG. 34A), which advances the pusher member and implant down the shaft assembly. Continued trigger pull (arrow A2) continues to rotate the pusher spool member 550 (arrow B2) and advances the implant, without causing rotation of the needle spool member 552 (FIG. 34B). As the continued trigger pull (arrow A3) further rotates the pusher spool member 550 (arrow B3) and advances the implant, the cam pathway 578 in the trigger 568 interacts with the boss member 576 on the needle spool member 552, causing rotation and advancement of the needle 560 through the shaft assembly (FIG. 34C). The subsequent trigger out-sweep (arrow A4) causes counter rotation of both the pusher spool member 550 (arrow B4) and the needle spool member 552 (FIG. 34D). Continued trigger out-sweep (arrow A5) continues to counter-rotate the pusher spool member 550 (arrow B5) and retract the pusher member 564 and the needle spool member 552 falls into the neutral cam path on the trigger 568, at which point no counter rotation can occur (FIG. 34E). Near the end of the trigger out-sweep (arrow A6), the needle boss member 576 may be rotated by the end of the cam pathway 578 on the trigger 568, causing slight additional counter rotation (arrow B6) (FIG. 34F). Final counter rotation of the pusher spool member 550 (arrow B7) and the needle at the end of the trigger out-sweep (arrow A7) disengages the tensioning pawl 556 (FIG. 34G). Disengagement of the tensioning pawl 556 allows for the spring-energized tensioning spool member 554 to be rotated (FIG. 34H). Resetting of the device may then be required. After tensioning, counter-clockwise rotation of the trigger 568 for the next deployment interacts with the tensioning spool member 554 to automatically reset by rotating the tensioning spool member 554 clockwise, which extends the tensioning spring and allows the spool to re-engage the tensioning pawl 556 to hold in the ready-to-tension position.

One complication that may occur during an implant delivery procedure is a CT pull-through event in which an implanted CT pulls back through the capsular surface of a prostate gland and into the prostatic tissue upon tensioning the suture. This type of failed deployment may not only damage the tissue, but jam the delivery device when the deployed CT is pulled back into the elongate shaft assembly (e.g., shaft assembly 104). Jammed devices disrupt the procedure and often require scrapping the entire device or portion thereof, such as the cartridge in embodiments featuring the same. Pull-through events may be especially problematic during the deployment of multiple implants, for example using one or more of the reloadable or multi-fire delivery devices disclosed herein. Damage to a multi-fire device may prevent multiple implants from being deployed from the damaged device, as the device and remaining implant(s) included therein may need to be scrapped.

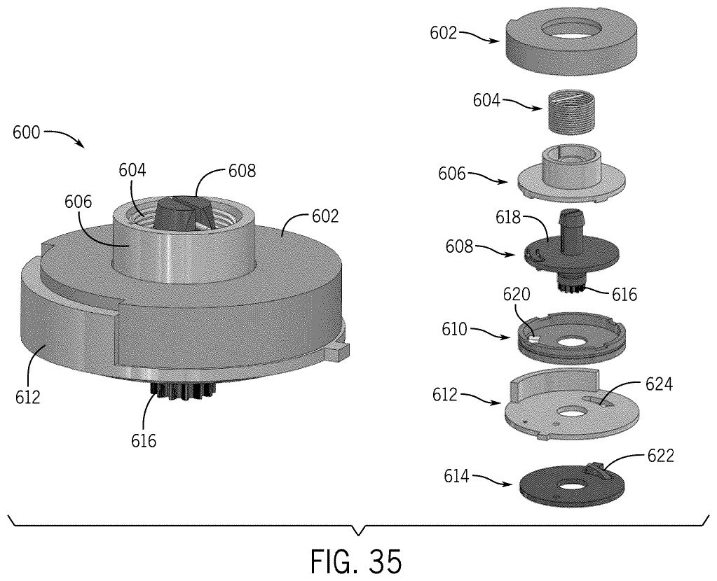

A device assembly 600 and mechanism for recovering CT/suture assemblies and preventing jamming after a pull-through event is shown in FIG. 35, with the individual assembly components shown in the exploded view on the right. In the illustrated embodiment, the assembly 600 includes a suture housing 602, a tension spring 604, a spring housing 606, a tension spool member 608, a suture spool member 610, a needle spool member 612, and a caliper spool member 614. In some embodiments, the needle spool member 612 and caliper spool member 614 may be included in the handle assembly of a delivery device, while the suture housing 602, tension spring 604, spring housing 606, tension spool member 608, and/or suture spool member 610 may be included in a removable cartridge configured to couple with the handle assembly. The position of one or more components relative to each other and the handle/cartridge assembly may vary. One or more components of the associated handle and/or cartridge assembly may be the same or similar to those disclosed herein.

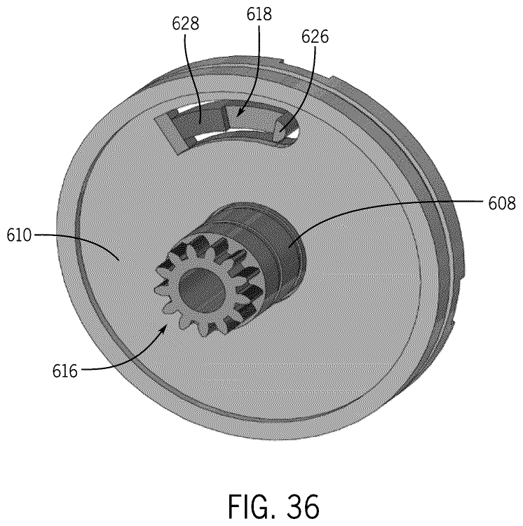

As further shown in FIG. 35 and FIG. 36, the tension spool member 608 may include a gearing portion 616 and a one-way tension spool flexure 618, the flexure configured to extend through a complementary, suture spool opening, cutout, or slot 620, which may be curved. The caliper spool member 614 similarly includes a one-way caliper spool flexure 622, which is configured to extend through a complementary opening, cutout, or slot, such as curved needle spool slot 624. Depending on their configuration or state during an implant deployment procedure, the flexures may allow or impede relative rotation of one or more spool members shown in FIG. 35, thereby facilitating or preventing advancement or retraction of a CT/suture assembly.

The suture housing 602 may be configured to constrain each CT/suture assembly against the outer surface of the suture spool member 610 around which the CT/suture assembly is wound to prevent CT/suture unraveling. The suture housing 602 may also provide a transitional lumen between the suture spool member 610 and the needle spool member 612, facilitating passage of the CT/suture from the cartridge to the handle assembly in this embodiment.

The tension spring 604 provides suture tensioning as the suture is being retracted following placement of the CT at the outer capsular surface. The tension spring 604 may also tie rotation of the tension spool member 608 with the suture spool member 610 and spring housing 606. The spring housing 606 mates and rotates with the suture spool member 610, driven by the tension spool member 608 in conjunction with the tension spring 604. The tension spool member 608 may be driven by an actuator (e.g., actuator 202), for instance via a gearing mechanism comprising the gearing portion 616 of the tension spool member 608 and complementary gear teeth on the actuator. Rotation of the suture spool member 610 may be driven by the tension spool member 608 in conjunction with the tension spring 604. A suture may wrap around and connect to the suture spool member 610, which may mate and rotate with the spring housing 606. The needle spool member 612 is attached to a needle (e.g., needle 162). Rotation of the needle spool member 612 may be tied to that of the suture housing 602.

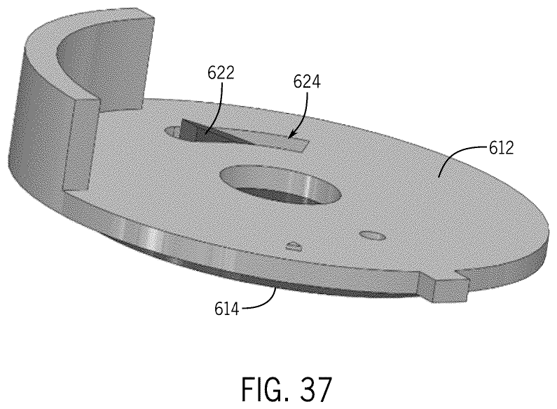

As further shown in FIG. 36, the one-way flexure 618 of the tension spool member 608 may define a protruding hard-stop portion 626, e.g., when encountered from the counter-clockwise direction, and a ramped portion 628, e.g., when encountered from the clockwise direction. FIG. 37 provides an illustration of the needle spool member 612 coupled with the caliper spool member 614. As shown, the one-way caliper spool flexure 622 may comprise an angled ramp portion configured to provide a hard stop, e.g., when encountered from the clockwise direction.

In operation, the caliper spool flexure 622 may be flexed down or otherwise against a surface of the needle spool member 612 in a non-obstructing configuration (i.e., out of the way), when the needle spool member 612 is fired to advance the needle to and beyond the distal end of an elongate member of the delivery device. The caliper spool flexure 622 may then pop, spring, or otherwise extend back through the needle spool slot 624 when the needle is fully retracted, thus re-assuming its obstructing configuration. Additionally, the caliper spool flexure 622 may be flexed down into the non-obstructing configuration when a manual release is triggered. For example, in the event of a CT pull-through, the caliper spool flexure 622 may stall the tension spool member 608 via interaction between the caliper spool flexure 622, in its obstructing configuration extending through the needle spool slot 624, and the hard-stop portion 626 of the tension spool flexure 618 extending through the tension spool slot 620. After the suture has been cut and the CT removed to prevent jamming of the device, the caliper spool member 614 can be rotated clockwise to flex the caliper spool flexure 622 back out of the needle spool slot 624 and against the lower surface of the needle spool member 612, in its non-obstructing configuration, where it no longer impedes movement of the tension spool member 608.

To unspool and deploy a CT/suture, the tension spool flexure 618 may drive the suture spool member 610 and spring housing 606, e.g., in the counterclockwise direction. Then, when the tension spool member 608 is rotated back, e.g., in the clockwise direction, to retract/tension the suture, if the tension in the suture spring 604 does not meet or exceed a specified threshold, the suture spool member 610 may rotate together with the tension spool member 608, indicating a pull-through has occurred, and resulting in the tension spool flexure 618 remaining up, extended through the suture spool slot 620 in an obstructing configuration. In this configuration, the tension spool member 608 may continue to rotate clockwise until the tension spool flexure 618 contacts the caliper spool flexure 622 extending through the needle spool slot 624, at which point further rotation of the tension spool member 608 and suture spool member 610, for example in the clockwise direction in this example, is prevented, thereby also preventing the CT and suture from retracting fully back into, and consequently jamming, the elongate shaft assembly of a delivery device. The suture may then be cut manually, the CT removed, and the caliper spool flexure 622 manually depressed to move it out of the way, as described above, to allow the suture to be retracted back into the suture spool member 610 without a CT attached thereto.

In contrast, if the level of tension in the suture spring 604 does reach the specified threshold when the tension spool member 608 rotates in the clockwise direction pursuant to suture retraction/tensioning, the tension spool member 608 and suture spool member 610 may separate, causing the tension spool flexure 618 to be pressed downward into its non-obstructing configuration. This indicates that a pull-through has not occurred, and the implant deployment sequence can continue.

Embodiments of the assembly shown in FIGS. 35-37 may be included in one or more delivery devices disclosed herein for deploying one or more implants to a targeted tissue. Embodiments of the assembly may also be included in one or more devices configured to deploy a single implant, for example one or more of the devices included in the references incorporated herein, which may feature a removable cartridge, and which may require removal of a shaft assembly from a patient during a procedure.

Accordingly, and as depicted in FIGS. 35-37, a delivery device configured to deploy multiple implants to a prostate gland without removing the elongate shaft assembly of the delivery device from a patient may include a handle assembly and removable cartridge together configured to recover a CT and attached suture in the event of a CT pull-through. The recovery mechanism may feature a spooling assembly, which can include multiple spool members. Specific embodiments can include four spool members, including a tension spool member (e.g., tension spool member 608), a suture spool member (e.g., suture spool member 610), a needle spool member (e.g., needle spool member 612), and a caliper spool member (e.g., caliper spool member 614). In some examples, the needle spool member and caliper spool member can both be included in the handle assembly, while the tension spool member and suture spool member can be included in the cartridge. A spring (e.g., tension spring 604) configured to tie rotation of the tension spool member with the suture spool member can also be included, where the tension spool member is driven by the actuator, e.g., trigger, on the associated handle assembly. The tension spool member can include one or more features configured to limit its rotation, for example by providing a unidirectional protrusion, component, or surface configured to allow movement (e.g., rotation) when encountered in one direction (e.g., clockwise) and prevent movement (e.g., rotation) when encountered in the opposite direction (e.g., counterclockwise). For instance, the tension spool member can include a hard-stop portion and a ramped portion. The configuration or state of one or more components can impact whether relative movement between certain components is allowed or prevented, and the configuration or state can be dictated by the positioning or deployment of one or more implant assemblies or components, such as a CT. For example, a one-way flexure on the caliper spool member can be flexed to a non-obstructing configuration when the needle spool member is rotated to fire a needle distally through the elongate shaft assembly of the delivery device. The one-way flexure can be configured to extend through a slot defined by the needle spool member after the needle is retracted, and in the event of a pull-through, the one-way flexure can prevent the tension spool member from rotating.

A variety of mechanisms may be employed to complete the final steps of the implant deployment process, for example by cutting an implanted suture and attaching or seating a UE thereto. Embodiments may involve pushing the UE onto a tensioned suture to seat the suture within the UE legs or prongs (see FIG. 4A) and subsequently pulling a cutter element into the suture to cut the suture and release the fully assembled implant from the elongate shaft assembly. Non-limiting examples of such UE cutting mechanisms are described in U.S. Pat. No. 11,298,115, the entire contents of which are incorporated by reference herein. Additional non-limiting examples are provided in U.S. Patent Application Publication No. 2021/0378658, also incorporated by reference in its entirety herein, for example beginning at paragraph 0085.

Disclosed herein are additional UE deployment and suture cutting mechanisms that may be included in one or more devices configured to deploy multiple implants during a single procedure without removing the associated elongate shaft assembly from the patient. Embodiments of such delivery devices may include a removable cartridge or one or more internal mechanisms, e.g., spooling assemblies, manifolds, and/or magazines, configured to store and serially deploy multiple implants during a procedure without removing the elongate shaft assembly from the patient.

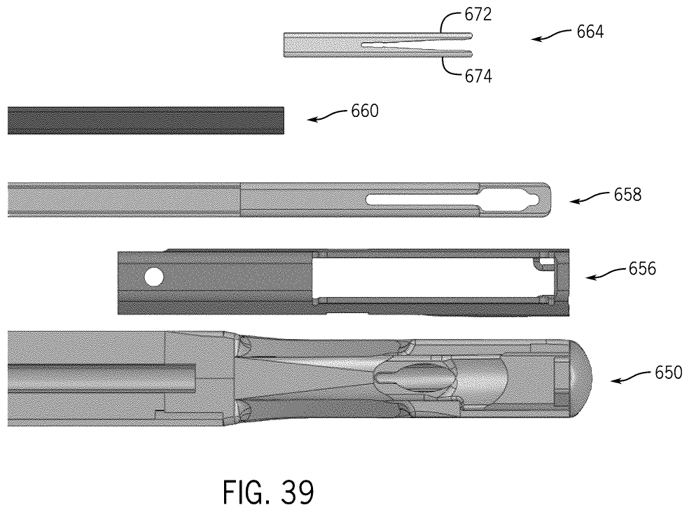

An example of a UE deployment and suture cutting assembly is shown in FIG. 38, illustrating a distal portion 650 of the shaft assembly 652 of an example of a delivery device configured to deploy a UE in a manner that involves pulling the suture into a stationary UE and subsequently cutting the suture at a proximal side of the seated UE. As shown, the distal portion 650 of the shaft assembly 652 may include a distal tip 654, along with at least a portion of a shaft member 656, a puller member 658, a backstop or pusher wire 660, a blade or cutting element 662, and a UE 664. The puller member 658 may include two apertures, looped portions or “lasso” members 666, 668 configured to surround a suture 670 and pull the suture 670 in the proximal direction in response to a pulling force applied to the puller member 658. The cutting element 662 may be positioned between the lasso members 666, 668, such that one lasso member may be considered the lower lasso member 666 and the other lasso member may be considered the upper lasso member 668. The components are shown separately in FIG. 39.

To attach the UE 664 to the suture 670, the UE 664 may be held stationary as the puller member 658 pulls the suture 670 into the UE 664 to seat the suture 670. The UE 664 may be held in the same orientation throughout the process, proximal to the suture 670 with the UE prongs 672, 674 extending or pointing distally, toward the suture 670. The position of the UE 664 may be maintained by the pusher wire 660, which may thread through the puller member 658. The cutting element 662 may remain stationary, fixed to the shaft member 656, during its attachment to, and cutting of, the suture 670. After attaching or seating the UE 664 on the suture 670, the puller member 658 may be pulled proximally, thus also pulling the suture 670 within the lasso members 666, 668 and against the sharp distal edge of the cutting element 662, which then cuts the suture 670 and releases the assembled implant.

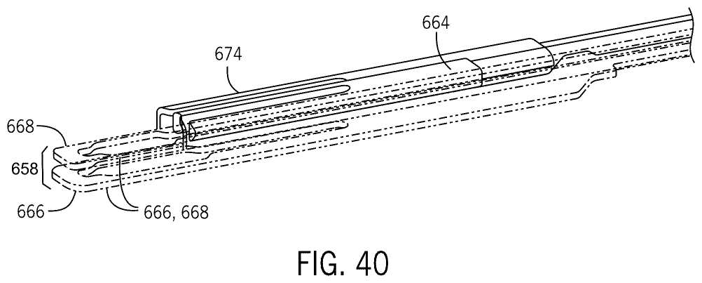

FIG. 40 provides an isolated view of the puller member 658, showing the UE 664 positioned within an enclosed portion 674 of the puller member 658. The number and configuration of the lasso members may vary. In the example shown, the lasso members 666, 668 thread above and below the cutting element 662 to provide an equal pulling force against the suture 670 on both sides of the cutting element 662, thereby ensuring clean, reliable suture cutting. The enclosed portion 674 may retain the UE 664 within the shaft member 656 until ejection of the UE is desired. At the completion of suture cutting, proximal movement of the puller member 658 may unsheathe the UE 664 just after suture cutting to allow ejection of the implant when the suture cut is complete.

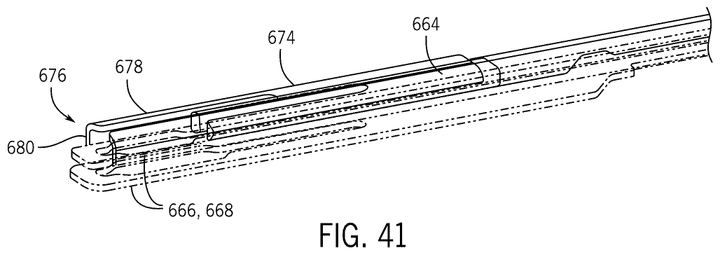

An additional embodiment of the puller member 658 may include a third lasso member, which may also be positioned above the UE 664, to facilitate pulling the suture into the UE 664 with greater force. The third lasso member may be releasable to allow the suture 670 and implant to release after the suture is cut. In some examples featuring a third lasso member, the configuration of one or more lasso members may include two opposing hooks instead of a continuous loop to enable release of the suture 670 at the appropriate time during UE deployment. For instance, as shown in FIG. 41, a third lasso member 676, which may also be considered an upper lasso member in the orientation shown, may comprise releasable hooks 678, 680 configured to aid with seating the suture 670 within the UE 664.

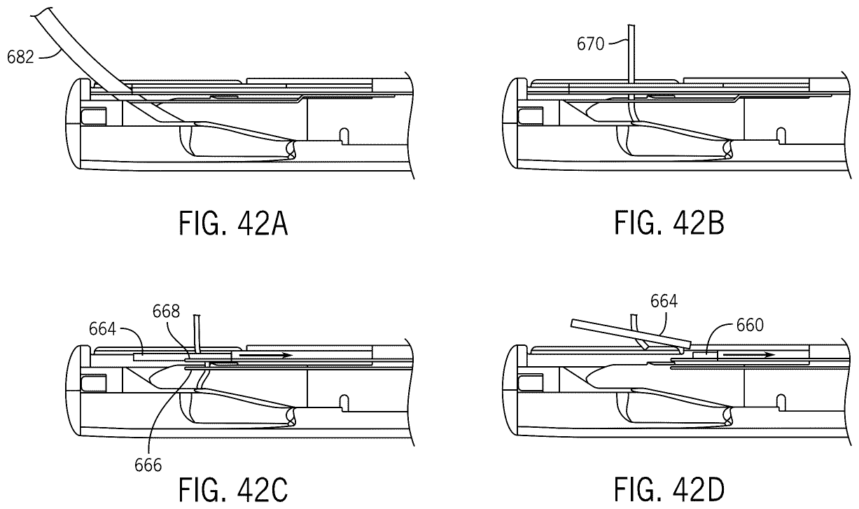

FIGS. 42A-42D illustrate snapshots of the distal portion 650 of the shaft assembly 652 during deployment of the UE 664 in accordance with this embodiment. As shown in FIG. 42A, a delivery needle 682 having an inner lumen through which the suture extends may be advanced away from the device, into the targeted prostatic tissue. The needle 682 may then be retracted proximally, back into the shaft member, thereby exposing the suture 670, which may be tensioned (FIG. 42B). The puller member 658 may then be pulled proximally, with the lower lasso member 666 and upper lasso member 668 pulling the suture 670 against the distal tip or edge of the fixed cutting element (FIG. 42C). Continued pulling of the suture 670 in the proximal direction results in cutting of the suture 670 on the fixed cutting element 662 and unsheathing of the UE 664, allowing ejection and complete release of the now-assembled prostatic implant (FIG. 42D).

Attaching a stationary UE to a suture in this manner may be driven by one or more components or subassemblies present within a handle assembly connected to the proximal end of the shaft assembly 652. In some embodiments, a spring, a pawl, and/or a block member present within the handle assembly may be operatively coupled to the UE deployment components shown in FIGS. 38-42D. For instance, the puller member 658 may be connected to a block member inside the handle assembly. The block member may be connected to the spring, which may be held in its energized state when ready to fire by the pawl. When triggered, the pawl may release the block member, thereby allowing the spring energy to propel the block and puller member to drive the disclosed implant formation sequence. Alternatively, the puller motion may be quasi-static, rather than dynamic, in which case a user may manually actuate a button/lever/slider to translate drive motion of the puller member.

As shown in FIG. 38-42D, delivery device embodiments can include an elongate shaft assembly that includes movable components configured to attach a UE to a suture and cut the suture thereafter. The UE may remain stationary during this process, along with a suture cutting element. As a result, cutting the suture can involve pulling the suture proximally into the sharp cutting surface of the distal end of the cutting element. The suture may be pulled in this manner by a moveable (e.g., slidable) puller member positioned within the shaft assembly. Examples of the puller member can include or define one or more apertures or hook-like features configured to surround or ensnare the suture. Pulling the suture in the proximal direction can attach the suture to the UE, followed by cutting of the suture just proximal to the attached UE.

One or more components of the handle assembly may be the same or similar to those shown and described in U.S. Pat. No. 11,298,115. The components and mechanisms of the UE deployment process described herein may be simplified relative to those featured in preexisting devices. The components and mechanisms may also be configured to be reset in a simplified manner when integrated into a multi-fire or reloadable device, such as those described above in connection with FIGS. 5A-37. Additionally, the length of the shaft and distal tip portion extending beyond the needle exist position may be reduced, which may improve the visibility and ease of maneuvering the device through the urethra and prostate.

Delivery components and assemblies configured to simultaneously store and successively deliver multiple UEs may be included together with device components and assemblies configured to simultaneously store and successively deliver multiple CT/suture assemblies without removing the elongate shaft assembly of an associated delivery device, which may include one or more delivery devices, assemblies, and/or components disclosed herein. Certain examples may include a UE deployment and indexing mechanism driven by movable components positioned within a shaft assembly and a handle assembly of a delivery device.

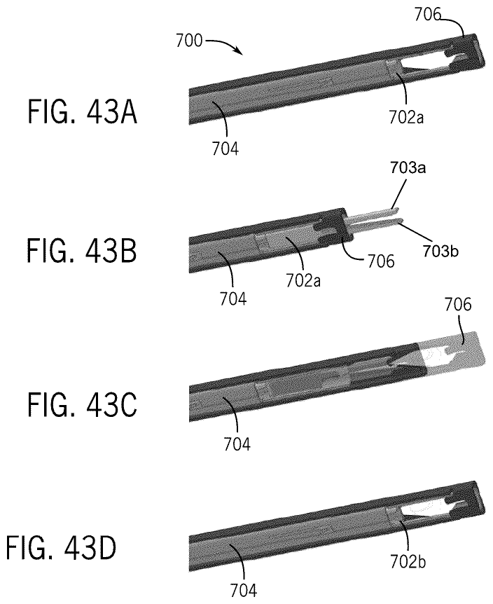

FIGS. 43A-43D illustrate snapshots of a distal portion of an embodiment of a shaft assembly 700 during the serial deployment of multiple UEs. The UE deployment mechanism implemented by the device may involve pulling an implanted suture proximally, into a UE, in response to manual actuation, e.g., trigger pull. Unlike the embodiment depicted in FIG. 38-42D, the cutting member, e.g., blade, of this embodiment may not remain stationary. Instead, the cutting member may be pushed distally into the suture, in some examples via spring action, thereby cutting the suture and ejecting the now-coupled UE. The mechanism may then be reset via manual actuation, e.g., trigger pull, which advances the next loaded UE forward, in the distal direction for deployment. In this manner, multiple UEs may be deployed pursuant to a procedure involving the deployment of multiple prostatic implants.

FIG. 43A depicts the distal portion of the shaft assembly 700 in a starting or first position, before ejecting the distal-most UE 702a, which may be considered the first UE in the illustrated multi-fire sequence. The suture remains distal to the first UE 702a, which includes distally extending prongs or leg members 703a, 703b, the majority of which are initially concealed by a movable channel 704 positioned within the shaft assembly 700. A cutter member 706 configured to cut the suture is coupled with the channel 704.

The channel 704 and cutter member 706 may then move proximally (FIG. 43B), pulling the suture in the same direction, into the first UE 702a. The cutter member 706 may then be pushed in the distal direction, severing the suture and releasing the first UE 702a, now attached to the suture (FIG. 43C). A movable pusher member positioned within the shaft assembly 700 may be configured to hold the first UE 702a in place during UE seating and suture cutting.

After deployment of the first UE 702a, the pusher member may advance the next UE, represented as UE 702b, into place at the distal end of the shaft assembly 700 for attachment to a separate suture (FIG. 43D), thereby forming a separate implant during the same procedure. The UEs 702a, 702b . . . 702ₙ may be stored axially within the shaft assembly and advanced forward (distally) for each subsequent deployment. The UE capacity of the shaft assembly may vary in different embodiments, such that the shaft assembly may simultaneously hold two or more UEs, including three, four, five, six, seven, eight, nine, ten UEs, or more, to match the number of corresponding CT/suture anchor assemblies

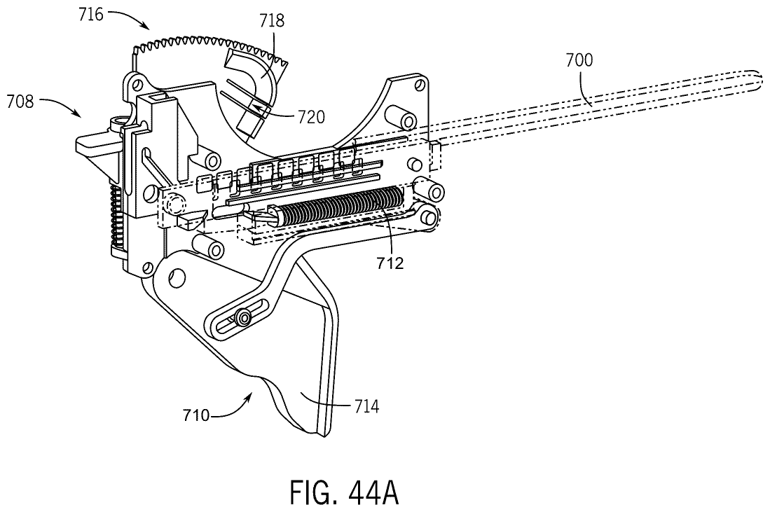

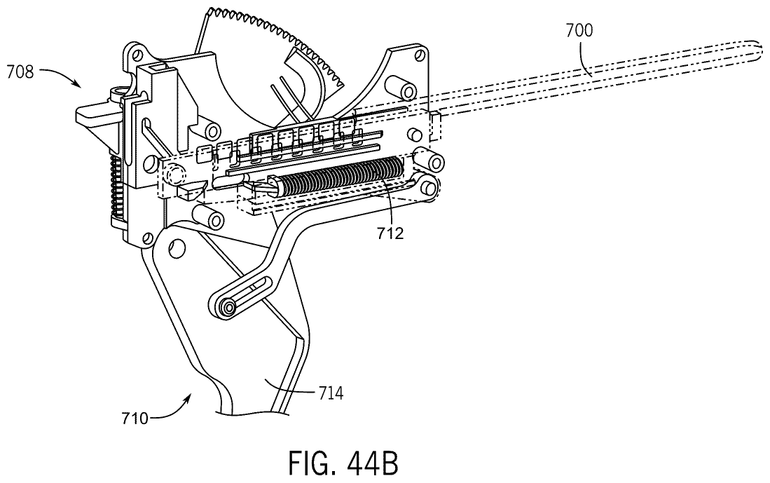

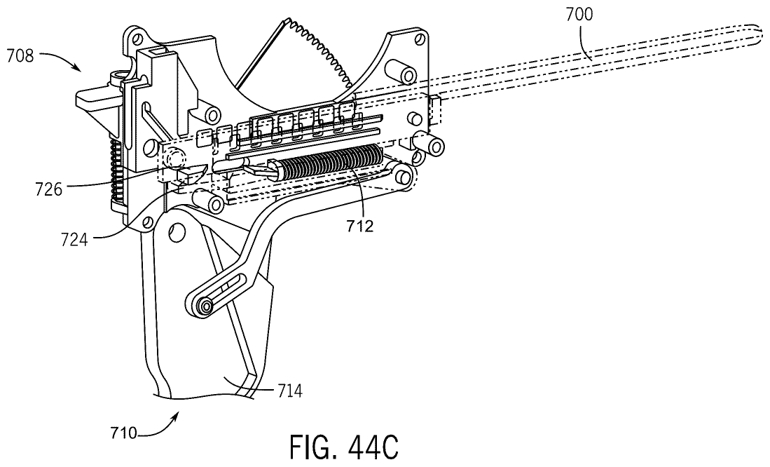

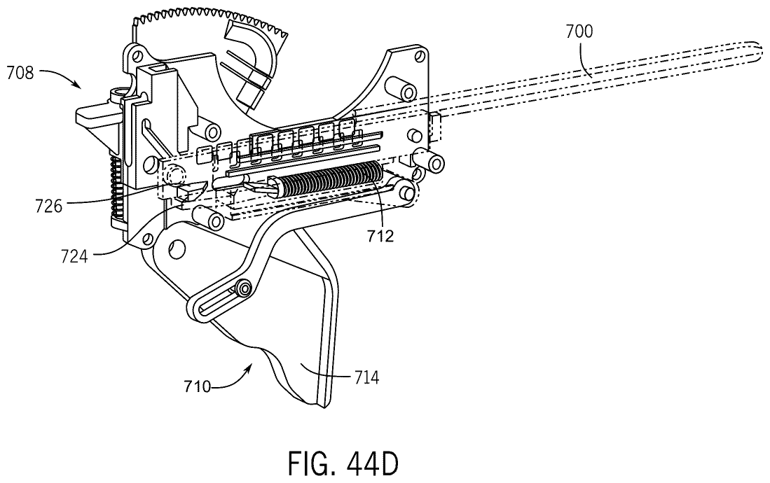

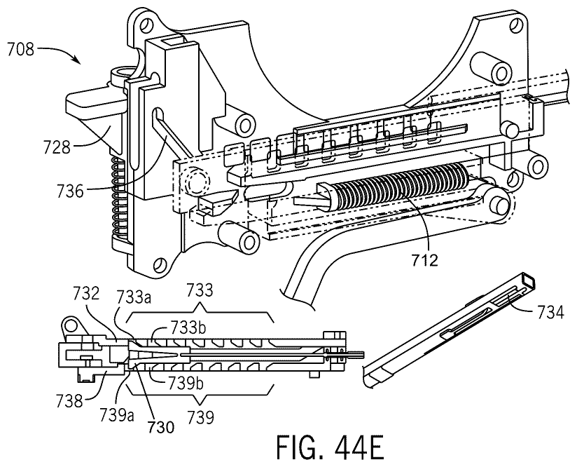

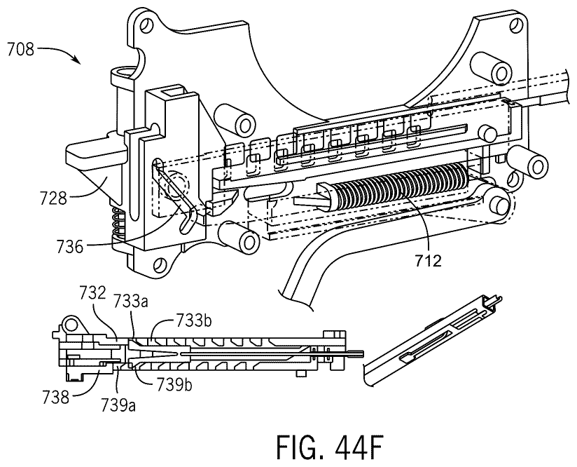

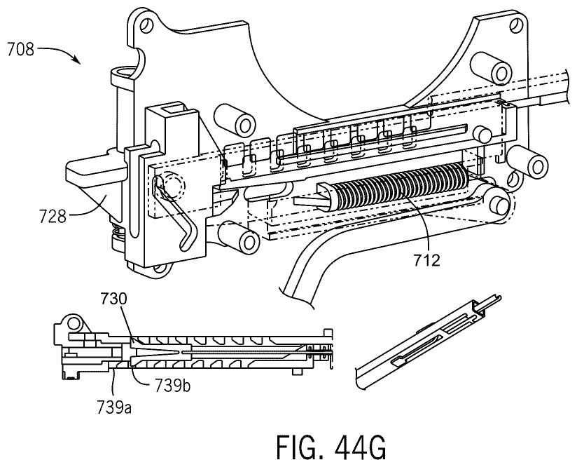

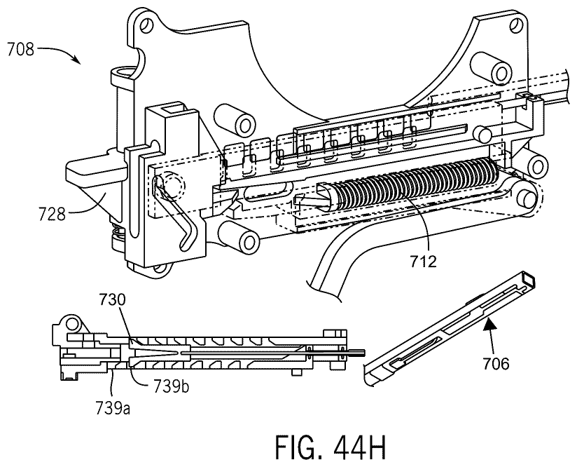

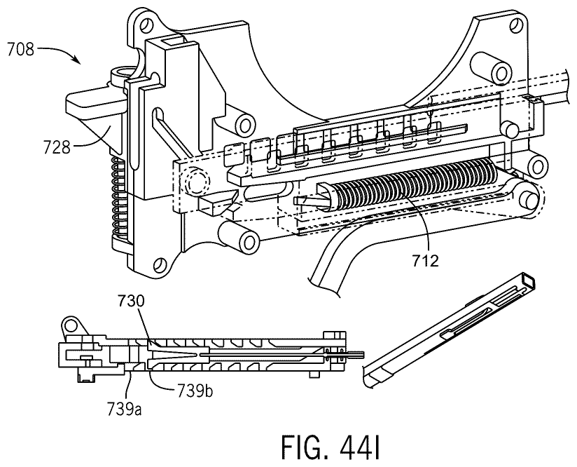

In some embodiments, the movable components within the shaft assembly, including the channel 704, cutter member 706, and pusher member, may be activated by an indexing mechanism within the handle assembly of the delivery device. FIGS. 44A-44I illustrate snapshots of an embodiment of the internal components of a handle assembly 708 during implementation of this indexing mechanism. As shown in FIG. 44A, the handle assembly 708 may include an actuator 710 (e.g., trigger) and an internal cutter spring 712. The handle assembly 708 may be similar, identical, or at least compatible with one or more additional handle assemblies shown and described herein. For instance, actuator 708 may include a manually engageable portion 714 and gear teeth 716, together with a protruding wall portion 718 and a flexure 720, like actuator 354 (see FIG. 24A).

FIGS. 44A-44D more particularly show the process of energizing a cutter spring 712 configured to move the cutter member 706 axially during successive UE deployments. As shown in FIG. 44A, the cutter spring 712 may be partially energized during the UE-suture assembly process. The manually engageable portion 714 of the actuator 708 is fully out, ready to be pulled inward by a user. After pulling the manually engageable portion inward, but not completely (e.g., ins-weep about 50%), energizing of the cutter spring 712 may not have begun (FIG. 44B). Complete or nearly complete in-sweeping of the manually engageable portion 714 may fully energize (e.g., compress) the cutter spring 712, at which point a snap arm member 724 may also engage a complementary ledge portion (or “catch”) 726 in the handle assembly 708, as shown in FIG. 44C. With the snap arm member 724 now engaged with the ledge portion 726, the cutter spring 712 may remain fully energized after full release of the manually engageable portion 714 (FIG. 44D).

FIGS. 44E-44I depict additional components of the handle assembly during the UE deployment process, working in combination to implement a UE indexing mechanism. The cutter spring 712 is depicted in a fully energized state in FIG. 44E, and a lever member 728 is positioned in an up position, ready to be pushed downward in response to manual actuation via the lever member 728. An indexer component 730 is engaged with a fixed ratchet member 732 that includes a plurality of notches 733, and the suture has been passed through the distal opening 734 of the shaft assembly. The lever member 728 may then be depressed downward, as shown in FIG. 44F. A cam slot 736 drives proximal movement of a movable ratchet member 738 having a plurality of notches 739, which is attached to the movable channel 704. Meanwhile, the indexer component remains 730 engaged with the fixed ratchet member 732, holding the first UE 702a in position as the suture is drawn proximally, between the distal leg members 703a, 703b. The snap arm member 724 pulls the cutter member 706 proximally, along with the channel 704, drawing the suture between the distal leg members 703a, 703b. The cutter spring 712 may remain energized.

The lever member 728 may then be fully depressed, as shown in FIG. 44G. At the bottom of the lever stroke, the snap arm member 724 disengages from the channel 704 and the ledge portion 726, releasing the cutter spring 712 and channel 704, and causing the cutter member 706 to move distally toward the suture seated in the UE 702a. The indexer component 730 remains engaged with the first, most proximal notch 733a of the fixed ratchet member 732, and engages with the next, more distal notch 739b of the movable ratchet member 738. As shown in FIG. 44H, the cutter member 706 may cut the suture, thereby ejecting the first UE 702a, when the lever member 728 remain in its fully depressed position. The cutter spring 712 drives the cutter member 706 distally with sufficient force to cut the suture after the snap arm member 724 is released from the ledge portion 726. After the first cutting cycle is complete, the cutter spring 712 may be released and the lever member 728 may return to its initial, up position. The movable ratchet member 738 may pull the indexer component 730 to the next, more distal notch 733b of the fixed ratchet member 730, which shifts all axially stored UEs distally, ready for the next deployment. The channel member 704 also returns to its initial, distal-most position (FIG. 44I).

The delivery device and associated mechanisms shown in FIGS. 44A-44I may enable a multi-fire delivery system, improve suture cut consistency, and eliminate the need for the currently implemented white line maneuver.

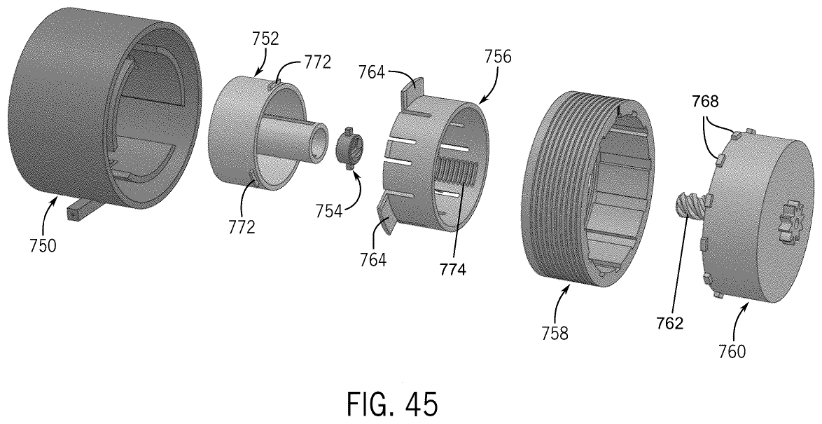

FIG. 45 provides an exploded view of the components of a delivery device configured to simultaneously hold and serially deploy multiple implants via a spool indexing mechanism. The components of the device may include a spool indexing assembly comprising a chassis 750, a shuttle member 752, a nut component 754, an indexer component 756, a spool 758 and a driver member 760 featuring an integral lead screw 762. As further shown, the chassis 750 may be fixed, the shuttle 752 may be configured for limited rotational translation, the nut component 754 may be configured to translate and rotate with the shuttle member 752, the indexer component 756 may translate, the spool 758 may rotate with the driver member 760 and translate with the indexer component 756, and the driver member 760 may rotate.

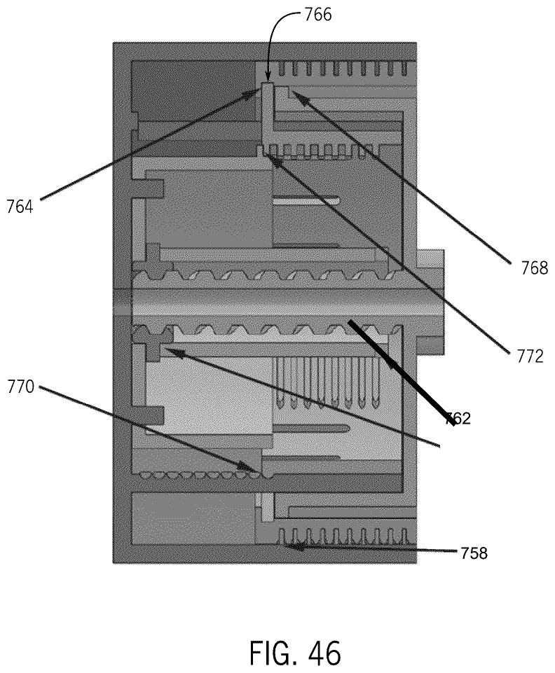

FIG. 46 shows the indexing spool components after assembly. As shown, tabs 764 on the indexer component may engage a complementary annular groove 766 in the spool 758. Splines 768 couple the driver member 760 and are configured to spool rotationally. One or more detents 770 are configured to prevent unwanted axial movement of the coupled indexer component and spool. One or more blade members 772 on the shuttle member 752 engage complementary ledge portions, notches, or a “ladder” portion 774 on the indexer component 756 to induce axial movement. The nut component 754 may be configured to travel the full length of the lead screw 762, while selectively moving the indexer component 756 at the far ends (distal and proximal).

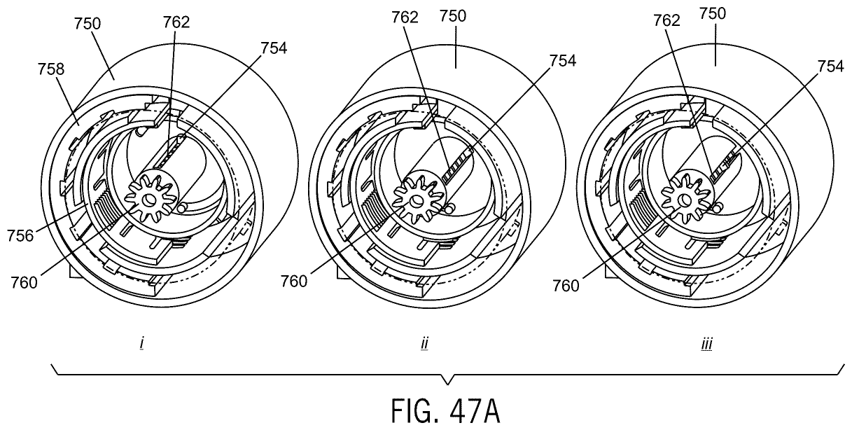

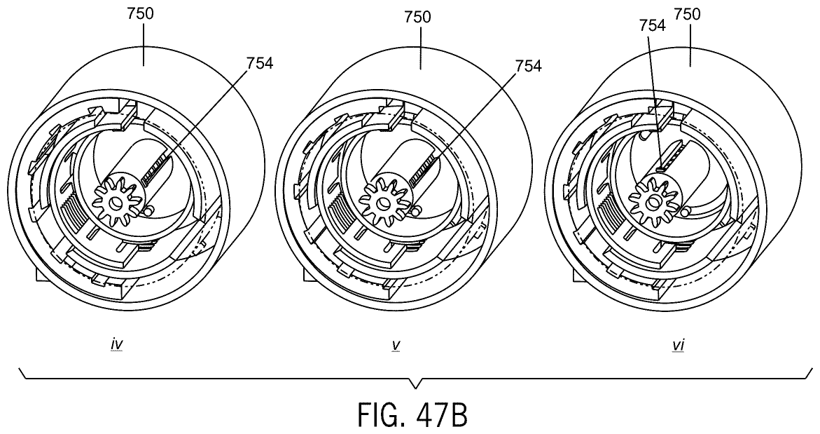

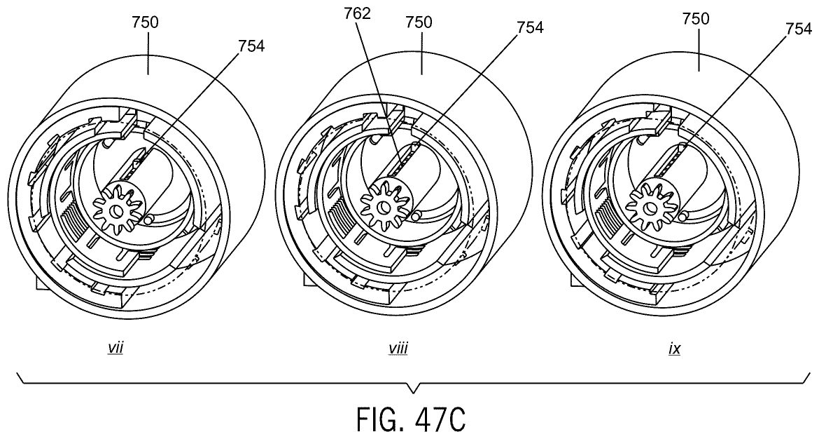

FIGS. 47A-47C illustrate snapshots of the spool indexing cycle (i-ix) implemented by the spool indexing assembly during activation of the delivery device to deploy CT/suture assemblies into a targeted lobe in serial fashion.

As shown in FIG. 47A, at the beginning of the cycle (i), the shuttle blades may remain engaged along the ladder portion of the indexer component at the same position as the end of the previous cycle. The driver member may be coupled to the spool via the spines, and the spool may be constrained to rotate with the driver, but also move axially.

For the first ˜60° of driver/spool rotation (ii), the nut component may turn with the driver member due to light friction between the complementary threaded portions, turning the shuttle member as well. The shuttle blades may disengage from the indexer ladder portion, and the spool may be coupled to the indexer component via the tabs in the annular groove, constrained to move axially only when induced to do so by the indexer component 756.

When the shuttle reaches its rotational limit (iii), the nut component 754 may be forced to translate along the lead screw 762, which may be an integral component of the driver. The shuttle member may or may not move axially during the illustrated phase, which may not impact the mechanism because the blades are disengaged from the indexer ladder at that point in the cycle.

As shown in FIG. 47B, the nut component 754 may continue to translate as the drum/lead screw turns (iv), while the shuttle member remains stationary. The nut wings make contact with the shuttle slot end at ˜850° of rotation.

The final ˜50° of driver/spool rotation (v) may continue to drive the nut component 754, which may now also move the shuttle member axially by ˜1.5 mm, which corresponds to the pitch of the suture spool. The suture payout may be complete at ˜900° of rotation (2.5 turns), corresponding to ˜15.5 inches of suture. The shuttle blades may bypass the indexer ladders due to earlier rotation of the shuttle and are now positioned to engage the next set of rungs in the indexer ladders.

After implant deployment (vi), the first ˜60° of driver/spool return rotation, the nut component 754 turns with the driver due to light friction between the threads, turning the shuttle with it. The shuttle blades may engage the next set of rungs in the indexer ladders, and the rotational limit stops the shuttle from turning when the blades have engaged the indexer ladder.

As shown in FIG. 47C, when the shuttle reaches its rotational limit (vii), the nut component 754 may be forced to translate along the lead screw 762, and the detents may prevent premature indexing of the spool while the nut component 754 is translating within the shuttle.

At ˜850° of driver/spool rotation (viii), the nut wings may contact the shuttle slot ends. From ˜850° to ˜900°, the spool may be translating. It may be critical that at this point, the suture remnant has been drawn entirely within the barrel to avoid jamming. This may correlate to a minimum length of 0.85 inches having been removed during implant deployment.

For the final ˜50° of driver/spool return rotation (ix), the nut component may drive the shuttle 1.5 mm axially, which also moves the indexer. With the spool coupled to the indexer, the spool may also move ˜1.5 mm, aligning the next suture with the drum opening. The indexing detent may be overcome by the axial force of the nut component. Suture retraction may be complete at ˜900° rotation (˜2.5 turns), and the cycle may be set to begin again.

The spool indexing mechanism shown in FIGS. 47A-47C may advantageously not require a manifold. All of the CT/suture assemblies may enter and exit through a single hole, which reduces the complexity of the overall design and may increase the support provided to the suture.

As described above, embodiments of the systems and devices described herein are configured to simultaneously hold and serially deploy multiple implants during a procedure without removing the elongate shaft assembly from a patient. Non-limiting examples of such devices deploy the implants to a treatment site, e.g., prostatic urethra, via manual engagement with one or more actuators, such as a trigger and/or lever member. Embodiments of the delivery device may be actuated multiple times to deploy each implant stored within the device. In the example described below, three actuations or trigger pulls of a first actuator, e.g., a trigger, followed by one actuation of a second actuator, e.g., a lever, are utilized to fully deploy an implant and reload or recharge the delivery device for the next implant deployment. Additional embodiments may require multiple actuations of only one actuator, e.g., trigger, such that, for instance, instead of three actuations of a first actuator (e.g., trigger) and one actuation of a second actuator (e.g., lever member), four actuations of the first actuator may be implemented without the involvement of a second actuator. Embodiments may also feature two or more actuators, each actuator configured to drive a portion of the implant deployment process, e.g., needle firing, implant assembly, suture cutting, etc., in response to manual engagement, e.g., squeezing, thumb press, etc. The terms “reload” and “recharge” may be used interchangeably below, referring to the restoration of the device (or components or subassemblies thereof) to a state ready to deploy a pre-loaded implant or execute one or more steps necessary to the deployment process, for example storing energy in one or more spring mechanisms.

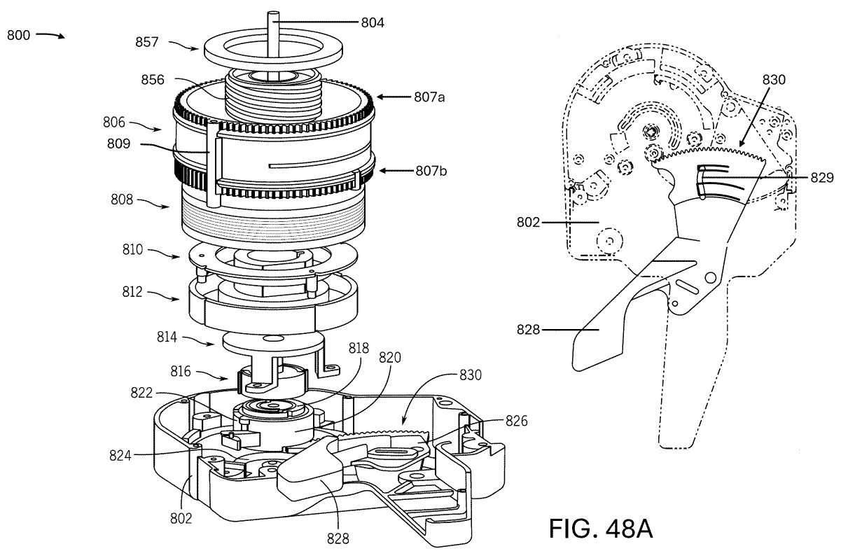

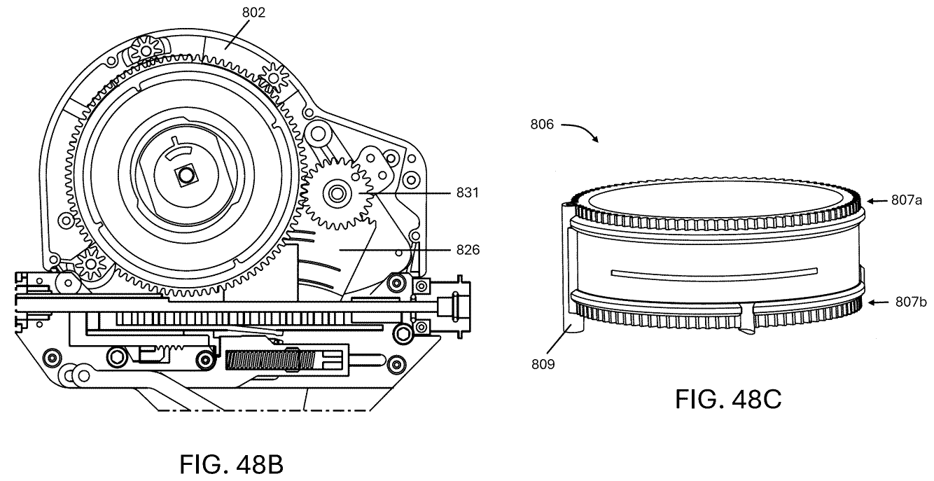

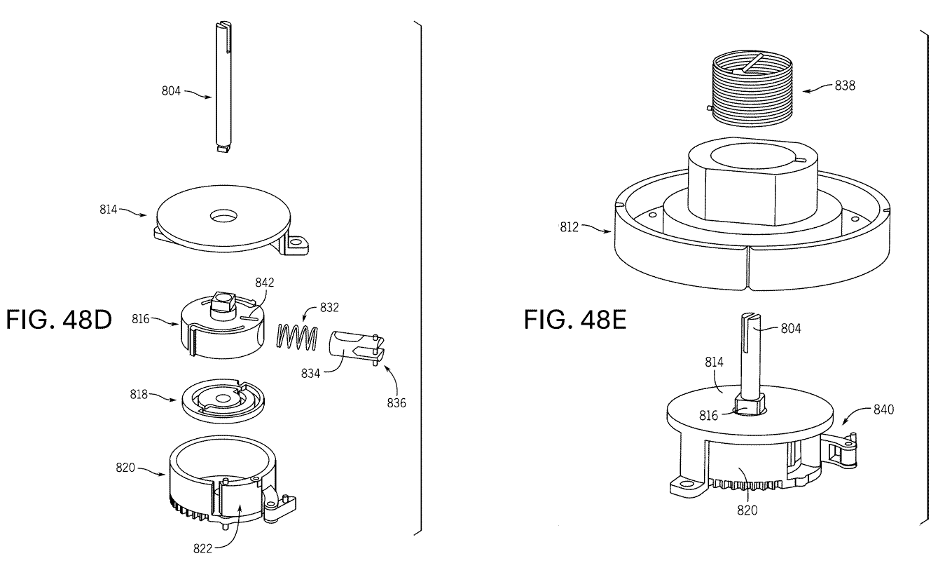

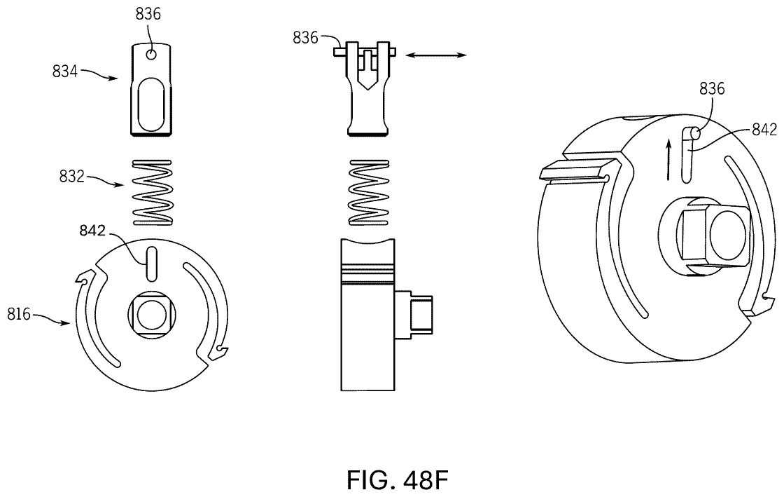

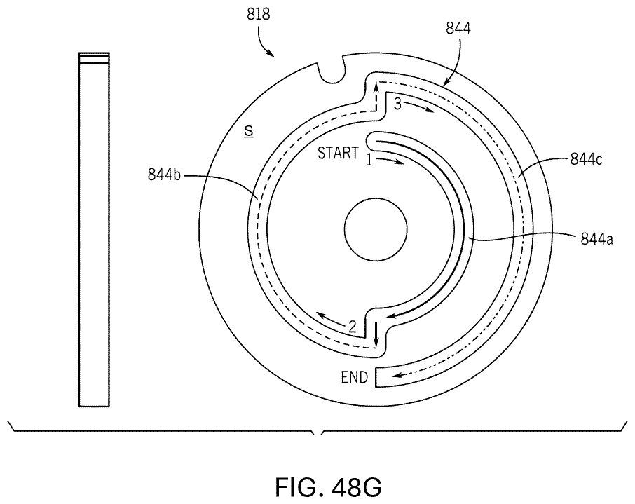

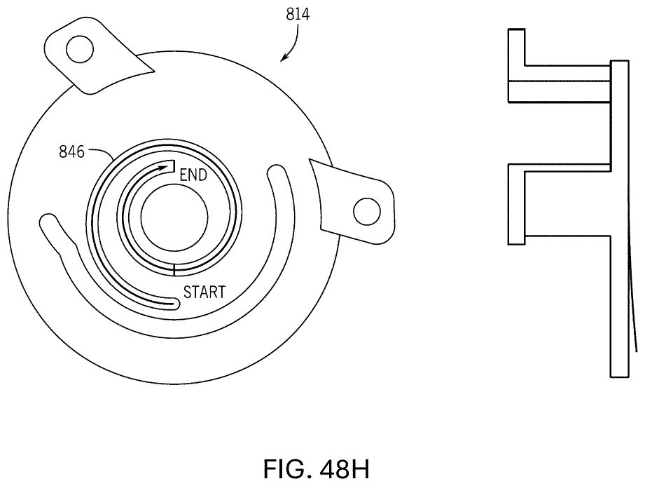

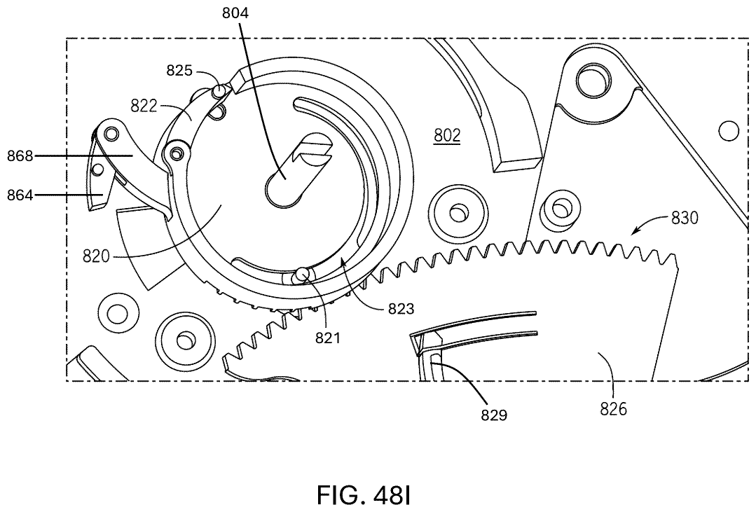

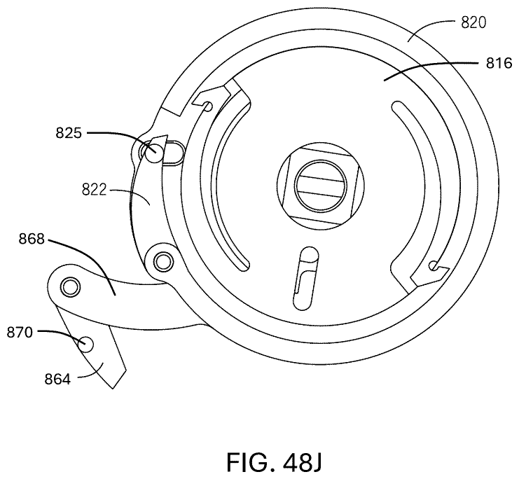

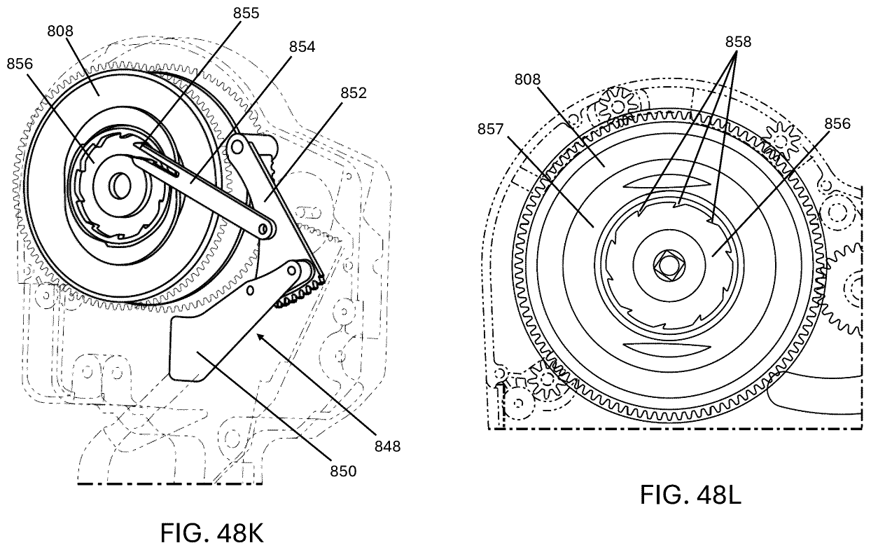

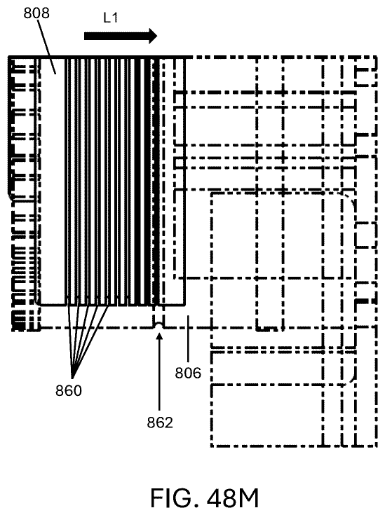

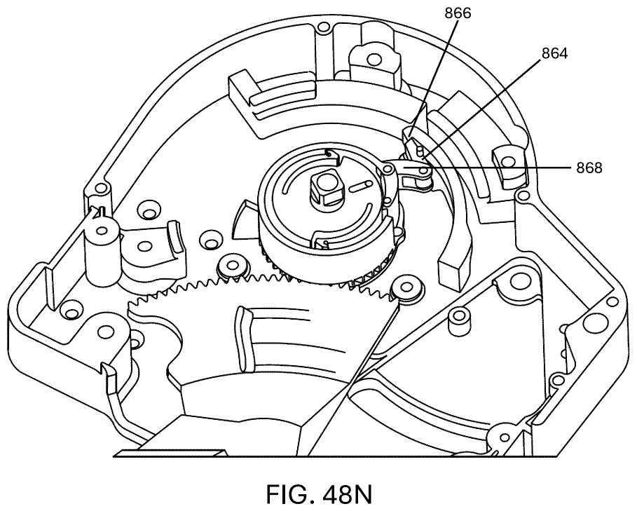

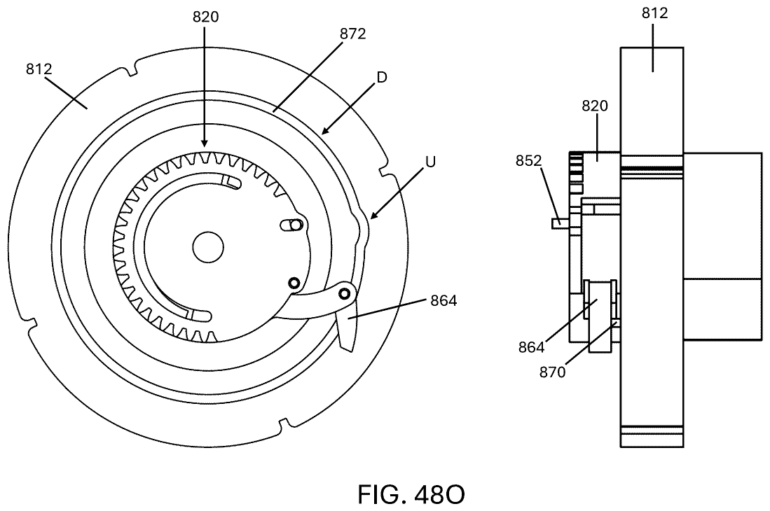

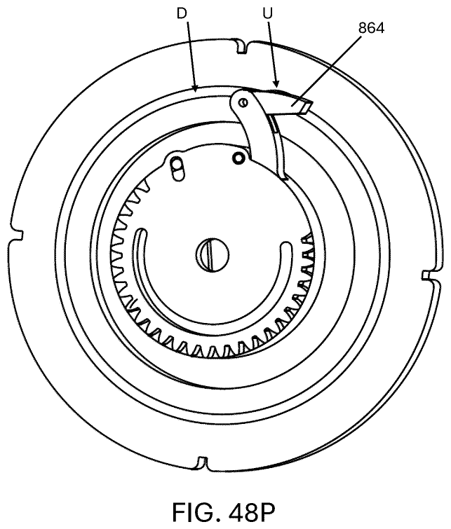

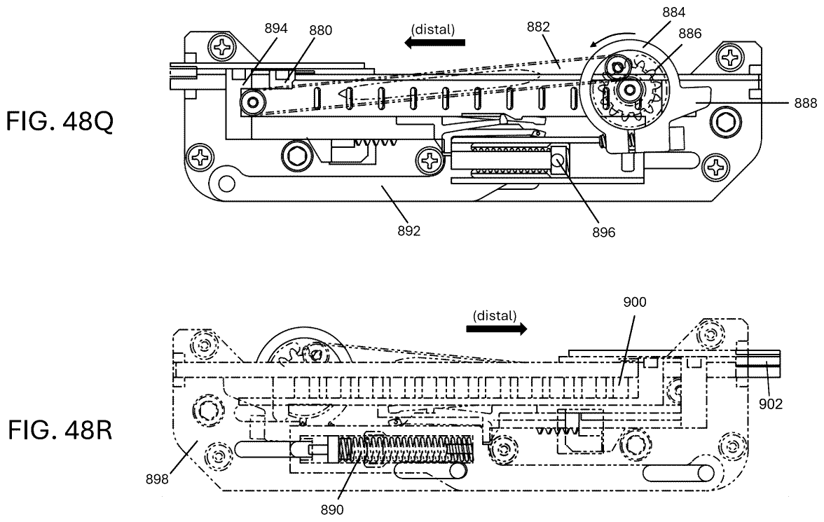

An example of an implant delivery device configured to drive a multi-fire implant deployment process via repeated manual engagement with at least one actuator is illustrated in FIGS. 48A-48R. Among other features, the components and subassemblies of the device may include one or more spools or spool members, e.g., two spool members, which may rotate at different speeds in response to trigger pull and release. One spool member (e.g., a first spool member) may be configured to hold and unwind the CT/suture assemblies, and one spool member (e.g., a second spool member) may be configured to unwind (deploy) and wind (retract) a delivery needle. A ratcheting mechanism in the handle assembly may control serial deployment of the pre-loaded CT/suture assemblies, and an indexing mechanism may control the serial deployment of multiple UEs. The device may be equipped with a CT pull-through recovery mechanism and associated bailout feature.

Embodiments of the implant delivery device may simultaneously hold eight or more implants, including nine, ten, 11, 12, 13, or 14 implants, or more. The device may also hold fewer than eight implants, e.g., two, three, four, five, six, or seven implants. In some examples, the delivery device may be loaded with a desired number of implants, which may be equal to or less than the maximum implant capacity of the device.

As set forth in greater detail below, examples of the device may include a first actuator, e.g., a trigger, and a method of deploying an implant may involve pulling and releasing the trigger two or more times, e.g., three times, four times, or more. The device may include more than one actuator in some examples. For instance, a second actuator may comprise a second trigger or a different type of actuator, such as a lever member, knob, switch, push-button, etc. The following example involves a delivery device that includes two actuators in the form of a trigger and a lever member. Multiple actuations of one or both actuators may be implemented to deploy each implant, including three pulls of the trigger and one actuation of the lever member.

The first trigger pull of the device may cause the elongate delivery needle of the device to fire, i.e., advance through the elongate shaft assembly until a distal end of the needle extends beyond a distal end of the shaft assembly. The first trigger pull may also charge a needle spring coupled to the needle, recharge a cutter spring coupled to a suture cutter member, load a tension spring to about one-third its capacity, advance a CT/suture assembly about one-third of the way toward the end of the shaft assembly, and charge a trigger return spring. The distal end of the needle may remain positioned beyond the distal end of the shaft assembly upon release of the trigger.

A second trigger pull may further load the tension spring to about two-thirds of its capacity, advance the CT/suture assembly to about two-thirds of the way toward the end of the shaft assembly, and charge the trigger return spring.

A third trigger pull may push the CT beyond the distal end of the deployed needle, and the needle may retract proximally, thereby unsheathing the CT and at least a portion of the suture attached thereto. The third trigger pull may also fully load the tension spring and charge the trigger return spring. Release of the third trigger pull also tensions the suture.

A fourth actuation may be accomplished via engagement with a lever member, which may be separate from the trigger. Actuation of the lever member may attach a UE to the tensioned suture and cut the suture just proximal to the UE. Release of the lever may index the next CT and UE within the device in preparation for subsequent deployment of the same.

The same process of sequential actuations may be repeated to deploy the next implant, which may occur after repositioning of the shaft assembly within the prostatic urethra such that the distal tip of the shaft assembly is located at the next targeted injection site.

One example of a delivery device configured to store and serially deploy multiple implants in this fashion is shown in FIG. 48A, which provides an exploded view of one embodiment of a handle assembly 800 (left) and a partially transparent side view of the handle case and trigger (right). Only one half or side of the housing or case 802 is shown to reveal the internal features of the handle assembly. Enclosed within the case 802 is an assembly of components together configured to deploy and reload the delivery device during the trigger-driven implant deployment process outlined above. The assembly includes a spindle 804, a needle spool member 806 defining two gearing portions 807a, 807b and an outer protrusion 809 or boss portion, a suture spool member 808, a suture spool post insert member 810, a suture spool guide member 812, a return plate 814, a spool driver member 816, a ratchet plate 818, a drive gear member 820, a driver pawl 822, and a cam path 824 defined by the case 802. An actuator in the form of a trigger 826 is also shown, with an external, manually engageable portion 828, an internal wall or raised portion 829, and an internal gearing portion 830. The wall portion 829 of the trigger 826 serves as a cam path for the needle spool member 806, contacting the outer protrusion 809 upon actuation of the trigger 826. Pulling of the trigger 826 causes both the suture spool member 808 and the needle spool member 806 to rotate, with the suture spool member 808 rotating at a faster rate than the needle spool member 806. Additional embodiments may combine, integrate, or omit one or more of the illustrated components. The elongate shaft assembly attached, coupled, or formed with the handle assembly is not shown for ease of illustration. Additional components of the delivery device, such as the delivery needle, are also not shown merely for ease of illustration, along with one or more implant components, e.g., CTs, sutures, and/or UEs. The devices, components, members, and assemblies shown in FIGS. 48A-48R may be compatible with one or more, including all, of these features omitted from illustration in FIGS. 48A-48R. The delivery needles shown and described herein, e.g., needle 162, 32, and/or 251, for instance, may be used with the embodiments shown and described in connection with FIGS. 48A-48R. The implants and components thereof shown and described herein, e.g., implant 10 and/or the implant and components shown in FIG. 4A, for instance, may also be used with the embodiments shown and described in connection with FIGS. 48A-48R.

FIGS. 48B and 48C show additional features of the handle assembly 800, including those specifically involved in actuation of the needle. A needle return gear member 831 configured to advance and retract the needle is shown in FIG. 48B. The needle return gear member 831 may be connected directly or indirectly to a torsion spring, one end of which may be fixed to the handle case 802. The needle return gear member 831 may also mesh or interact with the gearing portions 807a, 807b of the needle spool member 806, such that the needle spool member 806 and needle return gear member 831 rotate together. Rotation of the needle return gear member 831 may charge the torsion spring attached thereto, readying the device for spring-driven retraction of the needle following its advancement into the targeted tissue. In operation, the first trigger pull may cause rotation of the needle spool member 806 and deployment of the needle attached thereto. The first trigger pull may also charge the torsion spring directly or indirectly to the needle return gear 831, configuring the torsion spring to eventually retract the needle. The needle may remain stationary after the second trigger pull, for example via a braking mechanism, such that the distal end of the needle may remain positioned beyond the distal end of the shaft assembly. The third trigger pull releases the braking mechanism in the handle, which releases the needle spool member 806 and retracts the needle back into the shaft assembly. Embodiments of the braking mechanism may include temporarily abutting feature(s) between the needle spool member 806 and the handle case 802. For example, the handle case 802 may include one or more brake structures or stops arranged to contact and prevent counter-rotation of a corresponding ramped portion of the needle spool member 806.

FIGS. 48D and 48E highlight certain device components more directly involved in the ratcheted CT/suture deployment process, which may involve correlated actions of the suture spool member 808, the return plate 814, the spool driver member 816, the ratchet plate 818, a shuttle spring 832, a shuttle member 834, a shuttle pin 836, and a suture tension spring 838. These components may interact directly or indirectly to form a ratcheting mechanism 840 in which the suture spool guide member 812 rotates in a sequential, stepwise manner to advance each CT/suture assembly distally through the shaft assembly in a similarly stepwise manner.

In one non-limiting example, the suture spool guide member 812 may rotate a defined amount, e.g., 180 degrees or about 180 degrees, per trigger pull for three trigger pulls, which may sum to a defined total amount of rotation, e.g., 540 degrees or about 540 degrees of rotation, per implant deployment. After the first trigger pull, a CT/suture assembly wound around the suture spool member 808 may unravel from the suture spool member 808 and advance about one-third of the way to the distal end of the needle. After the second trigger pull, the CT/suture assembly may advance about two-thirds of the way to the distal end of the needle. After the third trigger pull, and thus the final partial rotation of the suture spool guide member 812, the CT/suture assembly may be fully advanced, such that the CT is anchored to the prostatic capsule, and the suture may be tensioned upon trigger release. After a fourth actuation, which may involve manual engagement with a separate lever member, a UE may be attached to the tensioned suture to form a complete implant. The suture may also be cut to fully deploy the implant, and the excess suture retracted back into the distal end of the shaft assembly.

FIG. 48F depicts two exploded views of certain CT/suture deployment components, including the spool driver member 816, shuttle spring 832, shuttle member 834, and shuttle pin 836 (left is plan view; middle is side view), along with one view of the components after coupling (right). The spool driver member 816 may house the shuttle member 834, shuttle pin 836, and shuttle spring 832. The shuttle spring 832 may be a compression spring positioned to apply a radially outward force to the shuttle member 834. The shuttle pin 836 may be mated in a hole defined by the shuttle member 834, the hole configured to allow the pin to move up and down (see double-sided arrow), i.e., bidirectionally along a path approximately perpendicular to the lateral movement of the shuttle member 834. The shuttle pin 836 may also limit movement of the shuttle member 834 within the slot 842 of the spool driver member 816. In operation, the shuttle member 834 may begin at a position near the radial center of the spool driver member 816, then move radially outward in stepwise fashion over the course of the triggering sequence to deploy a CT/suture assembly.

Positioning and movement of the shuttle pin 836 with respect to the ratchet plate 818 and return plate 814 upon each trigger pull is shown in FIGS. 48G and 48H, respectively. The ratchet plate 818 includes a groove or track 844 configured to receive and accommodate movement (e.g., sliding) of the shuttle pin 836. Before initiation of an implant deployment process, the shuttle pin 836 may begin (e.g., inserted, positioned, resting, or seated) at the starting position or first end (labeled “1”) of the first curved portion 844a of the track, which in this example of the ratchet plate 818 is the innermost track segment. Examples of the track may be referred to as a suture deployment track.

During the first trigger pull, the spool driver member 816 rotates, e.g., about 180 degrees, causing the shuttle pin 836 to travel along the first curved portion 844a of the track 844 (see solid arrow). At the end of the first trigger pull, the shuttle pin 836 is pushed radially outward to the first end (“2”) of the second curved portion 844b, which in this example is the middle track segment, where the back wall connecting the first curved portion 844a to the second curved portion 844b prevents the spool driver member from rotating backward, e.g., counterclockwise.

During the second trigger pull, the spool driver member 816 rotates again, e.g., another 180 degrees, causing the shuttle pin 836 to travel along the second curved portion 844b (see long-dashed arrow). At the end of the second trigger pull, the shuttle pin 836 may be pushed radially outward to the first end (“3”) of the third and final curved portion 844c, which in this example is the outermost segment of the track 844. The back wall connecting the second curved portion 844b to the third curved portion 844c may prevent the spool driver member from rotating backward, e.g., counterclockwise.

During the third trigger pull, the spool driver member 816 rotates again, e.g., another 180 degrees, causing the shuttle pin 836 to travel along the third curved portion 844c (see short-dashed arrow). Near the end of the third curved portion 844c, the bottom surface of the track 844 may begin to incline or ramp upward (i.e., away from planar surface S of the ratchet plate 818), pushing the shuttle pin 836 upward, away from the surface S of the ratchet plate 818, and switching its engagement from the ratchet plate 818 to the return plate 814. At the start of the release of the third trigger pull, the shuttle pin 836 may be engaged onto the outermost end or portion (“start”) of a track 846 on the return plate 814, shown in FIG. 48H. As the trigger is released and the suture ultimately cut after implant deployment, the tension spring 838 coupled directly or indirectly to the suture spool guide member 812 may cause rotation of the shuttle pin 836 back to the innermost end of the return plate track 846 (“end”). The end portion of the return plate track 846 may also have a ramped surface that pushes the shuttle pin 836 back down into engagement with the ratchet plate 818, thereby resetting the ratchetting mechanism for the next implant deployment.

The deployed suture may be tensioned by the torsion spring 838, the loading of which may also be linked to actuation of the trigger. The trigger 826 may actuate the suture spool member 808 through the drive gear member 820, guided by the cam path 824 in the handle case 802. The first trigger pull may load the tension spring 838 to about one-third its capacity, the second trigger pull may load the tension spring 838 to about two-thirds its capacity, and the third trigger pull may full load the tension spring 838. Anchoring of the CT to the prostatic capsule prevents the suture from retracting back into the shaft assembly upon loading of the torsion spring 838.

FIG. 48I provides another illustration of device components contributing to CT/suture deployment, highlighting the mechanical interactions between the drive gear member 820 and gearing portion 830 of the trigger 826, along with the components configured to constrain rotation of the drive gear member 820. Actuation of the trigger 826 may cause the drive gear member 820 to rotate about the spindle 804, which may be mated to the bottom handle case 802. In some examples, the drive gear member 820 may be limited to a defined rotation maximum, e.g., about 200 degrees, due to the stroke of the trigger 826 as well as a pin 821 protruding from the handle case 802 through an inner aperture or slot 823 in the drive gear member 820. When the trigger 826 is pulled, the drive gear member 820 rotates, e.g., clockwise. When the trigger 826 is released, the drive gear member 820 rotates back, e.g., counterclockwise, causing concomitant rotation of the drive gear member 820, limited by the interaction between the pin 821 and slot 823.

The driver pawl 822 may hinge directly into the drive gear member 820. The driver pawl 822 may include a driver pawl peg 825 that mates to the cam path 824 in the handle base cover 802. When the trigger 826 is positioned fully outward, i.e., after complete release, the driver pawl peg 825 may be positioned on an inclined ramp-up section of the cam path 824. When the trigger 826 is pulled, the drive gear member 820 rotates, e.g., clockwise, moving the driver pawl 822 down from the ramp-up section to the “down section.” When the trigger 826 is released, the driver pawl 822 rotates, e.g., counterclockwise, back up to the ramp-up section. The start and end position of the cam path 824 may correspond to the location of the driver pawl 822 at the start and end of the trigger pull, respectively, and vice-versa for trigger release.

FIG. 48J provides another illustration of the mechanical interaction between the drive gear member 820 and the spool driver member 816 in accordance with some examples. As shown, the drive gear member 820 and driver pawl 822 may cause rotation of the spool driver member 816, e.g., 180 degrees of rotation per trigger pull, as detailed above. As the trigger 826 is pulled, the drive gear member 820 rotates, e.g., clockwise. After approximately 20 degrees of rotation, the driver pawl 822 may move downward and contact a flexure arm of the spool drive member 816. Over the remaining 180 degrees of rotation, the driver pawl 822 may be rotating the spool driver member 820. At the end of the trigger pull, the spool member may remain stationary by the ratcheting mechanism 840, and the trigger 826 may release and rotate the drive gear member 820 back, e.g., counterclockwise. The driver pawl 822 may be free to flex the opposite flexure arm of the spool driver member 816 downward, out of the way. At the end of the third trigger pull, once the shuttle pin 836 has switched engagement from the ratchet plate 818 to the return plate 814, the spool driver member 820 may be free to rotate back (e.g., about 540 degrees) to its initial base position, ready for the next implant deployment.

FIGS. 48K and 48L illustrate components of the handle assembly involved in CT indexing. Embodiments may involve indexing successively deployed CT/suture assemblies via user engagement with an indexing lever 848 or other actuator directly or indirectly coupled to the suture spool member 808, which may move or shift laterally with each implant deployment. Examples of the indexing lever 848 may include a manually engageable portion 850 and an intermediary link member 852, the intermediary link member coupled with an indexing link member 854. A first, free end 855 of the indexing link member 854, which may include pronged portions, interacts with a rotatable indexing lead screw 856 coupled with an indexing nut 857 (see also FIG. 48A), the lead screw 856 defining or otherwise including a plurality of protrusions or teeth 858.

In operation, actuation of the lever 848 by a user via movement (e.g., thumb-press or depression, pulling, or pushing) of the engageable portion 850 moves the indexing link member 854 such that free end 855 reaches beyond the next tooth 858 of the indexing lead screw 856. Release of the lever 848 may then allow the free end 855 of the indexing link member 854 to grab or catch the adjacent tooth 858 and rotate the lead screw 856. Rotation of the lead screw 856 may cause lateral movement of the suture spool member 808, for example in the direction of the arrow L1 shown in the partially transparent side view of FIG. 48M, such that one of the CT/suture tracks 860 defined by the suture spool member 808 aligns with an opening or funnel 862 of the needle spool member 806. Rotation of the suture spool member 808 causes the CT/suture assembly aligned with the funnel 862 to unwind into the funnel 862 and feed into the needle attached (e.g., bonded) to the needle spool member 806, en route to the distal end of the shaft assembly of the delivery device. Each track 860 of a fully loaded device may contain one CT/suture assembly, which is constrained by the surrounding needle spool member 806 in a sleeve-like fashion to prevent unraveling of the CT/suture assemblies. Embodiments of CT/suture indexing may thus involve translation of a movable suture spool member relative to a stationary needle spool member in response to manual actuation, e.g., lever press. Movement of the suture spool member relative to the needle spool member may cause the CT/sutures to unwind from the suture spool member, one-by-one, and feed into the delivery needle coupled to the needle spool member.

The handle assembly 800 also includes a mechanism for recovering CT/suture assemblies and preventing device jamming after pull-through events. As shown in FIGS. 48N and 48O, the handle assembly 800 may include a recovery pawl 864 and a corresponding brake portion 866, the brake portion comprising a wall, ledge or protrusion that may be defined by or attached/fixed to the case 802. Together, the recovery pawl 864 and brake portion 866 may be configured to prevent retraction of a CT back into the shaft assembly in the event of a CT pull-through. The recovery pawl 864 may be pivotably coupled to an arm 868 of the drive gear member 820, which may be configured to move, e.g., pivot or swing, relative to the drive gear member 820. As shown in the side view of FIG. 48O (right), the recovery pawl 864 may include or be coupled with a peg member 870, which may have a square or approximately square body in some examples. The suture spool guide member 812 can be attached to the spool driver member 816 via the peg member 870, allowing the suture spool guide member 812 and spool driver member 816 to rotate together.

During the first trigger pull, the recovery pawl 864 may be positioned in a first, up position (“U”) defined by a track 872 of the suture spool guide member 812. The track 872 may be circular or approximately circular, and the position U may comprise or resemble a deviation, e.g., bulge, from the circular or approximately circular shape of the track. During the first trigger release, the suture spool guide member 812 may remain stationary while the drive gear member 820 rotates, moving the recovery pawl 864 from the first, up position U of the track 872 to a second, lower or down position (“D”) of the track 812, where the pawl 864 may bypass the brake portion 866 of the handle. As shown in FIG. 48O, the down position D may correspond to any position of the track, except for the portion of the track defining the up position U.

During the second trigger pull, the recovery pawl 864 may still be positioned in the down position D. The drive gear member 820 and suture spool guide member 812 may rotate together. During the second trigger release, the drive gear member 820 may rotate back, e.g., counterclockwise, while the suture spool guide member 812 remains stationary due to the CT ratcheting mechanism 840 described above. When in the down position D, the recovery pawl 864 again bypasses the brake portion 866.

During a normal release of the third trigger pull, i.e., a trigger release without a CT pull-through event, the drive gear member 820 and suture spool guide member 812 may rotate together, with the recovery pawl 864 in the up position U. The suture spool guide member 812 will stop rotating if a CT successfully anchors on the outer surface of a prostatic capsule, preventing the suture spool guide member 812 from rotating further. The drive gear member 820 may continue to rotate back, moving the recovery pawl 864 to the down position D, and again bypassing the brake portion 866 in the handle case 802.

In the event of a CT pull-through, the suture spool guide member 812 may not stop rotating after release of the third trigger pull. The drive gear member 820 and suture spool guide 812 will continue to rotate back together, with the recovery pawl 864 remaining in the up position U (see FIG. 48P), where it eventually contacts or catches the brake portion 866 in the handle case 802. This interaction may stall the drive gear 820 and prevent the unanchored CT from spooling back into the elongate shaft assembly of the delivery device. The normal out-sweep of the trigger may also stall, signaling to the user that a pull-through event has occurred and prompting the user to initiate a device bailout procedure, which may involve engaging an external component, e.g., key or key like feature, with the device.

FIGS. 48Q and 48R illustrate components of the handle assembly 800 involved in serial UE assembly and deployment implemented via a ratcheting index mechanism, with FIG. 48Q providing a front, partially transparent view and FIG. 48R providing a back, partially transparent view. Components include a movable ratchet member 880 or channel, a UE driver linkage 882, and a driver spur member 884, which includes or is coupled with a spur gearing portion 886 and a cutter spring release portion 888. Additional components include a cutter spring 890, a cutter spring energizer 892, a cutter anchor member 894, a push-button member 896, a fixed ratchet member 898, and a ratchet arm 900. The ratchet arm 900 includes a plurality of windows defined by vertical segments or rungs, the distance between each of which may be approximately equal to the length of a single UE. The UEs are aligned axially, end to end, within the shaft assembly 902 in this embodiment.

UE indexing and deployment may be initiated by causing the driver spur member 884 and its spur gearing portion 886 to rotate via actuation of lever member 848. Rotation of the spur gearing portion 886 urges (i.e., causes movement of) the UE driver linkage 882, and thus the movable ratchet member 880 attached thereto, forward in the distal direction (see left-pointing arrow in FIG. 48Q). Distal sliding of the movable ratchet member 880 may pull the UE ratchet arm 900 distally (right arrow in FIG. 48R), positioning the next UE in the deployment position at the distal end of the shaft assembly. Together, these actions index the UEs distally within the elongate shaft assembly and pull the suture into the distal-most UE, seating the UE onto the suture.

The cutter spring energizer 892, which may be operatively coupled to the trigger 826, may hold the cutter spring 890 in a loaded, compressed configuration. Continued rotation of the driver spur member 884 may eventually cause the cutter spring release portion 888 to contact and depress the push-button member 896, disengaging the cutter spring energizer 892 from the cutter spring 890. The released cutter spring 890 fires forward (distally), pushing the cutter member within the shaft assembly into the tensioned suture and cutting the suture just proximal to the seated UE. Configurations of the push-button member 896 may vary and may include members, elements, components, and/or portions of the device that do not resemble a push-button, including any biased or movable component (e.g., switch, knob, slide, and/or sled), which may assume different configurations in response to contact or force applied by another component of the device.

Accordingly, embodiments of a multi-fire delivery device (e.g., device 800) may include a first actuator (e.g., trigger 826) and a second actuator (e.g., lever member 848). Actuation of the first actuator may drive deployment of each CT/suture, and actuation of the second actuator may index and/or deploy the urethral endpieces, which may be aligned end-to-end in the shaft assembly. Embodiments may include two or more spool members, including for instance a first spool member (e.g., suture spool member 808) and a second spool member (e.g., needle spool member 806). The first spool member and the second spool member may rotate at different speeds, for example in response to actuation of one or more actuators (e.g., trigger 826). In some examples, the first spool member may be configured to move relative to the second spool member. The first spool member (e.g., suture spool member 808) may define a plurality of circumferential tracks (e.g., tracks 860), each track configured to accommodate one CT/suture. In some examples, movement of the first spool member relative to the second spool member causes the CT/sutures to unwind from the first spool member and feed into the delivery needle via an opening (e.g., funnel 862) defined by the second spool member. The actuator(s) included in some examples may cause serial deployment of the implant assemblies via a ratcheting mechanism (e.g., ratcheting mechanism 840). In some examples, the ratcheting mechanism may include a ratchet plate defining a track (e.g., track 844), which may be considered a suture deployment and/or tensioning track. Methods for compressing prostate tissue, for example as a means of treating benign prostatic hyperplasia, may involve advancing an elongate shaft assembly of a delivery device (e.g., device 800) through a urethra until a distal end of the elongate shaft assembly is positioned adjacent to the prostate tissue, where the delivery device further includes a handle assembly attached to a proximal end of the elongate shaft assembly. The handle assembly may include a spooling assembly comprising a first spool member (e.g., suture spool member 808) and a second spool member (e.g., needle spool member 806), along with one or more manually engageable actuators configured to cause deployment of the implant assemblies in serial fashion. Methods may involve deploying the prostatic implant assemblies into the prostate tissue by actuating at least one of the manually engageable actuators (e.g., trigger 826) two or more times (e.g., three times or four times, via trigger pull).

In some embodiments, an implant delivery system and one or more associated method steps involved in deploying one or more implants disclosed herein may be automated in whole or in part. Embodiments of an automated system may include one or more electromechanical components configured to deploy one or more implants in response to receipt of a manual input, e.g., via button push. Embodiments of an automated system may additionally or alternatively be battery operated. Examples of one or more automated systems may lack one or more hardware components disclosed in other embodiments described herein, such as one or more device actuators, non-limiting examples of which may include a trigger assembly or lever member. One or more motorized components may be included.

In some examples, one or more steps of a disclosed implant deployment process may be automated. For instance, distal firing of a needle through an elongate shaft assembly may be automated. Retraction of the needle may also be automated, together with distal advancement and/or unsheathing of a CT/suture assembly. In some examples, suture tensioning, UE attachment, and/or suture cutting may be automated, as may be resetting of a delivery device for the deployment of a next CT/suture. Implants or portions thereof, e.g., CT/suture assemblies, may be indexed in automated fashion, for example via a motorized cam system.

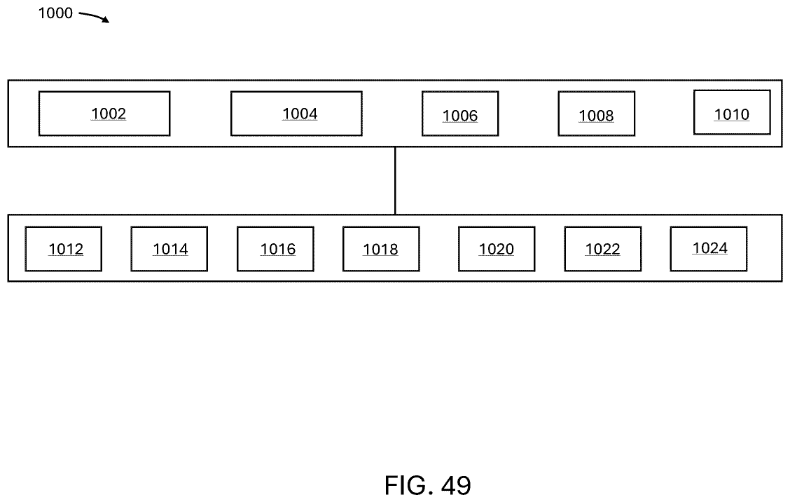

Examples of automated system features and components 1000 are represented schematically in FIG. 49. An automated system 1000 may include an electronic core unit 1002 comprising or communicatively coupled with a control unit 1004, which may comprise embedded software or PCB incorporated or coupled with an electronic handheld delivery device 1006, which may include a handle assembly and elongate shaft assembly loaded with multiple implant assemblies or configured to receive removable cartridges, as described herein. Examples of an automated system may include a combination of reusable components 1008 and disposable components 1010, the latter of which may include a shaft assembly, delivery needle, chip-on-tip visualization component(s), CT/suture assemblies, UEs, and/or actuators configured to transfer rotational energy from a motorized component, and a disposable case. In some examples, a reusable unit 1008 may include a battery, electronic and motor unit system, which may be coupled with an upper disposable unit containing one or more needles and/or implants. One or more additional disposable elements may also be included, non-limiting examples of which may include one or more components of an expandable wing member or assembly, for example as described in U.S. Pat. No. 11,672,520, the entire contents of which are incorporated by reference herein. Embodiments featuring one or more reusable components may feature an electronic circuit board, electrical system, motor, battery pack, switch, LED indicators, and an LCD screen. Various embodiments, including those coupled with disposable and non-disposable devices and components thereof, may include one or more batteries 1012, a motorized cam system 1014 to operate CT and UE indexing, one or more positioning sensors 1016 to detect CT and UE locations, one or more force sensors 1018 to inform suture tensioning, a needle ejection controller 1020 configured to vary needle speed during distal advancement and proximal retraction, one or more displays 1022 (e.g., LED/LCD indicators for the number of implants completed and other sensing information), and/or a chip-on-tip system 1024 configured to visualize and provide real-time guidance for implant placement.

Embodiments of an automated system 1000 may also include one or more sensors configured facilitate, monitor, and/or in some examples control one or more aspects of an implant delivery process. For example, a sensor configured to measure the compression force applied to targeted prostatic tissue may be included. Associated methods of implant deployment may involve adjusting one or more configurations of the implant(s) and/or settings of the delivery device to apply the appropriate amount of compression and/or tension to the targeted tissue, thereby ensuring sufficient tissue retraction while minimizing the likelihood of CT pull-through events. Accordingly, the sensors may provide measurements and information utilized in adjusting the delivery device configuration and/or positioning. Adjustments may be performed by a user or via automatic adjustments implemented by the automated system trained to adjust implant deployment parameters in response to live feedback data obtained by one or more sensors.

Embodiments of an automated system may also include one or more components, e.g., force sensor(s), configured to measure suture tension to ensure appropriate tensioning based on linear measurements. Such embodiments may also be configured to adjust suture tensioning in automated fashion, based on the force measurements. A centralized electronic control unit may be incorporated to actively manage the applied force and resulting compression of the targeted prostatic tissue. Embodiments may include an encoder, (which may be mounted on a motor) or an optical sensor (configured to capture the suture/needle location) configured to capture linear movement.

The LED/LCD display(s) may be configured to provide a variety of information that may assist a user performing an implant procedure. For instance, the LED/LCD display(s) may be configured to display the number and/or location of implants currently implanted, as well as whether a procedure has been completed or successfully performed based on the number and/or position of deployed implants, which may be provided alone or in combination with measured compression forces. A display unit may thus provide implant count, implant location, and/or force measurements acquired during a procedure to enable automated or user-controlled adjustments on the fly.

In some examples, a chip-on-tip component or assembly may be included for enhanced visualization of a treatment site within a prostatic urethra. Embodiments may include a communicatively coupled AI measurement system (e.g., motion capture sensor used with image analysis) together with an LCD display. Such implementations may be configured to show the distance traveled of the distal end of the shaft assembly and/or its distance from a target site in the prostatic urethra.

Embodiments of an automated system may also be equipped with a speed control feature, which may include a motor and associated control unit. Such embodiments may be utilized to control and customize the speed at which a needle punctures through the targeted prostatic tissue. In some examples, a speed control feature may control the needle such that it travels at a relatively high speed through the urethral side of the prostate, then reduces to a lower speed as the distal tip reaches the outer prostatic capsule, thereby reducing the likelihood of needle over-extension that could otherwise lead to bone strikes, which may damage the needle and prevent successful implant deployment, in addition to causing patient discomfort.

Embodiments may also include a force sensor, alone or with additional/alternative components configured to determine whether a distal end of the needle is contacting bone. Detection of a bone strike may prompt the system to generate an alert displayed to the user on the LED/LCD screen. The user may then re-position the shaft assembly as necessary to avoid continued bone strike. Various alerts and displays may be provided and updated in real time to assist the user as the procedure is being performed.

Examples of the automated system or component(s) thereof, represented in FIG. 49, may be included in one or more of the devices shown and described herein, for example one or more devices having or compatible with a removable cartridge, one or more devices configured to simultaneously hold and serially deploy multiple implants, one or more devices configured to deploy and/or index one or more UEs, and/or one or more devices configured to prevent damage caused by pull-through events, and/or one or more devices featuring one or more combinations of the aforementioned components and features. Accordingly, automated system components may be featured in one or more devices configured to deploy multiple prostatic implants during a single procedure, which may occur without removing the delivery device or component thereof, e.g., the shaft assembly, from the patient. Automated embodiments may be paired with one or more embodiments featuring a removable cartridge, spooling mechanism, implant magazine, or auto-indexing mechanism, for instance. Automated embodiments may also be paired with one or more embodiments featuring a UE deployment mechanism featuring, among other things, a stationary UE seating/cutting configuration and/or auto-indexing UE.

Automated components may also be included in a single-fire delivery device, which may be a device loaded with a single implant. The shaft assembly of such single-fire devices may require removal during a procedure to accommodate the exchange of a removable cartridge with the corresponding handle assembly or to accommodate the insertion into a patient of an entirely separate device (which may lack a removable cartridge) containing another implant. Non-limiting examples of such devices are described in U.S. Pat. No. 11,298,115 and U.S. Patent Application Publication No. 2021/0378658, the entire contents of which are incorporated by reference herein. Accordingly, the automated system components depicted in FIG. 49 may be incorporated into a variety of implant delivery devices, non-limiting examples of which may include, but are not limited to, the devices, device components, and device assemblies described herein.

### EXAMPLES

The above Detailed Description includes references to the accompanying drawings, which form a part of the Detailed Description. The Detailed Description should be read with reference to the drawings. The drawings show, by way of illustration, specific embodiments in which the present accessory devices and associated methods can be practiced. These embodiments are also referred to herein as “examples.”

The Detailed Description is intended to be illustrative and not restrictive. For example, the above-described examples (or one or more features or components thereof) may be used in combination with each other. For instance, one or more of the examples described herein for deploying multiple implants during a procedure, for instance using one or more spooling assemblies, indexing components, ratcheting components, and/or removable cartridges, may be paired with one or more examples described herein for deploying a UE and cutting a suture. One or more individual components of devices described herein, such as a spool member or actuator, may also be included in different devices described herein. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment.

From the foregoing it will be appreciated that, although specific embodiments of the invention have been described herein for purposes of illustration, various modifications may be made without deviating from the spirit and scope of the invention.

The disclosed devices, systems, assemblies, and components thereof can be implemented in various treatment devices employed for various medical purposes including, but not limited to, retracting lifting, compressing, approximating, supporting, remodeling, repositioning, ablating, or otherwise altering tissues, organs, anatomical structures, grafts, or other material found within the body of a human or animal subject. In certain embodiments, treatment devices are intended to displace, compress, retract, or destroy tissue of the prostate to facilitate treatment of disease or disorders, such as BPH.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

All relative, directional, and ordinal references (including top, bottom, side, front, rear, first, second, third, and so forth) are given by way of example to aid the reader's understanding of the examples described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other, unless specifically set forth in the claims.

The following claim examples are hereby incorporated into the Detailed Description, with each example standing on its own as a separate embodiment:

In Example 1, a delivery device configured to deploy multiple implants to a prostate gland of a patient without removing an elongate delivery shaft of the delivery device from the patient may include a handle assembly and a removable cartridge. The removable cartridge may be configured to contain at least one distal anchor component and a suture attached thereto (“CT/suture”), the distal anchor component configured to anchor to a prostatic capsule of the prostate gland, and the suture configured to be placed within the prostate gland. The handle assembly may include at least one actuator, a needle configured to receive the CT/suture and pierce through the prostate gland, and the elongate delivery shaft may be configured to be advanced longitudinally through the urethra of the patient while accommodating advancement of the needle therethrough. The removable cartridge may be removed from the handle assembly after insertion of the elongate delivery shaft within the urethra without removing the elongate delivery shaft from the urethra.

In Example 2, the delivery device of Example 1 may further include at least one spooling mechanism.

In Example 3, the spooling mechanism of Example 2 may include at least one spool member in the handle assembly and at least one spool member in the removable cartridge.

In Example 4, the at least one spool member in the handle assembly of Example 3 may include a needle spool configured to couple with the needle.

In Example 5, the at least one spool member in the removable cartridge of Example 3 may include a suture spool configured to couple with the suture.

In Example 6, the spooling mechanism of Example 5 may include one or more gear portions, features, members or surfaces configured to translate activation of the at least one actuator into rotation of the needle spool and the suture spool, causing deployment of the needle and the CT/suture positioned therein.

In Example 7, the at least one actuator of any one or any combination of Examples 1-6 may include a needle actuator and a suture actuator.

In Example 8, the needle actuator and the suture actuator of Example 7 may each be attached to a separate spring.

In Example 9, the needle actuator of Example 7 and/or 8 may include gear teeth configured to engage complementary gear teeth included on the needle spool.

In Example 10, the delivery device of any one or any combination of Examples 7-9 may further include a movable lever configured to hold the needle actuator in an energized state until released via manual engagement.

In Example 11, disengagement of the lever of Example 10 with the needle actuator may cause the needle to unwind from the needle spool and advance distally through the elongate delivery shaft, toward and through the prostate gland positioned adjacent to the delivery shaft.

In Example 12, the suture actuator of any one or any combination of Examples 7-11 may include gear teeth configured to engage the suture spool, such that activation of the suture actuator drives rotation of the suture spool, thereby unwinding the suture coupled with the suture spool.

In Example 13, the suture of any one or any combination of Examples 7-12 may be advanced through the needle and the prostate gland upon activation of the suture actuator.

In Example 14, the at least one actuator of any one or any combination of Examples 1-13 may include gear teeth and a raised rail portion.

In Example 15, the handle assembly of Example 14 may further include a needle spool member configured to couple with the needle, and the needle spool member may lack gear teeth.

In Example 16, the needle spool member of Example 15 may further include a post portion and a tab member. The tab member may be attached to a spring, and the post portion may be configured to contact and be moved by the raised rail portion of the actuator in response to movement of the actuator, which may cause the needle to unwind from the needle spool member and advance distally through the elongate delivery shaft.

In Example 17, the post portion of Example 16 may slide along the raised rail portion as the actuator continues to move until reaching an end of the raised rail portion, at which point the post portion may disengage the raised rail portion and retract the needle.

In Example 18, movement of the actuator of Example 16 or 17 may advance the CT/suture distally through the needle.

In Example 19, a delivery device configured to deploy multiple implants to a prostate gland of a patient without removing an elongate delivery shaft of the delivery device from the patient may include a handle assembly and a removable cartridge. The removable cartridge may be configured to contain at least one distal anchor component and a suture attached thereto (“CT/suture”), along with a needle configured to receive the CT/suture and pierce through the prostate gland. The distal anchor component may be configured to anchor to a prostatic capsule of the prostate gland, and the suture may be configured to be placed within the prostate gland. The handle assembly may include an actuator. The elongate delivery shaft may be configured to be advanced longitudinally through the urethra of the patient while accommodating advancement of the needle therethrough. The removable cartridge may be removed from the handle assembly after insertion of the elongate delivery shaft within the urethra of a patient without removing the elongate delivery shaft from the urethra.

In Example 20, the removable cartridge of Example 19 may be configured to be installed in the side of the handle assembly

In Example 21, the removable cartridge of Example 19 may be configured to be installed in the rear of the assembly.

In Example 22, the delivery device of any one or any combination of Examples 19-21 may further include at least one spooling mechanism configured to advance the needle and the CT/suture through the prostate gland in response to manual activation of the actuator.

In Example 23, the removable cartridge of any one or any combination of Examples 19-22 may further include a first gear member and a second gear member, the gear members rotatably coupled.

In Example 24, the first gear member of Example 23 may be configured to engage with a locking member, e.g., knob, on the cartridge during cartridge installation, such that locking the cartridge to the handle assembly via manual engagement with the locking member, e.g., rotation of the knob, may drive rotation of a needle spool member and advancement of the needle through the elongate delivery shaft.

In Example 25, a delivery device configured to deploy multiple implants to a prostate gland of a patient without removing an elongate delivery shaft of the delivery device from the patient may include a handle assembly. The handle assembly may include a first spool member configured to couple with the multiple implant assemblies simultaneously, each implant assembly comprising a distal anchor component (“CT”) and a suture attached thereto (“CT/suture”), the distal anchor component configured to anchor to a prostatic capsule of the prostate gland, and the suture configured to be placed within the prostate gland. The handle assembly may also include a manifold member coupled with the first spool member. The manifold member may be configured to receive the implant assemblies in a serial, controlled fashion.

In Example 26, the delivery device of Example 25 may further include a second spool member configured to couple with a needle configured to receive each CT/suture in the serial fashion and pierce through the prostate gland.

In Example 27, the delivery device of Example 26 may further include a plurality of pivotable, rotatable, hinged, or otherwise movable components, e.g., flexure plugs, attached to the second spool member and biased toward a closed position configured to prevent the CT/suture assemblies from unwinding from the first spool member and advancing through the manifold, into the needle.

In Example 28, the delivery device of Example 27 may further include a rotatable cam indexer configured to engage the movable components (e.g., flexure plugs) in the serial fashion, such that rotation of the cam indexer causes the movable components to transition to an open position in the serial fashion, thereby allowing passage of the CT/suture assemblies into the needle one-by-one.

In Example 29, the delivery device of any one or any combination of Examples 25-29 may further include an actuator attached to a spring and operatively coupled to the first and second spool members via gearing.

In Example 30, a delivery device configured to deploy multiple implants to a prostate gland of a patient without removing an elongate delivery shaft of the delivery device from the patient may include a spool member configured to couple with multiple implant assemblies simultaneously, each implant assembly comprising a distal anchor component (“CT”) and a suture attached thereto (“CT/suture”), the distal anchor component configured to anchor to a prostatic capsule of the prostate gland, and the suture configured to be placed within the prostate gland. The spool member may include multiple independently rotatable spools or spool portions, each rotatable spool configured to accommodate a CT/suture assembly. The device may further include a cam indexer configured to couple with the spool member and cause one-by-one rotation of each rotatable spool, as the other spools remain stationary, thereby advancing each CT/suture through a needle positioned within the elongate delivery shaft in serial fashion.

In Example 31, a delivery device configured to deploy multiple implants to a prostate gland of a patient without removing an elongate delivery shaft of the delivery device from the patient may include a magazine assembly configured to hold multiple distal anchor components (e.g., CTs) of the implants simultaneously, each distal anchor component attached to a suture. The delivery device may also include a needle assembly attached to the magazine assembly. The delivery device may also include a handle assembly, the handle assembly comprising an actuator and an elongate push member. The push member may be configured to be advanced longitudinally through the needle assembly in response to activation of the actuator.

In Example 32, the magazine assembly of Example 31 may include a spring-loaded element, e.g., plunger element, configured to urge each distal anchor component, one-by-one, into longitudinal alignment with the needle assembly, thereby indexing the implants during serial deployment thereof.

In Example 33, the elongate push member of Example 31 and/or 32 may be configured to push each distal anchor component, one-by-one, through the needle assembly.

In Example 34, the needle assembly of any one or any combination of Examples 31-33 may include a first needle portion and a second needle portion, with the magazine assembly positioned therebetween.

In Example 35, each of the implants of any one or any combination of Examples 31-34 may further include an enlarged portion at a proximal end of the suture, the enlarged portion configured to be engaged and pulled proximally after implant deployment, thereby tensioning the suture attached thereto and withdrawing surplus suture after suture cutting.

In Example 36, the needle assembly and magazine assembly of any one or any combination of Examples 31-35 may move together, and the elongate push member may move independently in response to activation of the actuator.

In Example 37, a delivery device configured to deploy multiple implants to a prostate gland of a patient without removing an elongate delivery shaft of the delivery device from the patient may include a handle assembly and a removable cartridge. The removable cartridge may include a spooling assembly configured to recover a distal anchor component (e.g., CT) and a suture attached thereto after the distal anchor component and suture tear through the prostate gland in the proximal direction after implantation.

In Example 38, the spooling assembly of Example 37 may include multiple spools, non-limiting examples of which may include a tension spool, a suture spool, a needle spool, and/or a caliper spool.

In Example 39, the handle assembly of Example 38 may include one or more spools, such as the needle spool and the caliper spool.

In Example 40, the removable cartridge of Example 39 may include one or more spools, such as the tension spool and the suture spool.

In Example 41, the device of any one or any combination of Examples 38-40 may further include a tension spring configured to tie rotation of the tension spool with the suture spool. The tension spool may be driven by an actuator on the handle assembly.

In Example 42, the tension spool of Example 41 may define a hard stop portion and ramped portion, and the caliper spool may define a one-way flexure.

In Example 43, the one-way flexure of Example 42 may be configured to be flexed to a non-obstructing configuration when the needle spool is rotated to fire a needle distally through the elongate delivery shaft.

In Example 44, the one-way flexure of Example 43 may be configured to extend back through a slot defined by the needle spool after the needle is retracted.

In Example 45, in the event of a pull-through, the one-way flexure of Example 44 may prevent the tension spool from rotating.

In Example 46, a delivery device configured to deploy multiple implants to a prostate gland of a patient without removing an elongate delivery shaft of the delivery device from the patient may include a handle assembly comprising the elongate delivery shaft. The device may further include a removable cartridge, which may be configured to contain at least one distal anchor component and a suture attached thereto (“CT/suture”), the distal anchor component configured to anchor to a prostatic capsule of the prostate gland, and the suture configured to be placed within the prostate gland. The elongate delivery shaft may include a puller member configured to pull the suture in the proximal direction, and a cutting element configured to cut the suture after attaching a proximal anchor component to the suture. The proximal anchor may remain stationary or substantially stationary during suture attachment and cutting.

In Example 47, the puller member of Example 46 may include two loop members each configured to pull the suture in the proximal direction.

In Example 48, the cutting element of Example 47 may include a blade positioned between the loop members.

In Example 49, pulling the suture of Example 48 proximally may cause the suture to attach to the proximal anchor component. Continued proximal pulling may cause the suture to be cut by the blade.

In Example 50, the puller member of any one or any combination of Examples 46-49 may further include an enclosure configured to house the suture until the suture is cut, upon additional retracting the puller member in the proximal direction.

In Example 51, a method for deploying one or more implants to a prostate gland of a patient may utilize the delivery device of any one or any combination of Examples 1-50.

In Example 52, the method of Example 51 may involve deploying two or more implants, and a shaft assembly of the delivery device may not require removal from the patient until all of the implants necessary to complete a procedure are deployed.

In Example 53, a method for treating benign prostatic hyperplasia may be performed using the delivery device of any one or any combination of Examples 1-50.

In Example 54, means for compressing a prostate gland of a patient may utilize the delivery device of any one or any combination of Examples 1-50.

In Example 55, means for compressing an enlarged prostate gland of a patient may utilize the delivery device of any one or any combination of Examples 1-50.

In Example 56, means for treating benign prostatic hyperplasia may utilize the delivery device of any one or any combination of Examples 1-50.

In Example 57, means for deploying one or more prostatic implants to a prostate gland utilize prostatic implants configured to compress or retract at least a portion of the prostate gland.

Example 58 includes means for forming a delivery device, system, assembly, and/or component of any one or any combination of Examples 1-50 by molding, injection molding, metal working, metal stamping, and/or welding, along with the attachment, coupling, and/or fixing of the components formed via such processes.

In Example 59, a method for deploying one or more implants to one or more lobes of a prostate gland may involve one or more of the following steps: 1) positioning an elongate portion (e.g., shaft assembly) of a delivery device disclosed herein in a prostatic urethra; 2) advancing a penetrating member or needle coupled with at least a portion of a prostatic implant through the elongate portion of the delivery device, the prostatic implant comprising a distal anchor portion (e.g., CT) connected to a middle portion (e.g., suture); 3) penetrating a lobe of the prostate gland with the distal tip of the needle; 4) advancing the needle through the lobe of the prostate gland until the distal tip is positioned outside a prostatic capsule of the lobe; 5) unsheathing the prostatic implant by retracting the needle in a proximal direction; 6) tensioning the middle portion; and 7) securing a proximal anchor to the middle portion.

In Example 60, the method of Example 59 further involves repositioning the elongate portion of the delivery device in the prostatic urethra and repeating one or more of steps 2-7 to deploy a second implant in the prostate gland.

In Example 61, the elongate portion of the delivery device of Example 60 may remain within the urethra, such that the elongate portion is not removed from the patient between deployment of the first and second implants.

In Example 62, the delivery device of Example 61 may include a handle assembly, and each of the one or more implants may be included in a separate removable cartridge configured to be coupled with the handle assembly, such that deploying the second implant involves exchanging a first removable cartridge initially containing the first implant with a second removable cartridge containing the second implant.

In Example 63, one or more of steps 2-7 of any one or any combination of Examples 59-62 may be performed by a user engaging one or more actuators on the delivery device.

In Example 64, the one or more of the separate removable cartridges of Example 62 may include a spooling mechanism or assembly.

In Example 65, the spooling mechanism or assembly of Example 64 may be configured to engage or couple with distal anchor portion and/or middle portion.

In Example 66, the handle assembly of any one or any combination of Example 62-65 may include a spooling mechanism or assembly.

In Example 67, the handle assembly of Example 66 may be configured to engage or couple with the distal anchor portion, middle portion, and/or needle.

In Example 68, the removable cartridge of any one or any combination of Examples 62-67 may include, be attached to, or be coupled with the needle.

In Example 69, the removable cartridge of Example 68 may be inserted into a side portion of the handle assembly.

In Example 70, the removable cartridge of Example 68 may be inserted into a rear portion of the handle assembly.

In Example 71, the delivery device of any one or any combination of Examples 59-61 may include a handle assembly and two or more implants.

In Example 72, the delivery device of Example 71 may not include a removable cartridge, such that cartridge exchange between successive implant deployments is not necessary.

In Example 73, the delivery device of one or both of Examples 71 and 72 may include at least one spooling mechanism or assembly configured to simultaneously store and serially deploy multiple implants.

In Example 74, implant deployment utilizing the delivery device of Example 73 may involve a user engaging one or more actuators on the delivery device one or more times.

In Example 75, the delivery device of any one or any combination of Examples 59-61 may include a handle assembly and two or more implants.

In Example 76, each of the two or more implants of Example 75 may include an enlarged feature at a proximal end thereof.

In Example 77, the delivery device of Example 75 and/or 76 may include a magazine component or assembly configured to store at least a portion of each implant for serial deployment.

In Example 78, deploying each implant of Example 77 may involve actuating the delivery device to move a push member distally through a shaft assembly of the device, the push member engaging an implant to move it distally.

In Example 79, the magazine assembly of Example 77 and/or 78 may be attached or coupled with a needle assembly.

In Example 80, the delivery device of any one or any combination of Examples 59-79 may include an indexing assembly or mechanism configured to automatically index each implant or portion thereof for serial deployment during a procedure.

In Example 81, a delivery device of, or utilized pursuant to, any one or any combination of Examples 1-80 may be automated in whole or in part.

In Example 82, the delivery device of Example 81 may include one or more electromechanical components.

In Example 83, the delivery device of Example 81 and/or 82 may further include a battery.

In Example 84, the delivery device of any one or any combination of Examples 81-83 may further include or be coupled with one or more user displays or interfaces.

In Example 85, the delivery device of any one or any combination of Examples 81-84 may further include one or more force sensors configured to measure suture tension and/or tissue compression.

In Example 86, the delivery device of any one or any combination of Examples 81-85 may further include means for detecting and/or visualizing deployed implants.

In Example 87, the delivery device of Example 86 may further include means for displaying the detected and/or visualized implants.

In Example 88, the delivery device of any one or any combination of Examples 81-87 may further include a means for determining a position of a distal end of an elongate shaft assembly.

In Example 89, the delivery device of Example 88 may further include means for determining a distance of the distal end of the elongate shaft assembly from a treatment site within a prostatic urethra.

In Example 90, the delivery device of any one or any combination of Examples 81-89 may further include control circuitry configured to implement one or more automated actions of the device.

In Example 91, the delivery device of any one or any combination of Examples 81-90 may further include a motorized cam system configured to operate CT/suture indexing and/or UE indexing.

In Example 92, the delivery device of any one or any combination of Examples 81-91 may further include a position sensor configured to detect CT and/or UE locations after deployment.

In Example 93, the delivery device of any one or any combination of Examples 81-92 may further include a needle speed control mechanism configured to vary a distal advancement speed of the needle.

In Example 94, one or more components of or coupled with the device of any one or any combination of Examples 91-93 may be disposable.

In Example 95, one or more components of or coupled with the device of any one or any combination of Examples 91-94 may be motorized.

In Example 96, a delivery device configured to deploy multiple implant assemblies to a prostate gland of a patient without removing an elongate delivery shaft of the delivery device from the patient includes an elongate delivery shaft attached to a handle assembly. The handle assembly may include a first spool member configured to couple with multiple implant assemblies simultaneously, each implant assembly comprising a distal anchor component and a suture attached thereto (“CT/suture”). The distal anchor component may be configured to anchor to a prostatic capsule of the prostate gland, and the suture may be configured to be placed within the prostate gland. The handle assembly may further include a second spool member configured to couple with a delivery needle configured to pierce the prostate gland. The handle assembly may further include one or more actuators configured to deploy each implant assembly via a ratcheting mechanism.

In Example 97, at least one of the one or more actuators of Example 96 may be directly or operatively coupled to the first spool member, the second spool member, or both.

In Example 98, the one or more actuators of Examples 96 and/or 97 may include a first actuator and a second actuator

In Example 99, actuation of the first actuator of Example 98 may drive deployment of each implant assembly.

In Example 100, the handle assembly of any one or any combination of Examples 96-99 may contain multiple urethral endpieces attachable to the implant assemblies, where actuation of the second actuator may index the urethral endpieces in the elongate delivery shaft.

In Example 101, the first actuator of Example 99 comprises a trigger.

In Example 102, the second actuator of Example 100 comprises a lever member.

In Example 103, the first spool member and the second spool member of any one or any combination of Examples 96-102 may rotate at different speeds in response to actuation of at least one of the one or more actuators.

In Example 104, the second spool member of any one or any combination of Examples 96-103 may be further configured to unwind and wind the delivery needle in response to actuation of at least one of the one or more actuators.

In Example 105, the second spool member of any one or any combination of Examples 96-104 may be coupled with a gear member.

In Example 106, the gear member of Example 105 may be attached to a torsion spring.

In Example 107, the ratcheting mechanism of any one or any combination of Examples 96-106 may include an assembly comprising a ratchet plate defining a suture deployment track.

In Example 108, the suture deployment track of Example 107 may be configured to receive and accommodate stepwise movement of a movable component.

In Example 109, the movable component of Example 108 may include a pin member configured to slide within the suture deployment track.

In Example 110, the assembly of Example 108 may further include a return plate defining a suture return track, the suture return track also configured to receive and accommodate stepwise movement of the movable component.

In Example 111, repeated actuation of the one or more actuators of Example 108 may cause the stepwise movement of the movable component.

In Example 112, the first spool member of any one or any combination of Examples 96-111 may be configured to move relative to the second spool member.

In Example 113, the first spool member of Example 112 may define a plurality of circumferential tracks, each circumferential track configured to accommodate one implant assembly.

In Example 114, movement of the first spool member relative to the second spool member of Example 113 may cause the implant assemblies to unwind from the first spool member and feed into the delivery needle via an opening defined by the second spool member.

In Example 115, the urethral endpieces of Example 100 are aligned end-to-end within the elongate delivery shaft.

In Example 116, a method of deploying an implant using the delivery device of any one or any combination of Examples 96-115 may involve actuating at least one of the one or more actuators three or more times.

In Example 117, implementing the method of Example 116 causes compression of enlarged tissue into which the implant is deployed.

In Example 118, implementing the method of Example 116 causes compression of prostate tissue.

In Example 119, implementing the method of any one or any combination of Examples 116-118 treats benign prostatic hyperplasia.

In various examples and embodiments described herein, separate components may be considered or referred to as part of the same delivery device. For example, examples may feature a delivery device that includes a handle assembly and a removable cartridge. Such examples may also be considered or referred to as systems, such as, for example, a system that includes a handle assembly and a removable cartridge configured to couple therewith. The implant assemblies and components described herein, e.g., CT/suture assemblies and UEs, may similarly be considered components of a single delivery device or components of a system that includes a delivery device.

### CLOSING NOTES

Certain terms are used throughout this patent document to refer to particular features or components. As one skilled in the art appreciates, different people may refer to the same feature or component by different names. This patent document does not intend to distinguish between components or features that differ in name but not in function.

For the following defined terms, certain definitions shall be applied unless a different definition is given elsewhere in this patent document. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” generally refers to a range of numbers that one of skill in the art would consider functionally equivalent to the recited value (e.g., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.). The terms “patient” and “subject” are intended to include mammals, such as for human or veterinary applications. The terms “distal” and “proximal” are used to refer to a position or direction relative to the treating clinician. “Distal” and “distally” refer to a position that is distant from, or in a direction away from, the treating clinician. “Proximal” and “proximally” refer to a position that is near, or in a direction toward, the treating clinician.

The scope of the invention should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device, kit or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A delivery device configured to deploy multiple implant assemblies to a prostate gland of a patient without removing an elongate delivery shaft of the delivery device from the patient, comprising:
an elongate delivery shaft attached to a handle assembly, the handle assembly comprising:
a first spool member configured to couple with multiple implant assemblies simultaneously, each implant assembly comprising a distal anchor component and a suture attached thereto (“CT/suture”), the distal anchor component configured to anchor to a prostatic capsule of the prostate gland, and the suture configured to be placed within the prostate gland;
a second spool member configured to couple with a delivery needle configured to pierce the prostate gland; and
one or more actuators configured to cause deployment of the implant assemblies in serial fashion.

2. The delivery device of claim 1, wherein the one or more actuators are manually engageable.

3. The delivery device of claim 1, wherein at least one of the one or more actuators comprises a gearing portion inside the handle assembly.

4. The delivery device of claim 1, wherein at least one of the one or more actuators is directly or operatively coupled to the first spool member, the second spool member, or both.

5. The delivery device of claim 1, wherein the delivery device further includes multiple urethral endpieces attachable to the implant assemblies, and wherein the one or more actuators are further configured to cause attachment of each of the implant assemblies to one of the urethral endpieces.

6. The delivery device of claim 5, wherein the one or more actuators comprises a first actuator and a second actuator.

7. The delivery device of claim 6, wherein actuation of the first actuator drives deployment of each implant assembly.

8. The delivery device of claim 7, wherein actuation of the second actuator indexes the urethral endpieces in the elongate delivery shaft.

9. The delivery device of claim 7, wherein the first actuator comprises a trigger.

10. The delivery device of claim 8, wherein the second actuator comprises a lever member.

11. The delivery device of claim 1, wherein the first spool member and the second spool member rotate at different speeds in response to actuation of at least one of the one or more actuators.

12. The delivery device of claim 1, wherein the second spool member is further configured to unwind and wind the delivery needle in response to actuation of at least one of the one or more actuators.

13. The delivery device of claim 1, wherein the one or more actuators are configured to cause deployment of the implant assemblies in serial fashion via a ratcheting mechanism.

14. The delivery device of claim 13, wherein the ratcheting mechanism comprises an assembly comprising a ratchet plate defining a suture deployment track configured to receive and accommodate stepwise movement of a movable component.

15. The delivery device of claim 1, wherein the first spool member is configured to move relative to the second spool member.

16. The delivery device of claim 1, wherein the first spool member defines a plurality of circumferential tracks, each circumferential track configured to accommodate one implant assembly.

17. The delivery device of claim 16, wherein movement of the first spool member relative to the second spool member causes the implant assemblies to unwind from the first spool member and feed into the delivery needle via an opening defined by the second spool member.

18. The delivery device of claim 5, wherein the urethral endpieces are aligned end-to-end within the elongate delivery shaft.

19. A method for compressing prostate tissue, the method comprising:
advancing an elongate shaft assembly of a delivery device through a urethra until a distal end of the elongate shaft assembly is positioned adjacent to the prostate tissue, wherein the delivery device further comprises a handle assembly attached to a proximal end of the elongate shaft assembly, the handle assembly comprising:
a spooling assembly comprising a first spool member and a second spool member, the first spool member configured to couple with multiple prostatic implant assemblies simultaneously, and the second spool member configured to couple with a hollow delivery needle configured to receive the prostatic implant assemblies and pierce the prostate tissue; and
one or more manually engageable actuators configured to cause deployment of the prostatic implant assemblies into the prostate tissue in serial fashion; and
deploying the prostatic implant assemblies into the prostate tissue without removing the elongate shaft assembly from the urethra.

20. The method of claim 19, wherein deploying the prostatic implant assemblies into the prostate tissue comprises actuating at least one of the one or more manually engageable actuators two or more times.
